(19) United States
(12) Patent Application Publication
(10) Pub. No.: US 2004/0171659 A1
(43) Pub. Date: Sep. 2, 2004

(54) Methods for treating diabetes

(76) Inventors: Satyanarayana Medicherla, Cupertino, CA (US); Andrew A. Protter, Palo Alto, CA (US); George F. Schreiner, Los Altos, CA (US)

Correspondence Address: MORRISON & FOERSTER LLP, 3811 VALLEY CENTRE DRIVE, SUITE 500, SAN DIEGO, CA, 92130-2332, US

(21) Appl. No.: 10/728,665

(22) Filed: Dec. 5, 2003

Related U.S. Application Data
(60) Provisional application No. 60/431,241, filed on Dec. 6, 2002.

(51) Int. Cl. A61K 31/425

(52) U.S. Cl. 514/369

(57) ABSTRACT

The invention is directed to methods of treating diabetes by administering p38 mitogen activated protein kinase inhibitors. The invention is also directed to methods of decreasing blood glucose level in diabetes patients by administering p38 mitogen activated protein kinase inhibitors.

### CROSS-REFERENCE TO RELATED APPLICATIONS

This application claims the benefit of priority of U.S. Provisional Patent Application No. 60/431,241 filed Dec. 6, 2002, the contents of which are hereby incorporated by reference.

### BACKGROUND OF THE INVENTION

1. Technical Field

The invention relates to methods of treating diabetes by administering p38 mitogen activated protein kinase (p38 MAPK) inhibitors.

2. Background Art

Diabetes is caused by occurrence of abnormal metabolisms of glucose, protein and lipid due to a deficiency or insufficiency of the actions of insulin. Typical signs of diabetes include an abnormal increase in the serum glucose level over the normal range of the glucose level and an excretion of glucose in the urine.

Type 1 diabetes is an autoimmune disease and becomes clinically evident when the majority of endocrine beta cells have been destroyed (Yoshida, K. and Kikutani, H., Reviews in Immunogenetics 2:140-146 (2000)). Because the development of Type 1 diabetes in certain people can now be predicted, investigations have begun to explore the use of intervention therapy to halt or even prevent beta cell destruction in such individuals (Ryu, S. et al., J. Clin. Invest. 108:63-72 (2001); Mahon, J. L. et al., Ann. N.Y. Acad. Sci. 696:351-363 (1993); Shapiro, A. M. et al., Diabetologia 45:224-230 (2002); Debussche, X. et al., Diabete & Metabolisme (Paris) 20:282-290 (1994); Keymeulen, B. and G. Somer, Acta Clinica Belgica 48:86-95 (1993)). In Type 1 diabetes, a honeymoon refractory period is generally observed where insulin level appears to be normalized. This is a transient period which can last for weeks to months, followed by complete onset of diabetes. With the currently available therapy, 60-80% of patients will reach the honeymoon period (Keymeulen, B. and Somer, G., Acta Clinica Belgica 48:86-95 (1993)). Now, the challenge is to indefinitely prolong the duration of the honeymoon period of Type 1 diabetes (Hosker, J.P. and Turner, R. C., Lancet 18:633-635 (1982); Heinze, E. and Thon, A., Pediatrician 12:208-212 (1985); Crump, W. J., J. Family Practice 25:78-82 (1987); Palmer, J.P. and McCulloch, D. K., Diabetes 40:943-947 (1990)). Numerous attempts have been made to prolong the honeymoon period by immunotherapy to interrupt the ongoing self-destruction of the insulin-producing beta cells (Shapiro, A. M. et al., Diabetologia 45:224-230 (2002); Herold, K. C. et al., New Engl. J. Med. 346:1682-1698 (2002)).

It is very clear by now that T lymphocytes play a key role in the destruction of insulin producing beta cells. Evidence from animal experiments suggests that CD⁺4 (helper) and CD⁺8 (cytotoxic) T cells are required for the development of insulitis (Thomas, H. E. and Kay, T. W. H., Diabetes/Metabolism Res. Rev. 16:251-261 (2000); Hancock, W. W. et al., Am. J. Pathol. 147:1194-1199 (1995); Salomon, B. et al., Immunity 12:431-437 (2000)). The final destruction of beta cells is probably the result of many factors, being dependent on multiple cell types (macrophages, CD⁺4 and CD⁺8 positive T lymphocytes) and multiple mechanisms (free-radical damage, interleukin-1, CD⁺8 T cell-mediated toxicity, activated p38) (Thomas, H. E. and Kay, T. W. H., Diabetes/Metabolism Res. Rev. 16:251-261 (2000); Hancock, W. W. et al., Am. J. Patho. 147:1194-1199 (1995); Zhang, J. et al, International Immunology 13:377-384 (2001)). The non-obese diabetic (NOD) mouse spontaneously develops Type 1 diabetes and has many immunological and pathological similarities to human Type 1 diabetes (Yoshida, K. and Kikutani, H., Rev. Immunogenetics 2:140-146 (2000)). Therefore, the NOD mouse has served as one of the primary models for Type 1 diabetes and a model to test new approaches for immunotherapy.

A wide array of immunosuppressive agents have been shown capable of preventing the onset of Type 1 diabetes in NOD mice and in humans (Shapiro, A. M. et al., Diabetologia 45:224-230 (2002); Casteels, K. et al., Transplantation 65:1225-1232 (1998); Tabatabaie, T. et al., Biochem. Biophys. Res. Comm. 273:699-704 (2000); Mori, Y. et al., Diabetologia 29:244-247 (1986)). Most of these agents have been used prophylactically before the appearance of overt diabetes and even often before insulitis (Shapiro, A. M. et al., Diabetologia 45:224-230 (2002); Mori, Y. et al., Diabetologia 29:244-247 (1986)). Unfortunately, no drug has been convincingly shown so far to be as efficient as the immunosuppressive agent cyclosporin with less toxicity (Mahon, J. L. et al., Ann. N.Y. Acad. Sci. 696:351-363 (1993); Tabatabaie, T. et al., Biochem Biophys. Res. Comm. 273:699-704 (2000)).

Protein kinases are involved in various cellular responses to extracellular signals. p38 mitogen activated protein (MAP) kinase (also called p38 kinase, p38 MAPK, or “High Osmolarity Glycerol response kinase” (HOG)) is a member of a family of signaling molecules known as the Mitogen-Activated Protein kinase (MAP kinase or MAPK) family. Other members of the MAP kinase family include the classical MAPKs termed Extracellular signal Regulated Kinases (ERK), which are activated by a variety of mitogenic stimuli as well as differentiation signals, and Stress-Activated Protein Kinases (SAPK) (also called Jun N-terminal Kinases (JNK)). SAPKs are activated by stresses but not mitogens, like the p38 MAP kinase.

p38 MAP kinase is activated by a variety of cellular stressors, including ultraviolet radiation, osmotic shock, and inflammatory cytokines, such as interleukin-1 (IL-1) and tumor necrosis factor-α(TNF-α). Once activated, p38 MAP kinase mediates the induction of mRNA synthesis for a variety of inflammatory mediators, including IL-1 P, TNF-α, IL-6, and cyclo-oxygenase-2 (COX-2).

Inhibition of p38 MAP kinase leads to a blockade on the production of both IL-1 and TNF. IL-1 and TNF stimulate the production of other proinflammatory cytokines such as IL-6 and IL-8 and have been implicated in acute and chronic inflammatory diseases and in postmenopausal osteoporosis (Kimble, R. B. et al., Endocrinol. 136:3054-3061 (1995)). Based upon this finding it is believed that p38 MAP kinase, along with other MAPKs, have a role in mediating cellular response to inflammatory stimuli, such as leukocyte accumulation, macrophage/monocyte activation, tissue resorption, fever, acute phase responses and neutrophilia. In addition, MAPKs, such as p38 MAP kinase, have been implicated in cancer, thrombin-induced platelet aggregation, immunodeficiency disorders, autoimmune diseases, cell death, allergies, osteoporosis and neurodegenerative disorders. Other diseases associated with IL-1, IL-6, IL-8 or TNF overproduction are set forth in WO 96/21654.

After the discovery of p38 MAPK in 1994, considerable interest has been given to the MAPK super-family owing to the activation of leukocytes, which play a central role during inflammatory responses and in autoimmune diseases (Badger, A.M., J. Pharmacol. Exp. Ther. 279:1453-1461 (1996); Rogers, D. F. and Giembycz, M. A., Drug Discov. Today 3:532-535 (1998)). Treatment with p38 MAP kinase inhibitors attenuates both p38 activation and disease severity (Jackson, J. R., J. Pharmacol. Exp. Ther. 284:687-692 (1998)). Structurally diverse p38 MAP kinase inhibitors have been tested extensively in several inflammatory disease models (Wang, Z., Structure 6:1117-1128 (1998); Herlaar, E. and Brown, Z., Molec. Med. Today 5:439-447 (1999)). Out of the four members of the p38 MAP kinase family, the isoform p38α is best studied (Herlaar, E. and Brown, Z., Molec. Med. Today 5:439-447 (1999)). When activated as an integral part of leukocyte activation in acute and chronic inflammatory states, p38α MAP kinase plays a key role in the disease process (Hale, K. K. et al., J. Immunol. 162:4246-4252 (1999); Lee, J. C., et al., Immunopharmacology 47:185-201 (2000)). In fact, it is the major p38 isoform expressed by human monocytes, activated macrophages, neutrophils and CD⁺4 T cells, suggesting that development of inhibitors that have p38 activity, preferably p38α activity, might be of significant therapeutic benefit (Herlaar, E. and Brown, Z., Molec. Med. Today 5:439-447 (1999)).

### SUMMARY OF THE INVENTION

The invention is directed to a method of treating diabetes in a patient, the method comprising administering to the patient a pharmaceutically effective amount of a p38 mitogen activated protein (MAP) kinase inhibitor sufficient to treat diabetes in the patient.

The invention is also directed to a method of decreasing blood glucose level in a diabetes patient, the method comprising administering to the patient a pharmaceutically effective amount of a p38 mitogen activated protein (MAP) kinase inhibitor sufficient to decrease blood glucose level in the patient.

The invention is also directed to a method of treating insulitis in a patient, the method comprising administering to the subject a pharmaceutically effective amount of a p38 mitogen activated protein (MAP) kinase inhibitor sufficient to treat insulitis in the patient.

### BRIEF DESCRIPTION OF THE DRAWINGS

FIGS. 1A-1D. Preventive effects of p38 MAP kinase inhibitor on development of diabetes in NOD mice. Pre-diabetic NOD mice treated with p38 MAP kinase inhibitor (“p38 inhibitor”) for 10 weeks had higher body weights (*p<0.05 vs. vehicle group) (FIG. 1A); lower blood glucose levels (*p<0.05 and **p<0.01 vs. vehicle group) (FIG. 1B); and higher insulin levels (*p<0.05 vs. vehicle group) (FIG. 1C) when compared to the vehicle treated group. Values are reported as the mean±SEM (n=20). There was a statistically significant (*p<0.01 vs. vehicle group) and dose-dependent delay in the onset of diabetes as defined by blood glucose levels greater 120 mg/dl (FIG. 1D). Open circles are vehicle group. Open triangles are p38 MAP kinase inhibitor at low dose and hatched circles are p38 MAP kinase inhibitor at high dose groups. By 18 weeks 60% of the mice fed standard chow (vehicle) had developed diabetes, while in the low dose and high dose p38 MAP kinase inhibitor treated groups only 30% and 10% of mice developed diabetes.

FIGS. 2A-2D. Pancreata of p38 MAP kinase inhibitor and vehicle treated mice were histologically (H&E) examined after 10 weeks of treatment. The pancreata of NOD mice from vehicle group showed destruction of islets of Langerhans with a severe lymphocytic infiltration (FIG. 2A). In contrast, the pancreata of mice treated with p38 MAP kinase inhibitor both at low and high doses showed only minor lymphocyte infiltration (FIGS. 2B, 2C). Quantitative histological assessment showed that p38 MAP kinase inhibitor treatment at both doses significantly (*p<0.05 vs. vehicle group) suppressed insulitis scores (FIG. 2D). Open circles are vehicle group. Open triangles are p38 MAP kinase inhibitor at low dose and hatched circles are p38 MAP kinase inhibitor at high dose groups. Values are reported as the mean±SEM (n=20).

FIGS. 3A-3D. Inmmunohistochemical staining for CD⁺4 (FIGS. 3A, 3C) and CD⁺8 (FIGS. 3B, 3D) T cells in the islets of vehicle (FIGS. 3A, 3B) and p38 MAP kinase inhibitor (FIGS. 3C, 3D) treated NOD mice. In mice treated with and without p38 MAP kinase inhibitor for 10 weeks, 90% of the infiltrating lymphocytes were shown by immunohistochemistry to be CD⁺5 T cells. 80% of the infiltrating T cells were CD⁺4 and 20% were CD⁺8. Treatment with p38 MAP kinase inhibitor at high dose remarkably suppressed CD⁺4 (FIG. 3C) and CD⁺8 (FIG. 3D) T cells infiltration into the beta cells without affecting their ratio.

FIGS. 4A-4C. p38 expression in the T cells infiltrated into the beta cells of vehicle (FIG. 4A) and p38 MAP kinase inhibitor (FIG. 4B) treated NOD mice. After 10 weeks of treatment, enhanced p38 MAP kinase expression was observed (see arrows) both in cytoplasm and nucleus of the T cells infiltrated into the beta cell mass of the vehicle treated group. In contrast, p38 MAP kinase inhibitor significantly reduced p38 MAP kinase expression in the T cells. Summarized results on p38 expression are shown as grades (FIG. 4C). Treatment with p38 MAP kinase inhibitor significantly decreased the p38 expression in the T cells (*p<0.001 vs. vehicle group). Open bars are vehicle and hatched bars are p38 MAP kinase inhibitor at high dose group. Values are reported as the mean±SEM (n=14).

FIGS. 5A-5C. Therapeutic effects of p38 MAP kinase inhibitor on blood glucose levels in mildly hyperglycemic NOD mice. Mildly hyperglycemic NOD mice treated with p38 MAP kinase inhibitor for 17 days had decrease in weight loss (*p<0.05 vs. vehicle group) (FIG. 5A); and higher insulin levels (FIG. 5B) when compared to the vehicle treated group. In vehicle-treated NOD mice, (severe) hyperglycemia developed significantly by day-17 when compared to its baseline value. Whereas, p38 MAP kinase inhibitor dose-dependently prevented the development of hyperglycemia and the mice are mildly hyperglycemic by day-17 (FIG. 5C). Values are reported as the mean±SEM (n=7). *p<0.01 vs. baseline value. Open circles are vehicle, open triangles are p38 MAP kinase inhibitor at low dose, and hatched circles are p38 MAP kinase inhibitor at high dose groups.

FIGS. 6A-6C. Therapeutic effects of p38 MAP kinase inhibitor on blood glucose levels in mildly hyperglycemic NOD mice. Mildly hyperglycemic NOD mice treated with high dose of p38 MAP kinase inhibitor for 17 days had lower fasting blood glucose levels (*p<0.05 vs. vehicle group) (FIG. 6A) when compared to the vehicle treated group. Oral glucose tolerance was evaluated on day 17 following an overnight fast. Blood glucose was measured immediately prior to and 30, 60, and 120 minutes following an oral glucose challenge (2 g/kg). Open bars/circles are vehicle and hatched bars/circles are p38 MAP kinase inhibitor at high dose group. Values are reported as the mean±SEM (n=6). p38 MAP kinase inhibitor for 17 days had improved glucose tolerance (*p<0.05 vs. vehicle group) when compared to the vehicle group (FIG. 6B). p38 MAP kinase inhibitor at high dose showed highly significant improvement in glucose tolerance at 30 minutes of the test (*p<0.001 vs. vehicle group) (FIG. 6C).

FIG. 7 is a bar graph showing the percent incidence of diabetes in the test NOD animals treated with a p38 MAPK inhibitor. Mice administered the p38 MAPK inhibitor suffered a lower percentage of diabetes than those mice that received vehicle alone.

FIG. 8 plots blood glucose levels in mg/dl in test and control NOD mice at 13 and 18 weeks. The data show that that mice receiving the p38 MAPK inhibitor had lower blood glucose levels than those receiving vehicle alone.

FIGS. 9A-9D show immunohistochemical analysis of pancreata from test NOD mice receiving a p38 inhibitor and control mice receiving vehicle alone at 13 and 18 weeks.

FIG. 10 shows a bar graph indicating that administration of a p38 MAPK inhibitor lowers HSP 60 expression in NOD mice as 13 and 18 weeks.

### MODES OF CARRYING OUT THE INVENTION

In the present study, the ability of a p38 MAP kinase inhibitor to suppress T cell infiltration in the pancreatic beta cell mass of the NOD mice was examined. It has been discovered as provided in the present invention that the p38 MAP kinase inhibitor prevents development of diabetes and alleviates hyperglycemia in NOD mice.

The invention is directed to a method of treating diabetes in a patient, the method comprising administering to the patient a pharmaceutically effective amount of a p38 mitogen activated protein (MAP) kinase inhibitor sufficient to treat diabetes in the patient.

In another embodiment, the invention is directed to a method of decreasing blood glucose level in a diabetes patient, the method comprising administering to the patient a pharmaceutically effective amount of a p38 mitogen activated protein (MAP) kinase inhibitor sufficient to decrease blood glucose level in the patient.

In a further embodiment, the invention is directed to a method of treating insulitis in a patient, the method comprising administering to the subject a pharmaceutically effective amount of a p38 mitogen activated protein (MAP) kinase inhibitor sufficient to treat insulitis in the patient.

“Diabetes” or diabetes mellitus is a metabolic disease that is defined by the presence of chronically elevated levels of blood glucose. Diabetes is caused by abnormal metabolism of glucose, protein and lipid, due to a deficiency or insufficiency of the actions of insulin. Typical signs of diabetes include an abnormal increase in the serum glucose level over the normal range of the glucose level and an excretion of glucose in the urine. Classic symptoms of diabetes mellitus in adults include polyuria, polydipsia, ketonuria, rapid weight loss, other acute manifestations of hyperglycemia, and elevated levels of plasma glucose.

More specifically, diabetes is a disease wherein the blood glucose level, which should usually be controlled at about 100 to about 200 mg/dl, is abnormally raised. Diagnostic criteria for diabetes include, but are not limited to: symptoms of diabetes plus casual plasma glucose of about or greater than 200 mg/dL; fasting plasma glucose of about or greater than 126 mg/dL, confirmed by repeat testing on a different day; or plasma glucose of about or greater than 200 mg/dL at 2 hours after a 75-g oral glucose challenge, confirmed by repeat testing on a different day. Fasting is defined as no caloric intake for at least 8 hours. See, Harris, M. I., “Definition and Classification of Diabetes Mellitus and the New Criteria for Diagnosis,” pp. 326-334, Table 32-3, in: DIABETES MELLITUS a Fundamental and Clinical Text, LeRoith D. et al. Eds., 2^{nd} ed. (2000). Skill artisans can readily diagnose diabetes and its symptoms based on the knowledge in the art.

Insulin is one of the hormones in the pancreas and promotes permeability of glucose through the cell membranes in liver, muscles and adipose tissues, and increase the uptake of glucose by the cells. Insulin also promotes metabolism of glucose in the glycolysis step and oxidation step of glucose in the muscles and increases the activity of the enzyme system for synthesizing glycogen from glucose. By exhibiting these biological functions, insulin acts to keep the serum glucose level at normal levels.

An expert committee of the American Diabetes Association proposed the following diabetes classification scheme, which provides guidelines for classification of diabetes for the present invention. Type 1 diabetes mellitus is caused by beta cell destruction that leads to loss of insulin secretion and complete insulin deficiency. Type 1 occurs in about 5% to 10% of diabetes cases. Type 2 diabetes mellitus is caused by a combination of genetic and nongenetic factors that result in insulin resistance and insulin deficiency. Nongenetic factors include increasing age, high caloric intake, overweight, central adiposity, sedentary lifestyle, and low birth weight. Type 2 occurs in about 90% to 95% of diabetes cases. Other specific types of diabetes mellitus is a heterogeneous etiologic group that includes those cases of diabetes in which the causes are established or at least partially known. The causes include known genetic defects affecting beta cell function or insulin action, diseases of the exocrine pancreas, endocrinopathies, drug- or chemical-induced pancreatic changes, and diseases and conditions in which the incidence of diabetes is substantially elevated but a precise etiology has not been established. Other specific types occur in about 1% to 2% of diabetes cases. Gestational diabetes mellitus is caused by insulin resistance and relative insulin deficiency associated with pregnancy and occurs in about 3% to 5% of all pregnancies. See, Harris, M. I., “Definition and Classification of Diabetes Mellitus and the New Crieria for Diagnosis,” pp. 326-334, Table 32-1, in: DIABETES MELLITUS a Fundamental and Clinical Text, LeRoith D. et al. Eds., 2 ed. (2000).

Type 1 diabetes (also called insulin-dependent diabetes (IDDM), juvenile diabetes, brittle diabetes, or sugar diabetes) is accompanied by reduction of insulin producing cells, and Type 2 (also called non-insulin-dependent diabetes (NIDDM)) is caused by insulin sensitivity reduction or insulin secretion reduction.

Type 1 is the most common form among children and adolescents, in which the disease is usually characterized by abrupt onset of severe symptoms, dependence on exogenous insulin to sustain life, and proneness to ketosis even in the basal state, all caused by absolute insulin deficiency (insulinopenia). Onset in adults can occur. In adults, the rate of beta cell destruction appears to be slower than in children, and residual beta cell function sufficient to prevent ketoacidosis may be present for many years.

There are two forms of Type 1 diabetes. Idiopathic Type 1 refers to rare forms of the disease with no known cause. Immune-mediated Type 1 diabetes is an autoimmune disorder in which the immune system destroys, or attempts to destroy, the beta cells in the pancreas that produce insulin in response to elevated plasma glucose levels.

“Insulitis” refers to infiltration of lymphocytes into pancreatic islets of Langerhans and destruction of beta cells. Insulitis is the most prominent histopathologic lesions in NOD mice (Makino, S. et al., Exp. Animal 29:1-13 (1980)). The first signs of pancreatic perivasculitis are seen at an age of 15 days (Sugihara, T. et al., Histol. Histopathol. 4:397-404 (1989), Miyazaki, A. et al., Clin. Exp. Immuno. 60:622-630 (1985)). Islet infiltration is noticeable at 4 weeks and at the same time degeneration in islet beta cells may be observed by light and electron microscopy (mice (Makino, S. et al., Exp. Animal 29:1-13 (1980)). The lymphocyte infiltrations are predominantly T-lymphocytes (Thy-1.2+), most often CD5+ (Ly-1+) and some CD8+ (Lyt-2+, -3+). B-lymphocytes are also found in the cellular infiltration, and anti-islet-cell antibodies (ICSA) are present in the plasma in the prediabetic stage. Lymphocyte infiltration is also observed in other organs such as salivary glands, thyroid glands, adrenal glands, testes, and ovaries. Upon complete onset of Type 1 diabetes, all of the pancreatic beta cells have been destroyed.

Symptoms of Type 1 diabetes include, but are not limited to, high levels of sugar in the blood when tested, high levels of sugar in the urine when tested, unusual thirst, frequent urination, extreme hunger but loss of body weight, blurred vision, nausea and vomiting, extreme weakness and tiredness, and irritability and mood changes.

Complications associated with Type 1 include, but are not limited to, hypoglycemia (blood sugar drops too low, called insulin reaction), hyperglycemia (blood sugar is too high, indicating diabetes is not well controlled), and ketoacidosis (diabetic coma or loss of consciousness due to untreated or under-treated diabetes).

In islet cells, proinsulin is cleaved into an insulin molecule and a C-peptide molecule, and insulin is released into the circulation at concentrations equimolar to that of C-peptide. C-peptide can be used as a marker for insulin secretion and assays using the plasma level of C-peptide as a index of beta cell function. This marker can be predictive of diabetic patients.

Subjects at high risk for Type 1 diabetes can now be identified (Ryu, S. et al., J. Clin. Invest. 108:63-72 (2001); Mahon, J. L. et al., Ann. N.Y. Acad. Sci. 696:351-363 (1993); Shapiro, A. M. et al., Diabetologia 45:224-230 (2002)). A major goal of the present invention is to reduce the incidence of diabetes by disease-specific nontoxic agents. During development of Type 1 diabetes, as beta cell function decreases and hyperglycemia prevails, very low doses of insulin can suffice to maintain normal blood glucose. A metabolic or transient remission, called the “honeymoon period,” wherein about 10% to about 20% of beta cells remain, can occur prior to complete onset of Type 1 diabetes (characterized by complete destruction of beta cells). An object of the present invention is to prolong the honeymoon period or to delay the complete onset of Type 1 diabetes, preferably indefinitely, through the use of p38 MAP kinase inhibitors.

Type 2 diabetes is characterized by insulin resistance, i.e., a failure of the normal metabolic response of peripheral tissues to the action of insulin. Insulin resistance refers to a condition wherein the insulin level required to exhibit insulin activity at the same level as a healthy person is much higher than that of the healthy person. It is a condition wherein the activity of insulin or sensitivity for insulin is reduced. In clinical terms, insulin resistance is when normal or elevated blood glucose levels persist in the presence of normal or elevated levels of insulin.

The hyperglycemia associated with Type 2 diabetes can be reversed or ameliorated by diet or weight loss sufficient to restore the sensitivity of the peripheral tissues to insulin. Type 2 diabetes is often characterized by hyperglycemia in the presence of higher than normal levels of plasma insulin. Progression of Type 2 diabetes includes increasing concentrations of blood glucose, coupled with a relative decrease in the rate of glucose-induced insulin secretion. Thus, for example, in late-stage Type 2 diabetes, insulin deficiency can occur. Unlike the pancreatic beta cells in Type 1 diabetics, the beta cells of Type 2 diabetics retain the ability to synthesize and secrete insulin.

The target organs for insulin activity include liver, muscle (skeletal muscle), and adipose tissue. Insulin exhibits a gluconeogenesis suppression activity, a glucose release suppression activity, etc. in the liver. Insulin exhibits glucose-uptake promoting activity in the muscle (skeletal muscle) and adipose tissues.

It is considered that hyperglycemia per se inhibits the insulin secretion, and exacerbates the insulin resistance. Thus, there is a “glucose-toxicity” theory that hyperglycemia per se is an important cause for diabetes which exacerbates the metabolism abnormality (edited by Takashi KADOWAKI, Molecular Medicine for Diabetes, published by Yodo Co. (1992)).

In addition, Type 2 diabetes mellitus is often accompanied by hyperlipemia, and an unusual high level of cholesterol or triglyceride (i.e., more than about 220 mg/dl of total cholesterol, or more than about 150 mg/ml of triglyceride) is a risk factor to cause arteriosclerosis including myocardial infarction, or acute pancreatitis. Hyperlipemia is caused by a genetic diathesis such as a familial hypercholesterolemia, obesity, or hyperphagia or epicurism. Moreover, the hyperlipemia is classified into a chylomicronemia (type I), a hyperchloresterolemia (type IIa hyperlipemia), a hypertriglyceridema (type IV), or a combination thereof (type IIb or type III), etc., in terms of the symptoms thereof. However, an essential drug therapy for hyperlipemia has not been established yet, and diabetic complications caused by abnormal continuous blood lipid level, such as arteriosclerosis, ischemic heart diseases, are one of the main causes for death in the developed countries.

Type 2 diabetes mellitus is often accompanied by obesity. Obesity is considered to relate to hypertension, or to the vascular disorders of the brain and the heart, in the epidemiology research, and obesity is mainly treated by a dietary therapy or an exercise therapy. However, in case of advanced obesity or in case that exercise is not available, a surgery (stomach contraction operation) or a medication (central nervous system stimulators such as an adrenergic drug, a serotonin-type drug, a digestive absorption inhibitor) have been employed, but such therapies are still in the trial stage, and an essential drug therapy for obesity has not been established yet.

Type 2 also includes a non-insulin dependent diabetes mellitus of the young people, i.e., MODY (maturity-onset type of the diabetes in the young), an insulin receptor abnormalities, or a diabetes induced by abnormalities of genes of enzymes or other molecules related with the glucose metabolism such as insulin secretion or insulin activity. Moreover, Type 2 also includes a morbid hyperglycemia being caused by continuous administration of a steroid drug such as glucocorticoid (a steroid diabetes), or a hyperglycemia of Cushing Syndrome or an acromegaly because they are diabetes under normal or high level of insulin conditions. Diabetes mellitus also includes other specific types of diabetes mellitus and gestational diabetes mellitus (see, Table I, above).

A “pharmaceutically effective amount” is intended an amount of a compound that, when administered to a subject or patient for preventing or treating a condition, disorder or disease, is sufficient to elicit a cellular response that is clinically significant, without excessive levels of side effects. See, “Formulations and Methods of Administration” section, infra, for further details.

“Subject” refers to any animal classified as a mammal, including humans, domestic and farm animals, and zoo, sports, and pet companion animals such as a household pet and other domesticated animal such as, but not limited to, cattle, sheep, ferrets, swine, horses, poultry, rabbits, goats, dogs, cats and the like. Preferred companion animals are dogs and cats. Preferably, the subject is human.

“Patient” refers to a subject, preferably a human, in need of treatment of a condition, disorder or disease, e.g., diabetes.

The terms “treat” and “treatment” refer to both therapeutic treatment and prophylactic or preventative measures, wherein the object is to prevent or slow down (lessen) an undesired physiological condition, disorder or disease or obtain beneficial or desired clinical results. For purposes of this invention, beneficial or desired clinical results include, but are not limited to, alleviation of symptoms; diminishment of extent of condition, disorder or disease; stabilized (i.e., not worsening) state of condition, disorder or disease; delay in onset or slowing of condition, disorder or disease progression; amelioration of the condition, disorder or disease state, remission (whether partial or total), whether detectable or undetectable; or enhancement or improvement of condition, disorder or disease. Treatment includes eliciting a cellular response that is clinically significant, without excessive levels of side effects. Treatment also includes prolonging survival as compared to expected survival if not receiving treatment.

Inhibitors of p38 MAP Kinase

As used herein, the term “inhibitor” includes, but is not limited to, any suitable molecule, compound, protein or fragment thereof, nucleic acid, formulation or substance that can regulate p38 MAP kinase activity. The inhibitor can affect a single p38 MAP kinase isoform (p38α, p38β, p38γ or p38δ), more than one isoform, or all isoforms of p38 MAP kinase. In a preferred embodiment, the inhibitor regulates the α isoform of p38 MAP kinase.

According to the present invention, it is contemplated that the inhibitor can exhibit its regulatory effect upstream or downstream of p38 MAP kinase or on p38 MAP kinase directly. Examples of inhibitor regulated p38 MAP kinase activity include those where the inhibitor can decrease transcription and/or translation of p38 MAP kinase, can decrease or inhibit post-translational modification and/or cellular trafficking of p38 MAP kinase, or can shorten the half-life of p38 MAP kinase. The inhibitor can also reversibly or irreversibly bind p38 MAP kinase, inactivate its enzymatic activity, or otherwise interfere with its interaction with downstream substrates.

If acting on p38 MAP kinase directly, in one embodiment the inhibitor should exhibit an IC₅₀ value of about 5 μM or less, preferably about 500 nM or less, more preferably about 100 nM or less. In a related embodiment, the inhibitor should exhibit an IC₅₀ value relative to the p38α MAP kinase isoform that is about ten fold less than that observed when the same inhibitor is tested against other p38 MAP kinase isoforms in a comparable assay.

Those skilled in the art can determine whether or not a compound is useful in the present invention by evaluating its p38 MAP kinase activity as well as its relative IC₅₀ value. This evaluation can be accomplished through conventional in vitro assays. In vitro assays include assays that assess inhibition of kinase or ATPase activity of activated p38 MAP kinase. In vitro assays can also assess the ability of the inhibitor to bind p38 MAP kinase or to reduce or block an identified downstream effect of activated p38 MAP kinase, e.g., cytokine secretion. IC₅₀ values are calculated using the concentration of inhibitor that causes a 50% decrease as compared to a control.

A binding assay is a fairly inexpensive and simple in vitro assay to run. As previously mentioned, binding of a molecule to p38 MAP kinase, in and of itself, can be inhibitory, due to steric, allosteric or charge-charge interactions. A binding assay can be performed in solution or on a solid phase using p38 MAP kinase or a fragment thereof as a target. By using this as an initial screen, one can evaluate libraries of compounds for potential p38 MAP kinase regulatory activity.

The target in a binding assay can be either free in solution, fixed to a support, or expressed in or on the surface of a cell. A label (e.g., radioactive, fluorescent, quenching, etc.) can be placed on the target, compound, or both to determine the presence or absence of binding. This approach can also be used to conduct a competitive binding assay to assess the inhibition of binding of a target to a natural or artificial substrate or binding partner. In any case, one can measure, either directly or indirectly, the amount of free label versus bound label to determine binding. There are many known variations and adaptations of this approach to minimize interference with binding activity and optimize signal.

For purposes of in vitro cellular assays, the compounds that represent potential inhibitors of p38 MAP kinase function can be administered to a cell in any number of ways. Preferably, the compound or composition can be added to the medium in which the cell is growing, such as tissue culture medium for cells grown in culture. The compound is provided in standard serial dilutions or in an amount determined by analogy to known modulators. Alternatively, the potential inhibitor can be encoded by a nucleic acid that is introduced into the cell wherein the cell produces the potential inhibitor itself.

Alternative assays involving in vitro analysis of potential inhibitors include those where cells (e.g., HeLa) transfected with DNA coding for relevant kinases can be activated with substances such as sorbitol, IL-1, TNF, or PMA. After immunoprecipitation of cell lysates, equal aliquots of immune complexes of the kinases are pre-incubated for an adequate time with a specific concentration of the potential inhibitor followed by addition of kinase substrate buffer mix containing labeled ATP and GST-ATF2 or MBP. After incubation, kinase reactions are terminated by the addition of SDS loading buffer. Phosphorylated substrate is resolved through SDS-PAGE and visualized and quantitated in a phosphorimager. The p38 MAP kinase regulation, in terms of phosphorylation and IC₅₀ values, can be determined by quantitation. See e.g., Kumar, S. et al., Biochem. Biophys. Res. Commun. 235:533-538 (1997).

Other in vitro assays can also assess the production of TNF-α as a correlation to p38 MAP kinase activity. One such example is a Human Whole Blood Assay. In this assay, venous blood is collected from, e.g., healthy male volunteers into a heparinized syringe and is used within 2 hours of collection. Test compounds are dissolved in 100% DMSO and 1 μl aliquots of drug concentrations ranging from 0 to 1 mM are dispensed into quadruplicate wells of a 24-well microtiter plate (Nunclon Delta SI, Applied Scientific Co., San Francisco, Calif.). Whole blood is added at a volume of 1 ml/well and the mixture is incubated for 15 minutes with constant shaking (Titer Plate Shaker, Lab-Line Instruments, Inc., Melrose Park, Ill.) at a humidified atmosphere of 5% CO₂ at 37° C. Whole blood is cultured either undiluted or at a final dilution of 1:10 with RPMI 1640 (Gibco 31800+NaHCO₃, Life Technologies, Rockville, Md. and Scios, Inc., Sunnyvale, Calif.). At the end of the incubation period, 10 μl of LPS (E. coli 0111:B4, Sigma Chemical Co., St. Louis, Mo.) is added to each well to a final concentration of 1 or 0.1 μg/ml for undiluted or 1:10 diluted whole blood, respectively. The incubation is continued for an additional 2 hours. The reaction is stopped by placing the microtiter plates in an ice bath, and plasma or cell-free supernates are collected by centrifugation at 3000 rpm for 10 minutes at 4° C. The plasma samples are stored at −80° C. until assayed for TNF-α levels by ELISA, following the directions supplied by Quantikine Human TNF-α assay kit (R&D Systems, Minneapolis, Minn.). IC₅₀ values are calculated using the concentration of inhibitor that causes a 50% decrease as compared to a control.

A similar assay is an Enriched Mononuclear Cell Assay. The enriched mononuclear cell assay begins with cryopreserved Human Peripheral Blood Mononuclear Cells (HPBMCs) (Clonetics Corp.) that are rinsed and resuspended in a warm mixture of cell growth media. The resuspended cells are then counted and seeded at 1×10⁶ cells/well in a 24-well microtitre plate. The plates are then placed in an incubator for an hour to allow the cells to settle in each well. After the cells have settled, the media is aspirated and new media containing 100 ng/ml of the cytokine stimulatory factor Lipopolysaccharide (LPS) and a test chemical compound is added to each well of the microtiter plate. Thus, each well contains HPBMCs, LPS and a test chemical compound. The cells are then incubated for 2 hours, and the amount of the cytokine Tumor Necrosis Factor Alpha (TNF-α) is measured using an Enzyme Linked Immunoassay (ELISA). One such ELISA for detecting the levels of TNF-α is commercially available from R&D Systems. The amount of TNF-α production by the HPBMCs in each well is then compared to a control well to determine whether the chemical compound acts as an inhibitor of cytokine production.

Compounds useful in the practice of the present invention include, but are not limited to, compounds of formula:

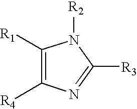

and pharmaceutically acceptable salts thereof,

wherein

R¹ is a heteroaryl ring selected from the group consisting of 4-pyridyl, pyrimidinyl, quinolyl, isoquinolinyl, quinazolin-4-yl, 1-imidazolyl, 1-benzimidazolyl, 4-pyridazinyl, and 1,2,4-triazin-5-yl, which heteroaryl ring is substituted one to three times with Y, N(R¹⁰)C(O)R_{b}, a halo-substituted mono- or di-C₁₋₆ alkyl-substituted amino, or NHRₐ, and which ring is further optionally substituted with C₁₋₄ alkyl, halogen, hydroxyl, optionally-substituted C₁₋₄ alkoxy, optionally-substituted C₁₋₄ alkylthio, optionally-substituted C₁₋₄ alkylsulfinyl, CH₂OR₁₂, amino, mono- or di-C₁₋₆ alkyl-substituted amino, NHRα, N(R₁₀)C(O)R_{b}, N(R₁₀)S(O)₂Rd, or an N-heterocyclyl ring which has from 5 to 7 members and optionally contains an additional heteroatom selected from the group consisting of oxygen, sulfur and NR₁₅;

Y is X₁—Rₐ;

X₁ is oxygen or sulfur;

Rₐ is C₁₋₆ alkyl, aryl, arylC₁₋₆ alkyl, heterocyclic, heterocyclylC₁₋₆ alkyl, heteroaryl or heteroarylC₁₋₆ alkyl, wherein each of these moieties is optionally substituted; R_{b} is hydrogen, C₁₋₆ alkyl, C₃₋₇ cycloalkyl, aryl, arylC₁₋₄ alkyl, heteroaryl, heteroarylC₁₋₄ alkyl, heterocyclyl or heterocyclylC₁₋₄ alkyl; Rd is C₁₋₆ alkyl, C₃₋₇ cycloalkyl, aryl, arylC₁₋₄ alkyl, heteroaryl, heteroarylC₁₋₄ alkyl, heterocyclyl or heterocyclylC₁₋₄ alkyl;

R₃ is hydrogen;

R₄ is phenyl, naphth-1-yl, naphth-2-yl or heteroaryl, which is optionally substituted by one or two substituents, each of which is independently selected, and which, for a 4-phenyl, 4-naphth-1-yl, 5-naphth-2-yl or 6-naphth-2-yl substituent, is halogen, cyano, nitro, —C(Z)NR₇R₁₇, —C(Z)OR₁₆, —(CR₁₀R₂₀)ᵥCOR₁₂, —SR₅, —SOR₅, —OR₁₂, halo-substituted-C₁₋₄ alkyl, C₁₋₄ alkyl, -ZC(Z)R₁₂, —NR₁₀C(Z)R₁₆ or —(CR₁₀R₂₀)ᵥNR₁₀R₂₀ and which, for other positions of substitution, is halogen, cyano, —C(Z)NR₁₃R₁₄, —C(Z)OR_{f}, —(CR₁₀R₂₀)_{m″}COR_{f}, —S(O)ₘR_{f}, —OR_{f}, —OR₁₂, halo-substituted C₁₋₄ alkyl, C₁₋₄ alkyl, —(CR₁₀R₂₀)_{m″}NR₁₀C(Z)R_{f}, —NR₁₀S(O)_{m′}R₈, —NR₁₀S(O)_{m′}NR₇R₁₇, -ZC(Z)R_{f}, -ZC(Z)R₁₂ or —(CR₁₀R₂₀)_{m″}NR₁₃R₁₄;

R_{f}is heterocyclyl, heterocyclylC₁₋₁₀ alkyl or R₈;

Z is oxygen or sulfur;

v is 0, 1 or 2;

m is 0, 1 or 2;

m′ is 1 or 2;

m″ is 0, 1, 2, 3, 4 or 5;

R₂ is C₁₋₁₀ alkyl N₃, —(CR₁₀R₂₀)_{n′}OR₉, heterocylyl, heterocycylC₁₋₁₀ alkyl, C₁₋₁₀ alkyl, halo-substituted C₁₋₁₀ alkyl, C₂₋₁₀ alkenyl, C₂₋₁₀ alkynyl, C₃₋₇ cycloalkyl, C₃₋₇ cycloalkylC₁₋₁₀alkyl, C₅₋₇ cycloalkenyl, C₅₋₇cycloalkenylC₁₋₁₀alkyl, aryl, arylC₁₋₁₀ alkyl, heteroaryl, heteroarylC₁₋₁₀alkyl, (CR₁₀R₂₀)ₙOR₁₁, (CR₁OR₂₀)ₙS(O)ₘR₁₈, (CR₁OR₂₀)ₙNHS(O)₂R₁₈, (CR₁₀R₂₀)ₙNR₁₃R₁₄, (CR₁₀R₂₀)ₙNO₂, (CR₁₀R₂₀)ₙCN, (CR₁₀R₂₀)_{n′}SO₂R₁₈, (CR₁₀R₂₀)ₙS(O)_{m′}NR₁₃R₁₄, (CR₁₀R₂₀)ₙC(Z)R₁₁, (CR₁₀R₂₀)ₙOC(Z)R₁₁, (CR₁₀R₂₀)ₙC(Z)OR₁₁, (CR₁₀R₂₀)ₙC(Z)NR₁₃R₁₄, (CR₁₀R₂₀)ₙC(Z)NR₁₀R₉, (CR₁₀R₂₀)ₙNR₁₀C(Z)R₁₁, (CR₁₀R₂₀)ₙNR₁₀C(Z)NR₁₃R₄, (CR₁₀R₂₀)ₙN(OR₆)C(Z)NR₁₃R₁₄, (CR₁₀R₂₀)ₙN(OR₆)C(Z)R₁₁, (CR₁₀R₂₀)ₙC(═NOR₆)R₁₁, (CR₁₀R₂₀)ₙNR₁₀C(═NR₁₉)NR₁₃R₁₄, (CR₁₀R₂₀)ₙOC(Z)NR₁₃R₁₄, (CR₁₀R₂₀)ₙNR₁₀C(Z)NR₁₃R₁₄, (CR₁₀R₂₀)ₙNR₁₀C(Z)OR₁₀, 5-(R₁₈)-1,2,4-oxadiazol-3-yl or 4-(R₁₂)-5-(R₁₈R₁₉)-4,5-dihydro-1,2,4-oxadiazol-3-yl; wherein the aryl, arylalkyl, heteroaryl, heteroaryl alkyl, cycloalkyl, cycloalkyl alkyl, heterocyclic and heterocyclic alkyl groups are optionally substituted;

n is an integer having a value of 1 to 10;

n′ is 0, or an integer having a value of 1 to 10;

R₅ is hydrogen, C₁₋₄ alkyl, C₂₋₄ alkenyl, C₂₋₄ alkynyl or NR₇R₁₇, excluding the moieties —SR₅ being —SNR₇R₁₇ and —S(O)R₅ being —SOH;

R₆ is hydrogen, a pharmaceutically-acceptable cation, C₁₋₁₀alkyl, C₃₋₇ cycloalkyl, aryl, arylC₁₋₄ alkyl, heteroaryl, heteroarylC₁₋₁₀alkyl, heterocyclyl, aroyl or C₁₋₁₀alkanoyl;

R₇ and R₁₇ are independently selected from the group consisting of hydrogen and C₁₋₄ alkyl, or R₇ and R₁₇ together with the nitrogen to which they are attached form a heterocyclic ring of 5 to 7 members which ring optionally contains an additional heteroatom selected from the group consisting of oxygen, sulfur and NR₁₅;

R₈ is C₁₋₁₀ alkyl, halo-substituted C₁₋₁₀ alkyl, C₂₋₁₀ alkenyl, C₂₋₁₀ alkynyl, C₃₋₇ cycloalkyl, C₅₋₇ cycloalkenyl, aryl, arylC₁₋₁₀alkyl, heteroaryl, heteroarylC₁₋₁₀ alkyl, (CR₁₀R₂₀)ₙOR₁₁, (CR₁₀R₂₀)ₙS(O)ₘR₁₈, (CR₁₀R₂₀)ₙNHS(O)₂R₁₈ or (CR₁₀R₂₀),NR₁₃R₁₄, wherein the aryl, arylalkyl, heteroaryl and heteroaryl alkyl are optionally substituted;

R₉ is hydrogen, —C(Z)R₁₁, optionally-substituted C₁₋₁₀alkyl, S(O)₂R₁₈, optionally-substituted aryl or optionally-substituted arylC₁₋₄ alkyl;

R₁₀ and R₂₀ are independently selected from the group consisting of hydrogen and C₁₋₄ alkyl;

R₁, is hydrogen, C₁₋₁₀ alkyl, C₃₋₇ cycloalkyl, heterocyclyl, heterocyclylC₁₋₁₀alkyl, aryl, arylC₁₋₁₀ alkyl, heteroaryl or heteroarylC₁₋₁₀ alkyl;

R₁₂ is hydrogen or R₁₆;

R₁₃ and R₁₄ are independently selected from the group consisting of hydrogen, optionally-substituted C₁₋₄ alkyl, optionally-substituted aryl and optionally-substituted arylC₁₋₄ alkyl, or together with the nitrogen to which they are attached form a heterocyclic ring of 5 to 7 members, which ring optionally contains an additional heteroatom selected from the group consisting of oxygen, sulfur and NR₉;

R₁₅ is R₁₀ or C(Z)C₁₋₄ alkyl;

R₁₆ is C₁₋₄ alkyl, halo-substituted C₁₋₄ alkyl or C₃₋₇ cycloalkyl;

R₁₈ is C₁₋₁₀alkyl, C₃₋₇ cycloalkyl, heterocyclyl, aryl, arylC₁₋₁₀alkyl, heterocyclyl, heterocyclylC₁₋₁₀ alkyl, heteroaryl or heteroarylC₁₋₁₀ alkyl; and

R₁₉ is hydrogen, cyano, C₁₋₄ alkyl, C₃₋₇ cycloalkyl or aryl; or wherein

R₁, Y, X₁, Rₐ, R_{b}, R_{d}, V, m, m′, m, Z, n, n′ and R₅ are defined as above, and R₂ is hydrogen, C₁₋₁₀ alkyl, halo-substituted C₁₋₁₀alkyl, C₂₋₁₀ alkenyl, C₂₋₁₀ alkynyl, C₃₋₇ cycloalkyl, C₃₋₇ cycloalkylC₁₋₁₀alkyl, C₅₋₇ cycloalkenyl, aryl, arylC₁₋₁₀alkyl, heteroaryl, heteroarylC₁₋₁₀alkyl, heterocyclyl, heterocyclylC₁₋₁₀ alkyl, (CR₁₀R₂₈)_{n′}OR₁₂, (CR₁₀R₂₈)_{n′}OR₁₃, (CR₁₀R₂₈)_{n′}S(O)ₘR₂₅, (CR₁₀R₂₈)_{n′}S(O)₂R₂₅, (CR₁₀R₂₈)_{n′}NHS(O)₂R₂₅, (CR₁₀R₂₈)_{n′}NR₈R₉, (CR₁₀R₂₈)_{n′}NO₂, (CR₁₀R₂₈)_{n′}CN, (CR₁₀R₂₈)_{n′}S(O)ₙNR₈R₉, (CR₁₀R₂₈)ₙC(Z)R₁₃, (CR₁₀R₂₈)_{n′}C(Z)OR₁₃, (CR₁₀R₂₈)_{n′}C(Z)NR₈R₉, (CR₁₀R₂₈)_{n′}C(Z)NR₁₃OR₁₂, (CR₁₀R₂₈)_{n′}NR₁₀C(Z)R₁₃, (CR₁₀R₂₈)_{n′}NR₁₀C(Z)NR₈R₉, (CR₁₀R₂₈)_{n′}N(OR₂₁)C(Z)NR₈R₉, (CR₁₀R₂₈)_{n′}N(OR₂₈)C(Z)R₁₃, (CR₁₀R₂₈)_{n′}C(═NOR₂₁)R₁₃, (CR₁₀R₂₈)_{n′}NR₁₀C(═NR₂₇)NR₈R₉, (CR₁₀R₂₈)_{n′}OC(Z)NR₈R₉, (CR₁₀R₂₈)_{n′}NR₁₀C(z)OR₁₀, (CR₁₀R₂₈)_{n′}NR₁₀C(Z)OR₁₀, 5-(R₂₅)-1,2,4-oxadiazol-3-yl or 4-(R₁₂)-5-(R₁₈R₁₉)-4,5-dihydro-1,2,4-oxadiazol-3-yl, wherein the cycloalkyl, cycloalkyl alkyl, aryl, arylalkyl, heteroaryl, heteroarylalkyl, heterocyclyl and heterocyclylalkyl moieties are optionally substituted;

R₃ is hydrogen or Q-(Y₁)ₜ;

Q is an aryl or heteroaryl group;

t is 1, 2 or 3;

Y₁ is independently selected from the group consisting of hydrogen, C₁₋₅ alkyl, halo-substituted C₁₋₅ alkyl, halogen and —(CR₁₀R₂₀)ₙY₂;

Y₂ is OR₈, NO₂, S(O)ₘ″R₁₁, SR₈, S(O)ₘ″OR₈, S(O)ₘNR₈R₉, NR₈R₉, O(CR₁₀R₂₀)_{n′}NR₈R₉, C(O)R₈, CO₂R₈, CO₂(CR₁₀R₂₀)ₙ′CONR₈R₉, ZC(O)R₈, CN, C(Z)NR₈R₉, NR₁₀C(Z)R₈, C(Z)NR₈OR₉, NR₁₀C(Z)NR₈R₉, NR₁₀S(O)ₘ″R₁₁, N(OR₂₁)C(Z)NR₈R₉, N(OR₂₁)C(Z)R₈, C(═NOR₂₈)R₈, NR₁₀C(═NR₁₅)SR₁₁, NR₁₀C(═NR₁₅)NR₈R₉, NR₁₀C(═CR₁₄R₂₄)SR₁₁, NR₁₀C(═CR₁₄R₂₄)NR₈R₉, NR₁₀C(O)C(O)NR₈R₉, NR₁₀C(O)C(O)OR₁₀, C(═NR₁₃)NR₈R₉, C(═NOR₁₃)NR₈R₉, C(═NR₁₃)ZR₁₀, C(Z)NR₈R₉, NR₁₀S(O)ₘ″CF₃NR₁₀C(Z)OR₁₀, 5-(R₁₈)-1,2,4-oxadiazol-3-yl or 4-(R₁₂)-5-(R₁₈R₁₉)-4,5-dihydro-1,2,4-oxadiazol-3-yl;

R₄ is phenyl, naphth-1-yl or naphth-2-yl, which is optionally substituted by one or two substituents, each of which is independently selected, and which, for a 4-phenyl, 4-naphth-1-yl or 5-naphth-2-yl substituent, is halo, nitro, cyano, C(Z)NR₇R₁₇, C(Z)OR₂₃, (CR₁₀R₂₀)ᵥCOR₃₆, SR₅, SOR₅, OR₃₆, halo-substituted-C₁₋₄ alkyl, C₁₋₄ alkyl, ZC(Z)R₃₆, NR₁₀C(Z)R₂₃ or (CR₁₀R₂₀)ₙNR₁₀R₂₀, and which, for other positions of substitution, is halo, nitro, cyano, C(Z)NR₁₆R₂₆, C(Z)OR₈, (CR₁₀R₂₀)ₘ″COR₈, S(O)ₘR₈, OR₈, halo-substituted-C₁₋₄ alkyl, C₁₋₄ alkyl, (CR₁₀R₂₀)ₘ″NR₁₀C(Z)R₈NR₁₀S(O)_{m′}R₁₁, NR₁₀S(O)ₘ′NR₇R₁₇, ZC(Z)R₈ or (CR₁₀R₂₀)ₘ″NR₁₆R₂₆;

R₇ and R₁₇ are independently selected from the group consisting of hydrogen and C₁₋₄ alkyl, or R₇ and R₁₇ together with the nitrogen to which they are attached form a heterocyclic ring of 5 to 7 members, which ring optionally contains an additional heteroatom selected from the group consisting of oxygen, sulfur and NR₂₂;

R₈ is hydrogen, heterocyclyl, heterocyclylalkyl or R₁₁;

R₉ is hydrogen, C₁₋₁₀alkyl, C₂₋₁₀ alkenyl, C₂₋₁₀ alkynyl, C₃₋₇ cycloalkyl, C₅₋₇ cycloalkenyl, aryl, arylalkyl, heteroaryl or heteroarylalkyl, or R₈ and R₉ can together with the nitrogen to which they are attached form a heterocyclic ring of 5 to 7 members, which ring optionally contains an additional heteroatom selected from the group consisting of oxygen, sulfur and NR₁₂;

R₁₀ and R₂₀ are independently selected from the group consisting of hydrogen and C₁₋₄ alkyl;

R₁₁ is C₁₋₁₀alkyl, halo-substituted C₁₋₁₀alkyl, C₂₋₁₀ alkenyl, C₂₋₁₀ alkynyl, C₃₋₇ cycloalkyl, C₅₋₇ cycloalkenyl, aryl, arylalkyl, heteroaryl or heteroarylalkyl;

R₁₂ is hydrogen, —C(Z)R₁₃, optionally-substituted C₁₋₄ alkyl, optionally-substituted aryl, optionally-substituted arylC₁₋₄ alkyl or S(O)₂R₂₅;

R₁₃ is hydrogen, C₁₋₁₀alkyl, C₃₋₇ cycloalkyl, heterocyclyl, heterocyclylC₁₋₁₀ alkyl, aryl, arylC₁₋₁₀alkyl, heteroaryl or heteroaryl C₁₋₁₀ alkyl, wherein all of these moieties are optionally substituted;

R₁₄ and R₂₄ are independently selected from the group consisting of hydrogen, alkyl, nitro and cyano;

R₁₅ is hydrogen, cyano, C₁₋₄ alkyl, C₃₋₇ cycloalkyl or aryl;

R₁₆ and R₂₆ are independently selected from the group consisting of hydrogen, optionally-substituted C₁₋₄ alkyl, optionally-substituted aryl and optionally-substituted arylC₁₋₄ alkyl, or together with the nitrogen to which they are attached form a heterocyclic ring of 5 to 7 members, which ring optionally contains an additional heteroatom selected from the group consisting of oxygen, sulfur and NR₁₂;

R₁₈ and R₁₉ are independently selected from the group consisting of hydrogen, C₁₋₄ alkyl, substituted alkyl, optionally-substituted aryl and optionally-substituted arylalkyl, or together denote an oxygen or sulfur;

R₂₁ is hydrogen, a pharmaceutically-acceptable cation, C₁₋₁₀ alkyl, C₃₋₇ cycloalkyl, aryl, arylC₁₋₄ alkyl, heteroaryl, heteroarylalkyl, heterocyclyl, aroyl or C₁₋₁₀ alkanoyl;

R₂₂ is R₁₀ or C(Z)-C₁₋₄ alkyl;

R₂₃ is C₁₋₄ alkyl, halo-substituted-C₁₋₄ alkyl or C₃₋₅ cycloalkyl;

R₂₅ is C₁₋₁₀ alkyl, C₃₋₇ cycloalkyl, heterocyclyl, aryl, arylalkyl, heterocyclyl, heterocyclylC₁₋₁₀alkyl, heteroaryl or heteroarylalkyl;

R₂₇ is hydrogen, cyano, C₁₋₄ alkyl, C₃₋₇ cycloalkyl or aryl;

R₂₈ is hydrogen, C₁₋₆ alkyl, C₃₋₇ cycloalkyl, aryl, arylC₁₋₄ alkyl, heteroaryl, heteroarylC₁₋₄alkyl, heterocyclyl or heterocyclylC₁₋₄ alkyl, all of which are optionally substituted; and

R₃₆ is hydrogen or R₂₃.

Exemplary compounds of this formula include:

1-[3-(4-morpholinyl)propyl]-4-(4-fluorophenyl)-5-(4-pyridyl)imidazole;

1-(3-chloropropyl)-4-(4-fluorophenyl)-5-(4-pyridyl)imidazole;

1-(3-azidopropyl)-4-(4-fluorophenyl)-5-(4-pyridyl)imidazole;

1-(3-aminopropyl)-4-(4-fluorophenyl)-5-(4-pyridyl)imidazole;

1-(3-methylsulfonamidopropyl)-4-(4-fluorophenyl)-5-(4-pyridyl)imidazole;

1-[3-(N-phenylmethyl)aminopropyl]-4-(4-fluorophenyl)-5-(4-pyridyl)imidazole;

1-[3-(N-phenylmethyl-N-methyl)aminopropyl]-4-(4-fluorophenyl)-5-(4-pyridyl)imidazole;

1-[3-(1-pyrrolidinyl)propyl]-4-(4-fluorophenyl)-5-(4-pyridyl)imidazole;

1-(3-diethylaminopropyl)-4-(4-fluorophenyl)-5-(4-pyridyl)imidazole;

1-[3-(1-piperidinyl)propyl]-4-(4-fluorophenyl)-5-(4-pyridyl)imidazole;

1-[3-(methylthio)propyl]-4-(4-fluorophenyl)-5-(4-pyridyl)imidazole;

1-[2-(4-morpholinyl)ethyl]-4-(4-fluorophenyl)-5-(4-pyridyl)imidazole;

1-[3-(4-morpholinyl)propyl]-4-(3-methylthiophenyl)-5-(4-pyridyl)imidazole;

(+/−)-1-[3-(4-morpholinyl)propyl]-4-(3-methylsulfinylphenyl)-5-(4-pyridyl)imidazole; 1-[3-(N-methyl-N-benzyl)aminopropyl]-4-(3-methylthiophenyl)-5-(4-pyridyl)imidazole;

1-[3-(N-methyl-N-benzyl)aminopropyl]-4-(3-methylsulfinylphenyl)-5-(4-pyridyl)imidazole;

1-[4-(methylthio)phenyl]-4-(4-fluorophenyl)-5-(4-pyridyl)imidazole;

1-[4-(methylsulfinyl)phenyl]-4-(4-fluorophenyl)-5-(4-pyridyl)imidazole;

1-[3-(methylthio)phenyl]-4-(4-fluorophenyl)-5-(4-pyridyl)imidazole;

(+/−)-1-[3-(methylsulfinyl)phenyl]-4-(4-fluorophenyl)-5-(4-pyridyl)imidazole;

1-[2-(methylthio)phenyl]-4-(4-fluorophenyl)-5-(4-pyridyl)imidazole;

1-[2-(methylsulfinyl)phenyl]-4-(4-fluorophenyl)-5-(4-pyridyl)imidazole;

1-[4-(4-morpholinyl)butyl]-4-(4-fluorophenyl)-5-(4-pyridyl)imidazole;

1-cyclopropyl-4-(4-fluorophenyl)-5-(4-pyridyl)imidazole;

1-isopropyl-4-(4-fluorophenyl)-5-(4-pyridyl)imidazole;

1-cyclopropylmethyl-4-(4-fluorophenyl)-5-(4-pyridyl)imidazole;

1-tert-butyl-4-(4-fluorophenyl)-5-(4-pyridyl)imidazole;

1-(2,2-diethoxyethyl)-4-(4-fluorophenyl)-5-(4-pyridyl)imidazole;

1-formylmethyl-4-(4-fluorophenyl)-5-(4-pyridyl)imidazole;

1-hydroxyiminylmethyl-4-(4-fluorophenyl)-5-(4-pyridyl)imidazole;

1-cyanomethyl-4-(4-fluorophenyl)-5-(4-pyridyl)imidazole;

1-[3-(4-morpholinyl)propyl]-4-(4-fluorophenyl)-5-(2-methylpyrid-4-yl)imidazole;

4-(4-fluorophenyl)-1-[3-(4-morpholinyl)propyl]-5-(2-chloropyridin-4-yl)imidazole;

4-(4-fluorophenyl)-1-[3-(4-morpholinyl)propyl]-5-(2-amino-4-pyridinyl)imidazole;

1-(4-carboxymethyl)propyl-4-(4-fluorophenyl)-5-(4-pyridyl)imidazole;

1-(4-carboxypropyl)-4-(4-fluorophenyl)-5-(4-pyridyl)imidazole;

1-(3-carboxymethyl)ethyl-4-(4-fluorophenyl)-5-(4-pyridyl)imidazole;

1-(3-carboxy)ethyl-4-(4-fluorophenyl)-5-(4-pyridyl)imidazole;

1-(1-benzylpiperidin-4-yl)-4-(4-fluorophenyl)-5-(4-pyridyl)imidazole;

5-(2-aminopyrimidin-4-yl)-4-(4-fluorophenyl)-1-[3-(4-morpholinyl)propyl]imidazole;

5-(2-aminopyrimidin-4-yl)-4-(4-fluorophenyl)-1-(1-benzylpiperidin-4-yl)imidazole;

5-(2-aminopyrimidin-4-yl)-4-(4-fluorophenyl)-1-(2-propyl)imidazole;

5-(2-aminopyrimidin-4-yl)-4-(4-fluorophenyl)-1-(cyclopropylmethyl)imidazole;

5-(2-aminopyrimidin-4-yl)-4-(4-fluorophenyl)-1-(1-carboxyethyl-4-piperidinyl)imidazole;

5-(2-aminopyrimidin-4-yl)-4-(4-fluorophenyl)-1-(4-piperidinyl)imidazole;

1-methyl-4-phenyl-5-(4-pyridyl)imidazole; 1-methyl-4-[3-(chlorophenyl)]-5-(4-pyridinyl)imidazole;

1-methyl-4-(3-methylthiophenyl)-5-(4-pyridyl)imidazole;

(+/−)-1-methyl-4-(3-methylsulfinylphenyl)-5-(4-pyridyl)imidazole;

(+/−)-4-(4-fluorophenyl)-1-[3-(methylsulfinyl)propyl]-5-(4-pyridinyl)imidazole;

4-(4-fluorophenyl)-1-[(3-methylsulfonyl)propyl]-5-(4-pyridinyl)imidazole; 41-(3-phenoxypropyl)-4-(4-fluorophenyl)-5-(4-pyridinyl)imidazole;

1-[3-(phenylthio)propyl]-4-(4-fluorophenyl)-5-(4-pyridinyl)imidazole;

1-[3-(4-morpholinyl)propyl]-4-(4-fluorophenyl)-5-(4-quinolyl)imidazole;

(+/−)-1-(3-phenylsulfinylpropyl)-4-(4-fluorophenyl)-5-(4-pyridinyl)imidazole;

1-(3-ethoxypropyl)-4-(4-fluorophenyl)-5-(4-pyridinyl)imidazole;

1-(3-phenylsulfonylpropyl-4-(4-fluorophenyl)-5-(4-pyridinyl)imidazole;

1-[3-(4-morpholinyl)propyl]-4-(3-chlorophenyl)-5-(4-pyridyl)imidazole;

1-[3-(4-morpholinyl)propyl]-4-(3,4-dichlorophenyl)-5-(4-pyridyl)imidazole;

4-[4-(4-fluorophenyl)-1-[3-(4-morpholinyl)propyl]-5-(pyrimid-2-one-4-yl)imidazole;

4-(4-fluorophenyl)-5-[2-(methylthio)-4-pyrimidinyl]-1-[3-(4-morpholinyl)propyl]imidazole;

(+/−)-4-(4-fluorophenyl)-5-[2-(methylsulfinyl)-4-pyrimidinyl]-1-[3-(4-morpholinyl)propyl]imida zole;

(E)-1-(1-propenyl)-4-(4-fluorophenyl)-5-(4-pyridinyl)imidazole;

1-(2-propenyl)-4-(4-fluorophenyl)-5-(4-pyridinyl)imidazole;

5-[(2-N,N-dimethylamino)pyrimidin-4-yl]-4-(4-fluorophenyl)-1-[3-(4-morpholinyl)propyl]imida zole;

1-[3-(4-morpholinyl)propyl]-5-(4-pyridinyl)-4-[4-(trifluoromethyl)phenyl]imidazole;

1-[3-(4-morpholinyl)propyl]-5-(4-pyridinyl)-4-[3-(trifluoromethyl)phenyl]imidazole;

1-(cyclopropylmethyl)-4-(3,4-dichlorophenyl)-5-(4-pyridinyl)imidazole;

1-(cyclopropylmethyl)-4-(3-trifluoromethylphenyl)-5-(4-pyridinyl)imidazole;

1-(cyclopropylmethyl)-4-(4-fluorophenyl)-5-(2-methylpyrid-4-yl)imidazole;

1-[3-(4-morpholinyl)propyl]-5-(4-pyridinyl)-4-(3,5-bistrifluoromethylphenyl)imidazole;

5-[4-(2-aminopyrimidinyl)]-4-(4-fluorophenyl)-1-(2-carboxy-2,2-dimethylethyl)imidazole;

1-(1-formyl-4-piperidinyl)-4-(4-fluorophenyl)-5-(4-pyridinyl)imidazole;

5-(2-amino-4-pyrimidinyl)-4-(4-fluorophenyl)-1-(1-methyl-4-piperidinyl)imidazole;

1-(2,2-dimethyl-3-morpholin-4-yl)propyl-4-(4-fluorophenyl)-5-(2-amino-4-pyrimidinyl)imidazole;

4-(4-fluorophenyl)-5-(4-pyridyl)-1-(2-acetoxyethyl)imidazole;

5-(2-aminopyrimidin-4-yl)-4-(4-fluorophenyl)-1-(1-benzylpyrrolin-3-yl)imidazole;

5-(2-aminopyrimidin-4-yl)-4-(4-fluorophenyl)-1-(2,2,6,6-tetramethylpiperidin-4-yl)imidazole;

5-[4-(2-N-methylamino)pyrimidinyl]-4-(4-fluorophenyl)-1-(4-N-methylpiperidine)imidazole;

5-[4-(2-N-methylamino)pyrimidinyl]-4-(4-fluorophenyl)-1-(4-N-morpholino-1-propyl)imidazole;

5-[4-(2-N-methylamino)pyrimidinyl)-4-(4-fluorophenyl)-1-(4-piperidine)imidazole;

5-[(2-ethylamino)pyrimidin-4-yl]-4-(4-fluorophenyl)-1-(1-methylpiperidin-4-yl)imidazole;

4-(4-fluorophenyl)-5-[2-(isopropyl)aminopyrimidin-4-yl]-1-(1-methylpiperidin-4-yl)imidazole;

5-(2-acetamido-4-pyrimidinyl)-4-(4-fluorophenyl)-1-(4-N-morpholino-1-propyl)imidazole;

5-(2-acetamido-4-pyrimidinyl)-4-(4-fluorophenyl)-1-(1-methyl-4-piperidinyl)imidazole;

5-[4-(2-N-methylthio)pyrimidinyl]-4-(4-fluorophenyl)-1-(4-piperidine)imidazole;

4-(fluorophenyl)-1-(methyl-4-piperidinyl)-5-(2-methylthio-4-pyrimidinyl)imidazole;

4-(fluorophenyl)-1-(methyl-4-piperidinyl)-5-(2-methysulfinyl-4-pyrimidinyl)imidazole;

1-tert-butyl-4-(4-fluorophenyl)-5-(2-methysulfinyl-4-pyrimidinyl)imidazole;

5-[4-(2-aminopyrimidinyl)]-4-(4-fluorophenyl)-1-(2,2,6,6-tetramethyl-4-piperidinyl)imidazole;

5-[4-(2-N-methylamino-4-pyrimidinyl)]-4-(4-fluorophenyl)-1-(2,2,6,6-tetramethyl-4-piperidine)imidazole;

5-(2-amino-4-pyrimidinyl)-4-(4-fluorophenyl)-1-(tetrahydro-4-thiopyranyl)imidazole;

5-(2-amino-4-pyrimidinyl)-4-(4-fluorophenyl)-1-(tetrahydro-4-pyranyl)imidazole;

5-(2-methylamino-4-pyrimidinyl)-4-(4-fluorophenyl)-1-(2-cyanoethyl)imidazole;

5-(2-amino-4-pyrimidinyl)-4-(4-fluorophenyl)-1-(tetrahydro-4-sulfinylpyranyl)imidazole;

5-(2-amino-4-pyrimidinyl)-4-(4-fluorophenyl)-1-(tetrahydro-4-sulfonylpyranyl)imidazole;

5-(2-methylamino-4-pyrimidinyl)-4-(4-fluorophenyl)-1-(2,2,2-trifluoroethyl-4-piperidinyl)imidazole;

5-(2-amino-4-pyrimidinyl)-4-(4-fluorophenyl)-1-(trifluoroacetyl-4-piperidinyl)imidazole;

5-(4-pyridyl)-4-(4-fluorophenyl)-1-(4-piperidinyl)imidazole;

5-(4-pyridyl)-4-(4-fluorophenyl)-1-(1-t-butoxycarbonyl-4-piperidinyl)imidazole;

5-(2-amino-4-pyrimidinyl)-4-(4-fluorophenyl)-1-(4-(1,3-dioxycyclopentyl)cyclohexyl) imidazole;

5-(2-amino-4-pyrimidinyl)-4-(4-fluorophenyl)-1-(4-ketocyclohexyl)imidazole;

5-(2-amino-4-pyrimidinyl)-4-(4-fluorophenyl)-1-(4-cyclohexyl oxime) imidazole;

5-(2-amino-4-pyrimidinyl)-4-(4-fluorophenyl)-1-(4-cyclohexyl hydroxylamine) imidazole;

5-(2-amino-4-pyrimidinyl)-4-(4-fluorophenyl)-1-(trans-4-hydroxyurea) imidazole;

5-(2-amino-4-pyrimidinyl)-4-(4-fluorophenyl)-1-(cis-4-hydroxyurea) imidazole;

5-(2-amino-4-pyrimidinyl)-4-(4-fluorophenyl)-1-(4-hydroxycyclohexyl)imidazole;

5-[4-(2-N-methylamino)pyrimidinyl]-4-(4-fluorophenyl)-1-(4-ketocyclohexyl)imidazole;

5-[4-(2-N-methylamino)pyrimidinyl]-4-(4-fluorophenyl)-1-(trans-4-hydroxycyclohexyl)imidazole;

5-[4-(2-N-methylamino)pyrimidinyl]-4-(4-fluorophenyl)-1-(cis-4-hydroxycyclohexyl)imidazole;

5-[4-(2-N-methylamino)pyrimidinyl]-4-(4-fluorophenyl)-1-[4-(cis-pyrrolidinyl)cyclohexyl]imidazole;

5-[4-(2-N-methylamino)pyrimidinyl]-4-(4-fluorophenyl)-1-[4-(trans-1-pyrrolidinyl)cyclohexyl]imidazole;

5-[4-(2-N-methylamino)pyrimidinyl]-4-(4-fluorophenyl)-1-(4-ethynyl-4-hydroxycyclohexyl)imidazole;

5-[4-(2-N-methylamino)pyrimidinyl]-4-(4-fluorophenyl)-1-(4-(1-propynyl)-4-hydroxycyclohexyl)imidazole;

5-[4-(2-N-methylamino)pyrimidinyl]-4-(4-fluorophenyl)-1-(4-amino-4-methylcyclohexyl)imidazole;

5-[4-(2-N-methylamino)pyrimidinyl]-4-(4-fluorophenyl)-1-(4-acetamido-4-methylcyclohexyl)imidazole;

5-[4-(2-N-methylamino)pyrimidinyl]-4-(4-fluorophenyl)-1-(4-hydroxy-4-methylcyclohexyl)imidazole;

5-[4-(2-N-methylamino)pyrimidinyl]-4-(4-fluorophenyl)-1-(4-oxiranylcyclohexyl)imidazole;

5-[4-(2-N-methylamino)pyrimidinyl]-4-(4-fluorophenyl)-1-(4-cyanomethyl-4-hydroxycyclohexyl)imidazole;

5-[4-(2-N-methylamino)pyrimidinyl]-4-(4-fluorophenyl)-1-(4-hydroxy-4-hydroxymethylcyclohexyl)imidazole;

5-[4-(2-amino)pyrimidinyl]-4-(4-fluorophenyl)-1-[4-hydroxy-4-(1-propynyl)-cyclohexyl]imidazole;

5-[4-(2-amino)pyrimidinyl]-4-(4-fluorophenyl)-1-(4-hydroxy-4-methylcyclohexyl)imidazole;

5-[4-(2-N-methylamino)pyrimidinyl]-4-(4-fluorophenyl)-1-(4-hydroxy-4-isopropyl-cyclohexyl)imidazole;

5-[4-(2-N-methylamino)pyrimidinyl]-4-(4-fluorophenyl)-1-(4-hydroxy-4-phenyl-cyclohexyl)imidazole;

5-[4-(2-N-methylamino)pyrimidinyl]-4-(4-fluorophenyl)-1-(4-hydroxy-4-benzyl-cyclohexyl)imidazole;

5-[4-(2-N-methylamino)pyrimidinyl]-4-(4-fluorophenyl)-1-(4-hydroxy-4-cyanomethyl cyclohexyl)imidazole;

5-[4-(2-N-methylamino)pyrimidinyl]-4-(4-fluorophenyl)-1-(4-hydroxy-4-(2-cyanoethyl)cyclohexyl)imidazole;

5-[4-(2-N-methylamino)pyrimidinyl]-4-(4-fluorophenyl)-1-(4-hydroxy-4-(2-aminoethyl)cyclohexyl)imidazole;

5-[4-(2-N-methylamino)pyrimidinyl]-4-(4-fluorophenyl)-1-(4-hydroxy-4-(2-nitroethyl)-cyclohexyl)imidazole;

5-[4-(2-N-methylamino)pyrimidinyl]-4-(4-fluorophenyl)-1-(4-hydroxymethyl-4-amino-cyclohexyl)imidazole;

5-[4-(2-N-methylamino)pyrimidinyl]-4-(4-fluorophenyl)-1-(4-hydroxy-4-amino-cyclohexyl)imidazole;

5-[4-(2-N-methylamino)pyrimidinyl]-4-(4-fluorophenyl)-1-(4-aminocyclohexyl)imidazole;

5-[4-(2-N-methylamino)pyrimidinyl]-4-(4-fluorophenyl)-1-(4-hydroxy-4-thiomethyl cyclohexyl)imidazole;

5-[4-(2-N-methylamino)pyrimidinyl]-4-(4-fluorophenyl)-1-(4-hydroxy-4-hydroxy methylcyclohexyl)imidazole;

5-[4-(2-N-methylamino)pyrimidinyl]-4-(4-fluorophenyl)-1-(4-hydroxy-4-aminomethylcyclohexyl)imidazole;

5-[4-(2-amino)pyrimidinyl]-4-(4-fluorophenyl)-1-(4-amino-4-methyl-cyclohexyl)imidazole;

5-[4-(2-amino)pyrimidinyl]-4-(4-fluorophenyl)-1-(4-hydroxy-4-methyl-cyclohexyl)imidazole;

5-[4-(2-amino)pyrimidinyl]-4-(4-fluorophenyl)-1-(4-oxiranylcyclohexyl)imidazole;

4-(fluorophenyl)-1-(methyl-4-piperidinyl)-5-(2-methysulfinyl-4-pyrimidinyl)imidazole;

4-(fluorophenyl)-1-(methyl-4-piperidinyl)-5-(2-methylthio-4-pyrimidinyl)imidazole;

5-[(2-benzylamino)pyrimidin-4-yl]-4-(4-fluorophenyl)-1-(1-methylpiperidin-4-yl)imidazole;

4-(4-fluorophenyl)-1-(1-methylpiperidin-4-yl)-5-[2-(4-tetrahydrothiopyranyl)aminopyrimidin-4-yl]imidazole;

4-(4-fluorophenyl)-5-[(2-hydroxy)ethylamino]pyrimidin-4-yl-1-(1-methylpiperidin-4-yl)imidazole;

5-[(2-(3-chlorobenzylamino)pyrimidin-4-yl]-4-(4-fluorophenyl)-1-(1-methylpiperidin-4-yl)imidazole;

5-[(2-(1-naphthylmethylamino)pyrimidin-4-yl]-4-(4-fluorophenyl)-1-(1-methylpiperidin-4-yl)imidazole;

5-[(2-(1-benzyl-4-piperidinylamino)pyrimidin-4-yl]-4-(4-fluorophenyl)-1-(1-methylpiperidin-4-yl)imidazole;

4-(4-fluorophenyl)-1-(1-methylpiperidin-4-yl)-5-[2-[3-(morpholino)propyl]aminopyrimidin-4-yl]imidazole;

5-[2-[(3-bromophenyl)amino]pyrimidin-4-yl]-4-(4-fluorophenyl)-1-(-methylpiperidin-4-yl)imidazole;

5-[(2-(piperonylamino)pyrimidin-4-yl]-4-(4-fluorophenyl)-1-(1-methylpiperidin-4-yl)imidazole;

5-[(2-(4-piperidinylamino)pyrimidin-4-yl]-4-(4-fluorophenyl)-1-(1-methylpiperidin-4-yl)imidazole;

5-[(2-(5-chlorotryptamino)pyrimidin-4-yl]-4-(4-fluorophenyl)-1-(1-methylpiperidin-4-yl)imidazole;

5-[(2-(2,2,6,6-tetramethylpiperidin-4-yl)aminopyrimidin-4-yl]-4-(4-fluorophenyl)-1-(1-methylpiperidin-4-yl)imidazole;

5-[(2-[1-ethoxycarbonyl)piperidin-4-yl]aminopyrimidin-4-yl]-4-(4-fluorophenyl)-1-(1-methylpiperidin-4-yl)imidazole;

1-(4-oxocyclohexyl)-4-(4-fluorophenyl)-5-[(2-methoxy)pyrimidin-4-y]imidazole;

cis-1-(4-hydroxycyclohexyl)-4-(4-fluorophenyl)-5-[(2-methoxy)pyrimidin-4-yl]imidazole;

trans-1-(4-hydroxycyclohexyl)-4-(4-fluorophenyl)-5-[(2-methoxy)pyrimidin-4-yl]imidazole;

1-(4-oxocyclohexyl)-4-(4-fluorophenyl)-5-[(2-methylthio)pyrimidin-4-yl]imidazole;

trans-1-(4-hydroxycyclohexyl)-4-(4-fluorophenyl)-5-[(2methylthio)pyrimidin-4-yl]imidazole;

1-(4-oxocyclohexyl)-4-(4-fluorophenyl)-5-[(2-hydroxy)pyrimidin-4-yl]imidazole;

1-(4-oxocyclohexyl)-4-(4-fluorophenyl)-5-[(2-isopropoxy)pyrimidin-4-yl]imidazole;

1-(4-hydroxycyclohexyl)-4-(4-fluorophenyl)-5-[(2-isopropoxy)pyrimidin-4-yl]imidazole;

trans-1-(4-hydroxy-4-methylcyclohexyl)-4-(4-fluorophenyl)-5-[(2-methoxy)pyrimidin-4-yl]imidazole;

cis-1-(4-hydroxy-4-methylcyclohexyl)-4-(4-fluorophenyl)-5-[(2-methoxy)pyrimidin-4-yl]imidazole;

trans-1-(4-hydroxycyclohexyl)-4-(4-fluorophenyl)-5-[(2-ethoxy)pyrimidin-4-yl]imidazole;

1-(4-piperidinyl)-4-(4-fluorophenyl)-5-(2-phenoxypyrimidin-4-yl)imidazole;

1-(4-piperidinyl)-4-(4-fluorophenyl)-5-(2-phenoxy-4-pyridinyl)imidazole;

1-(4-piperidinyl)-4-(4-fluorophenyl)-5-[2-(4-methoxyphenoxy)-4-pyridinyl]imidazole;

1-(4-piperidinyl)-4-(4-fluorophenyl)-5-[2-(4-fluorophenoxy)-4-pyridinyl]imidazole;

1-(piperidin-4-yl)-4-(4-fluorophenyl)-5-[2-(4-methoxyphenoxy)pyrimidin-4-yl]imidazole;

1-(piperidin-4-yl)-4-(4-fluorophenyl)-5-[2-(4-fluorophenoxy)pyrimidin-4-yl]imidazole;

1-(piperidin-4-yl)-4-(4-fluorophenyl)-5-[2-(4-aminocarbonylphenoxy)pyrimidin-4-yl]imidazole;

1-(piperidin-4-yl)-4-(4-fluorophenyl)-5-[2-(4-ethylphenoxy)pyrimidin-4-yl]imidazole;

1-(piperidin-4-yl)-4-(4-fluorophenyl)-5-[2-(4-benzyloxyphenoxy)pyrimidin-4-yl]imidazole;

1-(piperidin-4-yl)-4-(4-fluorophenyl)-5-[2-(4-cyanophenoxy)pyrimidin-4-yl]imidazole;

1-(piperidin-4-yl)-4-(4-fluorophenyl)-5-[2-(4-hydroxyphenoxy)pyrimidin-4-yl]imidazole;

1-(4-hydroxycyclohexyl)-4-(4-fluorophenyl)-5-[2-(phenoxy)pyrimidin-4-yl]imidazole;

1-(piperidin-4-yl)-4-(4-fluorophenyl)-5-[2-(2,6-dimethylphenoxy)pyridin-4-yl]imidazole;

1-(piperidin-4-yl)-4-(4-fluorophenyl)-5-[2-(4-methylphenoxy)pyridin-4-yl]imidazole;

1-(piperidin-4-yl)-4-(4-fluorophenyl)-5-[2-(4-chlorophenoxy)pyridin-4-yl]imidazole;

1-[3-(N-morpholino)propyl]-4-(4-fluorophenyl)-5-[2-(phenoxy)pyrimidin-4-yl]imidazole;

1-(piperidin-4-yl)-4-(4-fluorophenyl)-5-[2-(3-methoxyphenoxy)pyrimidin-4-yl]imidazole;

1-(piperidin-4-yl)-4-(4-fluorophenyl)-5-[2-(4-phenylphenoxy)pyrimidin-4-yl]imidazole;

1-(piperidin-4-yl)-4-(4-fluorophenyl)-5-[2-(4-phenoxyphenoxy)pyrimidin-4-yl]imidazole;

1-(piperidin-4-yl)-4-(4-fluorophenyl)-5-[2-(3-hydroxyphenoxy)pyrimidin-4-yl]imidazole;

1-(3-(N-morpholino)propyl)-4-(4-fluorophenyl)-5-[2-(4-fluorophenoxy)pyrimidin-4-yl]imidazole;

1-(piperidin-4-yl)-4-(4-fluorophenyl)-5-[2-(2-hydroxyphenoxy)pyrimidin-4-yl]imidazole;

1-(piperidin-4-yl)-4-(4-fluorophenyl)-5-[2-((3,4-methylenedioxy)phenoxy)pyrimidin-4-yl]imidazole;

1-(piperidin-4-yl)-4-(4-fluorophenyl)-5-[2-(3-fluorophenoxy)pyrimidin-4-yl]imidazole;

1-(piperidin-4-yl)-4-(4-fluorophenyl)-5-[2-(2-fluorophenoxy)pyrimidin-4-yl]imidazole;

1-(piperidin-4-yl)-4-(4-fluorophenyl)-5-[2-(2-methoxyphenoxy)pyrimidin-4-yl]imidazole;

1-(piperidin-4-yl)-4-(4-fluorophenyl)-5-[2-(3-trifluoromethylphenoxy)pyrimidin-4-yl]imidazole;

1-(piperidin-4-yl)-4-(4-fluorophenyl)-5-[2-(3,4-difluorophenoxy)pyrimidin-4-yl]imidazole;

1-(piperidin-4-yl)-4-(4-fluorophenyl)-5-[2-(4-methylsulfonylphenoxy)pyrimidin-4-yl]imidazole;

1-(4-piperidinyl)-4-(4-fluorophenyl)-5-(2-thiophenoxypyrimidin-4-yl)imidazole;

1-(4-piperidinyl)-4-(4-fluorophenyl)-5-[2-(1-methyltetrazol-5-ylthio)pyridin-4-yl]imidazole;

5-[2-(2-hydroxyethoxy)pyrimidin-4-yl]-4-(4-fluorophenyl)-1-(4-oxocyclohexyl)imidazole;

5-[2-(2-hydroxyethoxy)]pyrimidin-4-yl)-4-(4-fluorophenyl)-1-(4-hydroxycyclohexyl)imidazole;

5-[2-(2-tert-butylamino)ethoxypyrimidin-4-yl]-4-(4-fluorophenyl)-1-(4-oxocyclohexyl)imidazole;

5-[2-(2-tert-butylamino)ethoxypyrimidin-4-yl]-4-(4-fluorophenyl)-1-(4-hydroxycyclohexyl)imidazole;

1-(4-piperidinyl)-4-(4-Fluorophenyl)-5-(2-isopropoxy-4-pyrimidinyl)imidazole;

1-(4-piperidinyl)-4-(4-Fluorophenyl)-5-(2-methoxy-4-pyrimidinyl)imidazole;

5-(2-hydroxy-4-pyrimidinyl)-4-(4-fluorophenyl)-1-(4-piperidinyl)imidazole;

5-(2-methoxy-4-pyridinyl)-4-(4-fluorophenyl)-1-(4-piperidinyl)imidazole;

5-(2-isopropoxy-4-pyridinyl)-4-(4-fluorophenyl)-1-(4-piperidinyl)imidazole;

5-(2-methylthio-4-pyrimidinyl)-4-(4-fluorophenyl)-1-(4-piperidinyl)imidazole;

5-(2-methylthio-4-pyrimidinyl)-4-(4-fluorophenyl)-1-[1-methyl-4-piperidinyl)imidazole;

5-(2-ethoxy-4-pyrimidinyl)-4-(4-fluorophenyl)-1-(4-piperidinyl)imidazole;

1-(1-ethylcarboxylpiperidin-4-yl)-3-(4-thiomethylphenyl)-5-[2-(thiomethyl)pyrimidin-4-yl]-imidazole;

1-(1-ethylcarbonylpiperidin-4-yl)-4-(4-methylsulfinylphenyl)-5-[(2-methylsulfinyl)pyrimidin-4-yl]imidazole;

2-(4-methylthiophenyl)-4-(4-fluorophenyl)-5-(2-methoxy-4-pyrimidinyl)imidazole;

2-(4-methylsulfinylphenyl)-4-(4-fluorophenyl)-5-(2-methoxy-4-pyrimidinyl)imidazole;

2-[(4-N,N-dimethyl)aminomethylphenyl]-4-(4-fluorophenyl)-5-(2-methoxy-4-pyrimidinyl)imidazole;

2-[(4-N,N-dimethyl)aminomethylphenyl]-4-(4-fluorophenyl)-5-(2-phenoxy-4-pyrimidinyl)imidazole;

(+/−)-2-(4-methylsulfinylphenyl]-4-(4-fluorophenyl)-5-(2-phenoxy-4-pyrimidinyl)imidazole; and

2-(4-methylthiophenyl]-4-(4-fluorophenyl)-5-(2-phenoxy-4-pyrimidinyl)imidazole;

and pharmaceutically acceptable salts thereof.

Compounds useful in the practice of the present invention also include, but are not limited to, compounds of formula:

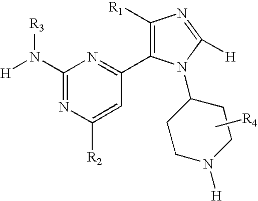

and pharmaceutically acceptable salts thereof,

wherein

R₁ is hydrogen, C₁₋₅ alkyl, halogen, C₁₋₅ alkoxy or arylC₁₋₅ alkyl;

R₂ and R₄ are independently hydrogen, C₁₋₅ alkyl, aryl, arylC₁₋₅ alkyl, heteroaryl, heteroarylC₁₋₅ alkyl, heterocyclic or heterocyclicC₁₋₅ alkyl; and

R₃ is hydrogen or C₁₋₃ alkyl.

Compounds useful in the practice of the present invention also include, but are not limited to, compounds of formula:

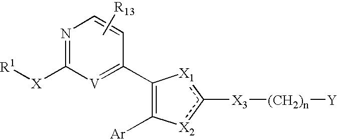

and pharmaceutically acceptable salts thereof,

wherein

X is O, CH₂, S or NH, or the moiety X—R₁ is hydrogen;

R¹ is hydrogen, C₁₋₆ alkyl, C₃₋₇ cycloalkyl, aryl, arylC₁₋₆ alkyl, heterocyclyl, heterocyclylC₁₋₆ alkyl, heteroaryl or heteroarylC₁₋₆ alkyl, each of which, except for hydrogen, is optionally substituted;

V is CH or N;

Ar is an aryl or heteroaryl ring, each of which is optionally substituted;

one of X₁ and X₂ is N, and the other is NR¹⁵, wherein R¹⁵ is hydrogen, C₁₋₆ alkyl or arylC₁₋₆ alkyl;

X₃ is a covalent bond or C(R₂)(R₃);

R² and R³ independently represent optionally substituted C₁₋₆ alkyl, or R² and R³ together with the carbon atom to which they are attached form an optionally substituted C₃₋₇ cycloalkyl, C₃₋₇ cycloalkenyl or 5- to 7-membered heterocyclyl ring containing up to three heteroatoms independently selected from the group consisting of N, O and S;

n is 0, 1, 2, 3 or 4;

Y is NR¹⁰R¹¹, NR¹⁰C(Z)NR¹⁰R¹¹, NR¹⁰COOR¹¹, NR¹⁰SO₂R¹¹or C(O)NR⁴R⁵;

R⁴ and R⁵ independently represent hydrogen, C₁₋₆ alkyl, C₃₋₇ cycloalkyl, aryl, arylC₁₋₆ alkyl, heteroaryl, heteroarylC₁₋₆ alkyl, heterocyclyl or heterocyclylC₁₋₆ alkyl, each of which, except hydrogen, is optionally substituted, or R⁴ and R⁵ together with the nitrogen atom to which they are attached form a 4- to 10-membered optionally-substituted monocyclic or bicyclic ring;

R¹³ is hydrogen, X—R₁, halogen, optionally-substituted C₁₋₆ alkylsulfinyl, CH₂OR¹⁴, di-C₁₋₆ alkylamino, N(R⁶)C(O)R⁷, N(R⁶)S(O)₂R⁸ or a 5- to 7-membered N-heterocyclyl ring which optionally contains an additional heteroatom selected from the group consisting of O, S and NR⁹;

R¹⁴ is hydrogen, —C(Z)R¹², optionally-substituted C₁₋₆ alkyl, optionally-substituted aryl, optionally-substituted arylC₁₋₆ alkyl or S(O)₂R⁸;

R is hydrogen or C₁₋₆ alkyl;

R⁷ is hydrogen, C₁₋₆ alkyl, C₃₋₇ cycloalkyl, aryl, arylC₁₋₆ alkyl, heteroaryl, heteroarylC₁₋₆ alkyl, heterocyclyl or heterocyclylC₁₋₆ alkyl;

R⁸ is C₁₋₆ alkyl, C₃₋₇ cycloalkyl, aryl, arylC₁₋₆ alkyl, heteroaryl, heteroarylC₁₋₆ alkyl, heterocyclyl or heterocyclylC₁₋₆ alkyl;

R⁹ is hydrogen, cyano, C₁₋₄ alkyl, C₃₋₇ cycloalkyl or aryl;

R¹⁰, R¹¹ and R¹² are independently selected from the group consisting of hydrogen, C₁₋₆ alkyl, C₃₋₇ cycloalkyl, heterocyclyl, heterocyclylC₁₋₆ alkyl, heterocyclylC₂₋₆ alkenyl, aryl, arylC₁₋₆ alkyl, arylC₂₋₆ alkenyl, heteroaryl, heteroarylC₁₋₆ alkyl and heteroarylC₂₋₆ alkenyl, each of which is optionally substituted; or NR¹⁰R¹¹ can represent a 5- to 7-membered heterocyclyl ring optionally containing an additional heteroatom selected from the group consisting of O, N and S; and

Z is oxygen or sulfur.

Compounds useful in the practice of the present invention also include, but are not limited to, compounds of formulas:

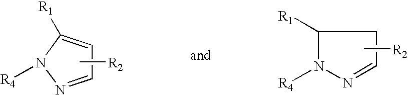

and pharmaceutically acceptable salts thereof,

wherein

R₁ is a heteroaryl ring selected from the group consisting of 4-pyridyl, 4-pyrimidinyl, 4-quinolyl, 6-isoquinolinyl, quinazolin-4-yl, 1-imidazolyl, 1-benzimidazolyl, 4-pyridazinyl and 1,2,4-triazin-5-yl, which heteroaryl ring is substituted one to three times with Y, NHRₐ, optionally-substituted C₁₋₄ alkyl, halogen, hydroxyl, optionally-substituted C₁₋₄ alkoxy, optionally-substituted C₁₋₄ alkylthio, optionally-substituted C₁₋₄ alkylsulfinyl, CH₂OR₁₂, amino, mono- or di-C₁₋₆ alkyl-substituted amino, N(R₁₀)C(O)R_{b}, N(R₁₀)S(O)₂R_{d} or an N-heterocyclyl ring which has from 5 to 7 members and optionally contains an additional heteroatom selected from the group consisting of oxygen, sulfur and NR₁₅;

Y is X₁—Rₐ;

X₁ is oxygen or sulfur;

Rₐ is C₁₋₆ alkyl, aryl, arylC₁₋₆ alkyl, heterocyclic, heterocyclylC₁₋₆ alkyl, heteroaryl or heteroarylC₁₋₆ alkyl, wherein each of these moieties is optionally substituted;

R_{b} is hydrogen, C₁₋₆ alkyl, C₃₋₇ cycloalkyl, aryl, arylC₁₋₄ alkyl, heteroaryl, heteroarylC₁₋₄ alkyl, heterocyclyl or heterocyclylC₁₋₄ alkyl;

R_{d} is C₁₋₆ alkyl, C₃₋₇ cycloalkyl, aryl, arylC₁₋₄ alkyl, heteroaryl, heteroarylC₁₋₄ alkyl, heterocyclyl or heterocyclylC₁₋₄ alkyl;

R₄ is phenyl, naphth-1-yl, naphth-2-yl, a heteroaryl or a fused phenyl-containing ring system, which is optionally substituted by one or two substituents, each of which is independently selected, and which, for a 4-phenyl, 4-naphth-1-yl, 5-naphth-2-yl or 6-naphth-2-yl substituent, is halogen, cyano, nitro, —C(Z)NR₇R₁₇, —C(Z)OR₁₆, —(CR₁₀R₂₀)ᵥCOR₁₂, —SR₅, —SOR₅, —OR₁₂, halo-substituted-C₁₋₄ alkyl, C₁₋₄ alkyl, -ZC(Z)R₁₂, —NR₁OC(Z)R₁₆ or —(CR₁OR₂₀)ᵥNR₁₀R₂₀, and which, for other positions of substitution, is halogen, cyano, nitro, phenyl, —C(Z)NR₁₃R₁₄, —C(Z)OR_{f}, —(CR₁₀R₂₀)ₘ″COR_{f}, —S(O)ₘR_{f}, —OR_{f}, halo-substituted C₁₋₄ alkyl, C₁₋₁₀ alkyl, -ZC(Z)R_{f}, optionally-substituted phenyl, —(CR₁₀R₂₀)_{m″}NR₁₀C(Z)R_{f}, —NR₁₀S(O)_{m″}R₈, —NR₁₀S(O)_{m″}NR₇R₁₇, -ZC(Z)R₁₂ or —(CR₁₀R₂₀)ₘ″NR₁₃R₁₄;

R_{f} is heterocyclyl, heterocyclylC₁₋₁₀alkyl or R₈;

v is 0, 1 or 2;

m is 0, 1 or 2;

m′ is 1 or 2;

m″ is 0, 1, 2, 3, 4 or 5;

R₂ hydrogen, —(CR₁₀R₂₃)ₙOR₉, heterocylyl, heterocyclylC₁₋₁₀alkyl, C₁₋₁₀alkyl, halo-substituted C₁₋₁₀alkyl, C₂₋₁₀ alkenyl, C₂₋₁₀ alkynyl, C₃₋₇ cycloalkyl, C₃₋₇ cycloalkylC₁₋₁₀alkyl, C₅₋₇ cycloalkenyl, C₅₋₇cycloalkenylC₁₋₁₀alkyl, aryl, arylC₁₋₁₀ alkyl, heteroaryl, heteroarylC l-o alkyl, (CR₁₀R₂₃)ₙOR₁₁, (CR₁₀R₂₃)ₙS(O)ₘR₁₈, (CR₁₀R₂₃)ₙNHS(O)₂R₁₈, (CR₁₀R₂₃)ₙNR₁₃R₁₄, (CR₁₀R₂₃)ₙNO₂, (CR₁OR₂₃)ₙCN, (CR₁OR₂₃)ₙS(O)ₙNR₁₃R₁₄, (CR₁OR₂₃)ₙC(Z)R₁₁, (CR₁OR₂₃)ₙOC(Z)R₁₁, (CR₁₀R₂₃)ₙC(Z)OR₁₁, (CR₁₀R₂₃)ₙC(Z)NR₁₃R₁₄, (CR₁₀R₂₃)ₙC(Z)NR₁₀R₉, (CR₁₀R₂₃)ₙNR₁OC(Z)R₁₁, (CR₁₀R₂₃)ₙNR₁OC(Z)NR₁₃R₁₄, (CR₁₀R₂₃)ₙN(OR₆)C(Z)NR₁₃R₁₄, (CR₁₀R₂₃)ₙN(OR₆)C(Z)R₁, (CR₁₀R₂₃)ₙC(═NOR₆)R₁, (CR₁₀R₂₃)ₙNR₁₀C(═NR₁₉)NR₁₃R₁₄, (CR₁₀R₂₃)ₙOC(Z)NR₁₃R₁₄, (CR₁₀R₂₃)ₙNR₁OC(Z)NR₁₃R₁₄, (CR₁₀R₂₃)ₙNR₁₀C(Z)OR₁₀, 5-(R₁₈)-1,2,4-oxadiazol-3-yl or 4-(R₁₂)-5-(R₁₈R₁₉)-4,5-dihydro-1,2,4-oxadiazol-3-yl; wherein the aryl, arylalkyl, heteroaryl, heteroaryl alkyl, cycloalkyl, cycloalkyl alkyl, heterocyclic and heterocyclic alkyl groups are optionally substituted;

n is 0, or an integer having a value of 1 to 10;

Z is oxygen or sulfur;

R₅ is hydrogen, C₁₋₄ alkyl, C₂₋₄ alkenyl, C₂₋₄ alkynyl or NR₇R₁₇, excluding the moieties —SR₅ being —SNR₇R₁₇ and —S(O)R₅ being —SOH;

R₆ is hydrogen, a pharmaceutically-acceptable cation, C₁₋₁₀ alkyl, C₃₋₇ cycloalkyl, aryl, arylC₁₋₄ alkyl, heteroaryl, heteroarylC₁₋₄ alkyl, heterocyclyl, aroyl or C₁₋₁₀alkanoyl;

R₇ and R₁₇ are independently selected from the group consisting of hydrogen and C₁₋₄ alkyl, or R₇ and R₁₇ together with the nitrogen to which they are attached form a heterocyclic ring of 5 to 7 members which ring optionally contains an additional heteroatom selected from the group consisting of oxygen, sulfur and NR₁₅; R₈ is C₁₋₁₀ alkyl, halo-substituted C₁₋₁₀alkyl, C₂₋₁₀ alkenyl, C₂₋₁₀alkynyl, C₃₋₇ cycloalkyl, C₅₋₇ cycloalkenyl, aryl, arylC₁₋₁₀alkyl, heteroaryl, heteroarylC₁₋₁₀alkyl, (CR₁₀R₂₀)ₙOR₁₁, (CR₁₀R₂₀)ₙS(O)_{m″}R₈, (CR₁₀R₂₀)ₙNHS(O)₂R₁₈ or (CR₁₀R₂₀)ₙNR₁₃R₁₄, wherein the aryl, arylalkyl, heteroaryl and heteroaryl alkyl are optionally substituted;

R₉ is hydrogen, —C(Z)R₁₁, optionally-substituted C₁₋₁₀ alkyl, S(O)₂R₁₈, optionally-substituted aryl or optionally-substituted arylC₁₋₄ alkyl;

R₁₀ and R₂₀ are independently selected from the group consisting of hydrogen and C₁₋₄ alkyl;

R₁ is hydrogen, C₁₋₁₀ alkyl, C₃₋₇ cycloalkyl, heterocyclyl, heterocyclylC₁₋₁₀ alkyl, aryl, arylC₁₋₁₀ alkyl, heteroaryl or heteroarylC₁₋₁₀ alkyl, wherein the aryl, arylalkyl, heteroaryl, heteroaryl alkyl, heterocyclyl and heterocyclylalkyl are optionally substituted;

R₁₂ is hydrogen or R₁₆;

R₁₃ and R₁₄ are independently selected from the group consisting of hydrogen, optionally-substituted C₁₋₄ alkyl, optionally-substituted aryl and optionally-substituted arylC₁₋₄ alkyl, or together with the nitrogen to which they are attached form a heterocyclic ring of 5 to 7 members which ring optionally contains an additional heteroatom selected from the group consisting of oxygen, sulfur and NR₉;

R₁₅ is hydrogen, C₁₋₄ alkyl or C(Z)-C₁₋₄ alkyl;

R₁₆ is C₁₋₄ alkyl, halo-substituted C₁₋₄ alkyl or C₃₋₇ cycloalkyl;

R₁₈ is C₁₋₁₀ alkyl, C₃₋₇ cycloalkyl, heterocyclyl, aryl, arylC₁₋₁₀ alkyl, heterocyclyl, heterocyclylC₁₋₁₀alkyl, heteroaryl or heteroarylC₁₋₁₀alkyl, wherein the aryl, arylalkyl, heteroaryl, heteroaryl alkyl, heterocyclyl and heterocyclylalkyl are optionally substituted;

R₁₉ is hydrogen, cyano, C₁₋₄ alkyl, C₃₋₇ cycloalkyl or aryl; and

R₂₃ is hydrogen, C₁₋₆ alkyl, C₃₋₇ cycloalkyl, aryl, arylC₁₋₄ alkyl, heteroaryl, heteroarylC₁₋₄ alkyl, heterocyclyl or heterocyclylC₁₋₄ alkyl, all of which are optionally substituted.

Exemplary compounds of these formulas include:

4-[1-(4-fluorophenyl)-3-phenyl-1H-pyrazol-5-yl]pyridine;

4-[4-bromo-1-(4-fluorophenyl)-3-phenyl-1H-pyrazol-5-yl]pyridine;

4-[1-(4-fluorophenyl)-3-[4-(methylthio)phenyl]-1H-pyrazol-5-yl]pyridine;

4-[1-(4-fluorophenyl)-3-[4-(methylsulfonyl)phenyl]-1H-pyrazol-5-yl]pyridine;

4-[1-(4-fluorophenyl)-3-[4-(methylsulfinyl)phenyl]-1H-pyrazol-5-yl]pyridine;

4-[1-(4-fluorophenyl)-4,5-dihydro-3-phenyl-1H-pyrazol-5-yl]pyridine; and

4-[1-(4-fluorophenyl)-4,5-dihydro-3-[4-(methylthio)phenyl]-1H-pyrazol-5-yl]pyridine;

and pharmaceutically acceptable salts thereof.

Compounds useful in the practice of the present invention also include, but are not limited to, compounds of formulas:

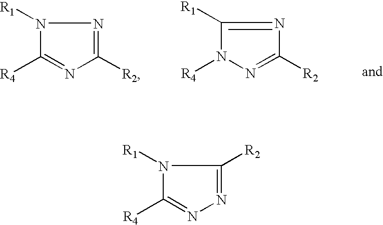

and pharmaceutically acceptable salts thereof,

wherein

R₁ is 4-pyridyl or 4-pyrimidinyl ring, which ring is optionally substituted one or more times with Y, C₁₋₄ alkyl, halogen, hydroxyl, C₁₋₄ alkoxy, C₁₋₄ alkylthio, C₁₋₄ alkylsulfinyl, CH₂OR₁₂, amino, mono- or di-C₁₋₆ alkyl-substituted amino, N(R₁₀)C(O)R_{b}or an N-heterocyclyl ring which has from 5 to 7 members and optionally contains an additional heteroatom selected from the group consisting of oxygen, sulfur and NR₁₅;

Y is X₁—Rₐ;

X₁ is oxygen, sulfur or NH;

Rₐ is C₁₋₆ alkyl, aryl, arylC₁₋₆ alkyl, heterocyclic, heterocyclylC₁₋₆ alkyl, heteroaryl or heteroarylC₁₋₆ alkyl, wherein each of these moieties is optionally substituted;

R_{b} is hydrogen, C₁₋₆ alkyl, C₃₋₇ cycloalkyl, aryl, arylC₁₋₄ alkyl, heteroaryl, heteroarylC₁₋₄ alkyl, heterocyclyl or heterocyclylC₁₋₄ alkyl, wherein each of these moieties is optionally substituted;

R₄ is phenyl, naphth-1-yl, naphth-2-yl or a heteroaryl, which is optionally substituted by one or two substituents, each of which is independently selected, and which, for a 4-phenyl, 4-naphth-1-yl, 5-naphth-2-yl or 6-naphth-2-yl substituent, is halogen, cyano, nitro, —C(Z)NR₇R₁₇, —C(Z)OR₁₆, —(CR₁₀R₂₀)ₙCOR₁₂, —SR₅, —SOR₅, —OR₁₂, halo-substituted-C₁₋₄ alkyl, C₁₋₄ alkyl, -ZC(Z)R₁₂, —NR₁OC(Z)R₁₆ or —(CR₁₀R₂₀)_{m″}NR₁₀R₂₀ and which, for other positions of substitution, is halogen, cyano, —C(Z)NR₁₃R₁₄, —C(Z)OR_{f}, —(CR₁₀R₂₀)ₘ″COR_{f}, —S(O)ₘR_{f}, —OR_{f}, halo-substituted C₁₋₄ alkyl, C₁₋₄ alkyl, -ZC(Z)R_{f}, —(CR₁₀R₂₀)ₘ′NR₁₀C(Z)R_{f}, —NR₁₀S(O)_{m′}R₈, —NR₁₀S(O)_{m′}NR₇R₁₇ or —(CR₁₀R₂₀)ₘ″NR₁₃R₁₄;

R_{f} is heterocyclyl, heterocyclylC₁₋₁₀alkyl or R₈;

v is 0, 1 or 2;

m is 0, 1 or 2;

m′ is 1 or 2;

m″ is 0, 1, 2, 3, 4 or 5;

R₂ hydrogen, C(H)(A)(R₂₂), —(CR₁₀R₂₃)ₙOR₉, heterocylyl, heterocyclylC₁₋₁₀ alkyl, C₁₋₁₀alkyl, halo-substituted C₁₋₁₀alkyl, C₂₋₁₀ alkenyl, C₂-10 alkynyl, C₃₋₇ cycloalkyl, C₃₋₇ cycloalkylC₁₋₁₀alkyl, C₅₋₇ cycloalkenyl, C₅₋₇cycloalkenylC₁₋₁₀alkyl, aryl, arylC₁₋₁₀alkyl, heteroaryl, heteroarylC₁₋₁₀alkyl, (CR₁₀R₂₃)ₙOR₁₁, (CR₁₀R₂₃)ₙS(O)ₘR₁₈, (CR₁₀R₂₃)ₙNHS(O)₂R₁₈, (CR₁₀R₂₃)ₙNR₁₃R₁₄, (CR₁OR₂₃)ₙNO₂, (CR₁₀R₂₃)ₙCN, (CR₁₀OR₂₃)ₙS(O)ₘNR₁₃R₁₄, (CR₁₀R₂₃)ₙC(Z)R₁₁, (CR₁OR₂₃)ₙOC(Z)R₁₁, (CR₁₀R₂₃)ₙC(Z)OR₁₁, (CR₁₀R₂₃)ₙC(Z)NR₁₃R₁₄, (CR₁₀R₂₃)ₙC(Z)NR₁₀R₉, (CR₁₀R₂₃)ₙNR₁OC(Z)R₁₁, (CR₁₀R₂₃)ₙNR₁₀C(Z)NR₁₃R₁₄, (CR₁₀R₂₃)ₙN(OR₆)C(Z)NR₁₃R₁₄, (CR₁₀R₂₃)ₙN(OR₆)C(Z)R₁, (CR₁₀R₂₃)ₙC(═NOR₆)R₁₁, (CR₁₀R₂₃)ₙNR₁OC(═NR₁₉)NR₁₃R₁₄, (CR₁₀R₂₃)ₙOC(Z)NR₁₃R₁₄, (CR₁₀R₂₃)ₙNR₁OC(Z)NR₁₃R₁₄, (CR₁₀R₂₃)ₙNR₁OC(Z)OR₁₀, 5-(R₁₈)-1,2,4-oxadiazol-3-yl or 4-(R₁₂)-5-(R₁₈R₁₉)-4,5-dihydro-1,2,4-oxadiazol-3-yl; wherein the aryl, arylalkyl, heteroaryl, heteroaryl alkyl, cycloalkyl, cycloalkyl alkyl, heterocyclic and heterocyclic alkyl groups are optionally substituted;

A is an optionally-substituted aryl, heterocyclyl or heteroaryl ring, or A is a substituted C₁₋₁₀ alkyl;

n is 0, or an integer having a value of 1 to 10;

Z is oxygen or sulfur;

R₅ is hydrogen, C₁₋₄ alkyl, C₂₋₄ alkenyl, C₂₋₄ alkynyl or NR₇R₁₇, excluding the moieties —SR₅ being —SNR₇R₁₇ and —S(O)R₅ being —SOH;

R₆ is hydrogen, a pharmaceutically-acceptable cation, C₁₋₁₀ alkyl, C₃₋₇ cycloalkyl, aryl, arylC₁₋₄ alkyl, heteroaryl, heteroarylC₁₋₄ alkyl, heterocyclyl, aroyl or C₁₋₁₀alkanoyl;

R₇ and R₁₇ are independently selected from the group consisting of hydrogen and C₁₋₄ alkyl, or R₇ and R₁₇ together with the nitrogen to which they are attached form a heterocyclic ring of 5 to 7 members which ring optionally contains an additional heteroatom selected from the group consisting of oxygen, sulfur and NR₁₅;

R₈ is C₁₋₁₀ alkyl, halo-substituted C₁₋₁₀ alkyl, C₂₋₁₀ alkenyl, C₂₋₁₀ alkynyl, C₃₋₇ cycloalkyl, C₅₋₇ cycloalkenyl, aryl, arylC₁₋₁₀alkyl, heteroaryl, heteroarylC₁₋₁₀alkyl, (CR₁₀R₂₀)ₙOR₁₁, (CR₁₀R₂₀)ₙS(O)ₘR₁₈, (CR₁₀R₂₀)ₙNHS(O)₂R₁₈ or (CR₁₀R₂₀)ₙNR₁₃R₁₄, wherein the aryl, arylalkyl, heteroaryl and heteroaryl alkyl are optionally substituted;

R₉ is hydrogen, —C(Z)R₁₁, optionally-substituted C₁₋₁₀ alkyl, S(O)₂R₁₈, optionally-substituted aryl or optionally-substituted arylC₁₋₄ alkyl;

R₁₀ and R₂₀ are independently selected from the group consisting of hydrogen and C₁₋₄ alkyl;

R₁₁ is hydrogen, C₁₋₁₀alkyl, C₃₋₇ cycloalkyl, heterocyclyl, heterocyclylC₁₋₁₀alkyl, aryl, arylC₁₋₁₀alkyl, heteroaryl or heteroarylC₁₋₁₀alkyl, wherein the aryl, arylalkyl, heteroaryl, heteroaryl alkyl, heterocyclyl and heterocyclylalkyl are optionally substituted;

R₁₂ is hydrogen or R₁₆;

R₁₃ and R₁₄ are independently selected from the group consisting of hydrogen, optionally-substituted C₁₋₄ alkyl, optionally-substituted aryl and optionally-substituted arylC₁₋₄ alkyl, or together with the nitrogen to which they are attached form a heterocyclic ring of 5 to 7 members which ring optionally contains an additional heteroatom selected from the group consisting of oxygen, sulfur and NR₉;

R₁₅ is R₁₀ or C(Z)C₁₋₄ alkyl;

R₁₆ is C₁₋₄ alkyl, halo-substituted C₁₋₄ alkyl or C₃₋₇ cycloalkyl;

R₁₈ is C₁₋₁₀alkyl, C₃₋₇ cycloalkyl, heterocyclyl, aryl, arylC₁₋₁₀ alkyl, heterocyclyl, heterocyclylC₁₋₁₀ alkyl, heteroaryl or heteroarylC₁₋₁₀ alkyl;

R₁₉ is hydrogen, cyano, C₁₋₄ alkyl, C₃₋₇ cycloalkyl or aryl; and

R₂₃ is hydrogen, C₁₋₆ alkyl, C₃₋₇ cycloalkyl, aryl, arylC₁₋₄ alkyl, heteroaryl, heteroarylC₁₋₄ alkyl, heterocyclyl or heterocyclylC₁₋₄ alkyl, all of which are optionally substituted.

Exemplary compounds of these formulas include:

1-(pyrid-4-yl)-3-phenyl-5-(4-fluorophenyl)-1,2,4-triazole;

1-(6-aminopyrimidin-4-yl)-3-phenyl-5-(4-fluorophenyl)-1,2,4-triazole;

1-[4-(6,7-dimethoxyquinazoline)]-3-phenyl-5-(4-fluorophenyl)-1,2,4-triazole;

1-(4-fluorophenyl)-3-phenyl-5-(2-aminopyrimidin-4-yl)-1,2,4-triazole; and

3-(4-fluorophenyl)-4-(2-aminopyrimidin-4-yl)-5-phenyl-1,2,4-triazole;

and pharmaceutically acceptable salts thereof.

Compounds useful in the practice of the present invention also include, but are not limited to, compounds of formula:

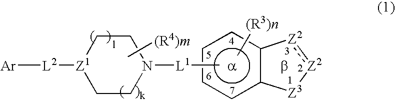

and pharmaceutically acceptable salts thereof,

wherein

[symbol] represents a single or double bond;

one Z² is CA or C(R⁸)A and the other is CR¹, CR¹₂, NR⁶ or N, wherein each of R¹, R⁶ and R⁸ is independently hydrogen or a noninterfering substituent;

A is —Wᵢ—C(O)XⱼY wherein Y is —C(O)R² or an isostere thereof and R² is hydrogen or a noninterfering substituent, each of W and X is a spacer preferably 2-6A, and each of i and j is independently 0 or 1;

Z³ is NR⁷ or 0, wherein R⁷ is optionally substituted alkyl, alkenyl, alkynyl, aryl, arylalkyl, acyl, aroyl, heteroaryl, heteroalkyl, heteroalkenyl, heteroalkynyl or heteroalkylaryl; or R⁷ is H, —S(O)R, —S(O)₂R, —C(O)R, —C(O)OR, alkylene-C(O)R, —S(O)₂OR, —C(O)NR₂, —S(O)₂NR₂, —CN, —CF₃, —NR₂, —OR, alkylene-SR, alkylene-S(O)R, alkylene-S(O)₂OR, alkylene-OC(O)R, alkylene-C(O)OR, alkylene-CN, alkylene-C(O)NR₂ or —SiR₃, wherein each R is independently H, alkyl, alkenyl or aryl, or heteroatom-containing forms thereof.

each R³ is independently a noninterfering substituent;

n is 0-3;

each of L¹ and L² is independently a linker;

each R⁴ is independently a noninterfering substituent;

m is 0-4;

Z¹ is CR⁵ or N, wherein R⁵ is hydrogen or a noninterfering substituent;

each of l and k is independently an integer from 0-2, wherein the sum of l and k is 0-3;

Ar is an aryl group substituted with 0-5 noninterfering substituents, wherein two noninterfering substituents can form a fused ring; and

the distance between the atom of Ar linked to L² and the center of the α-ring is preferably 4.5-24 Å.

The following definitions apply to compounds of formula (1) that are useful in the practice of the present invention:

As used herein, a “noninterfering substituent” is a substituent which leaves the ability of the compound of formula (1) to inhibit p38-α activity qualitatively intact. Thus, the substituent may alter the degree of inhibition of p38-α. However, as long as the compound of formula (1) retains the ability to inhibit p38-α activity, the substituent will be classified as “noninterfering.” A number of assays for determining the ability of any compound to inhibit p38-α activity are available in the art. A whole blood assay for this evaluation is illustrated below: the gene for p38-α has been cloned and the protein can be prepared recombinantly and its activity assessed, including an assessment of the ability of an arbitrarily chosen compound to interfere with this activity. The essential features of the molecule are tightly defined. The positions which are occupied by “noninterfering substituents” can be substituted by conventional organic moieties as is understood in the art. It is irrelevant to the present invention to test the outer limits of such substitutions. The essential features of the compounds are those set forth with particularity herein.

The term “hydrocarbyl residue” means a residue that contains only carbon and hydrogen. The residue can be saturated or unsaturated, aromatic or nonaromatic, straight-chained or branched, cyclic or acyclic. When so stated, however, the hydrocarbyl residue can contain heteroatoms over and above the carbon and hydrogen members of the substituent residue. Thus, when specifically noted as containing heteroatoms, the hydrocarbyl residue can also contain, e.g., carbonyl groups, amino groups, hydroxyl groups, and the like, or contain heteroatoms within the “backbone” of the residue.

The term “inorganic residue” means a residue that does not contain carbon. Examples include halo, hydroxyl, —NO₂ and —NH₂.

The terms “alkyl,” “alkenyl” and “alkynyl” include straight- and branched-chain and cyclic monovalent substituents. Examples include methyl, ethyl, isobutyl, cyclohexyl, cyclopentylethyl, 2-propenyl, 3-butynyl and the like. The alkyl substituents contain typically 1-10, preferably 1-6, carbon atoms. The alkenyl and alkynyl substituents contain typically 2-10, preferably 2-6, carbon atoms. The terms “heteroalkyl,” “heteroalkenyl” and “heteroalkynyl” are similarly defined, but contain within the backbone residue 1 or 2 heteroatoms independently selected from the group consisting of O, S and N.

The term “acyl” encompasses the definitions of alkyl, alkenyl and alkynyl, and their related heteroatom-containing forms, that are coupled to an additional residue through a carbonyl group.

An “aromatic” moiety means a monocyclic or fused bicyclic moiety. Examples include phenyl and naphthyl. The term “heteroaromatic” refers to monocyclic or fused bicyclic ring systems containing one or more heteroatoms selected from the group consisting of O, S and N. Examples include pyridyl, pyrimidyl, indolyl, benzimidazolyl, benzotriazolyl, isoquinolyl, quinolyl, benzothiazolyl, benzofuranyl, thienyl, furyl, pyrrolyl, thiazolyl, oxazolyl and imidazolyl. Any monocyclic or fused bicyclic system that has the characteristics of aromaticity in terms of electron distribution throughout the ring system is included in one of these definitions. Typically, the ring systems contain 5-12 member atoms.

The terms “arylalkyl” and “heteroalkyl” refer to aromatic and heteroaromatic systems, respectively, that are coupled to another residue through a carbon chain. Said carbon chain is typically of 1-6 carbon atoms, and may be substituted or unsubstituted, saturated or unsaturated. Said carbon chains may also include a carbonyl group, thus making them able to provide substituents as an acyl moiety.

When the compounds of formula (1) are chiral, the invention includes the use of optically pure forms as well as mixtures of stereoisomers (including enantiomers).

In one embodiment, [symbol] represents a double bond.

In one embodiment, said CA or CR⁸A is in the 3-position. In one embodiment, said CA or CR⁸A is CA.

When R⁸ is present, examples of useful R⁸ include H, halo, alkyl, alkenyl and the like. Further examples of useful R⁸ include relatively small substituents corresponding, e.g., to H or C₁₋₄ alkyl.

In one embodiment, said CR¹, CR¹², NR⁶ or N is CR¹.

Examples of useful R¹ include hydrogen, alkyl, alkenyl, alkynyl, aryl, arylalkyl, acyl, aroyl, heteroaryl, heteroalkyl, heteroalkenyl, heteroalkynyl, heteroalkylaryl, —NH-aroyl, halo, —OR, —NR₂, —SR, —S(O)R, —S(O)₂R, —OC(O)R, —N(R)C(O)R, —N(R)C(O)NR₂, —N(R)C(O)OR, —OC(O)NR₂, —C(O)R, —C(O)OR, alkylene-C(O)OR, —S(O)₂₀R, —C(O)NR₂, —S(O)₂NR₂, —N(R)S(O)₂NR₂, —CN, —CF₃, —SiR₃ and —NO₂, wherein each R is independently H, alkyl, alkenyl or aryl, or heteroatom-containing forms thereof, and wherein two of R¹ can be joined to form a fused, 3- to 8-membered optionally substituted, aromatic or non-aromatic, saturated or unsaturated ring. Further examples of useful R¹ include hydrogen, alkyl, acyl, aryl, arylalkyl, heteroalkyl, heteroaryl, halo, —OR, —NR₂, —SR, —N(R)C(O)R, alkylene-C(O)OR, —C(O)R, —C(O)OR and —CN, wherein each R is independently H, alkyl or aryl, or heteroatom-containing forms thereof. In one embodiment, R¹ is H or alkyl, e.g., methyl.

Examples of useful R⁶ (when Z² is NR⁶) include hydrogen, alkyl, alkenyl, alkynyl, aryl, arylalkyl, acyl, aroyl, heteroaryl, heteroalkyl, heteroalkenyl, heteroalkynyl, heteroalkylaryl, —S(O)R, —S(O)₂R, —C(O)R, —C(O)OR, alkylene-C(O)R, —S(O)₂OR, —C(O)NR₂, —S(O)₂NR₂, —CN, —CF₃ and —SiR₃, wherein each R is independently H, alkyl, alkenyl or aryl, or heteroatom-containing forms thereof.

A “spacer” is any multivalent radical that is consistent with the 2-6 Å preferred distance requirement. The identities of spacers W and X are less important than the distances they impart between portions of the molecule. Examples of useful spacers include optionally substituted alkyl, alkenyl and alkynyl. Preferably, W and X are unsubstituted.

In one embodiment, i is zero. In one embodiment, j is zero.

In one embodiment, the α/β ring system is an indole having CA in the 3-position wherein A is —C(O)C(O)R².

The noninterfering substituent represented by R² (when R² is not H) is a C₁₋₂₀ hydrocarbyl residue containing 0-5 heteroatoms selected from O, S and N; or R² is an inorganic residue. In one embodiment, R² is H; or R² is straight- or branched-chain alkyl, alkenyl, alkynyl, aryl, arylalkyl, heteroalkyl, heteroaryl, or heteroarylalkyl, each of which is optionally substituted with halo, alkyl, heteroalkyl, —SR, —OR, —NR₂, —OC(O)R, —N(R)C(O)R, —N(R)C(O)NR₂, —N(R)S(O)₂R, —N(R)S(O)₂NR₂, —OC(O)NR₂, —CN, —C(O)OR, —C(O)NR₂, —C(O)R or —SiR₃, wherein each R is independently H, alkyl, alkenyl or aryl, or the heteroatom-containing forms thereof; or R² is —OR, —NR₂, —SR, —N(R)C(O)NR₂, —OC(O)NR₂, or —N(R)S(O)₂NR₂, wherein each R is independently H, alkyl, alkenyl or aryl, or the heteroatom-containing forms thereof, and wherein two R attached to the same atom together with said atom optionally form a 3- to 8-membered ring, wherein said ring is optionally further substituted by alkyl, alkenyl, alkynyl, aryl, arylalkyl, heteroalkyl, heteroaryl or heteroarylalkyl, each of which is optionally substituted by halo, —SR, —OR, —NR₂, —OC(O)R, —N(R)C(O)R, —N(R)C(O)NR₂, —N(R)S(O)₂R, —N(R)S(O)₂NR₂, —OC(O)NR₂ or —SiR₃, wherein each R is independently H, alkyl, alkenyl or aryl, or heteroatom-containing forms thereof, and wherein two R attached to the same atom together with said atom optionally form a 3- to 8-membered ring, wherein said ring is optionally substituted as defined above. Examples of useful R² also include H, heteroarylalkyl, —NR₂, heteroaryl, —C(O)OR, —N(R)NR₂, heteroarylene-C(O)OR, heteroaryloxy, —OR, heteroarylene-NR₂, —N(R)OR and alkyl. Further examples of useful R² include isopropylpiperazinyl, methylpiperazinyl, dimethylamino, piperazinyl, isobutyloxycarbonyl, ethylcarbonyloxy, morpholinyl, dimethylaminoethylamino, isobutyloxycarbonyl-piperazinyl, piperazinyloxy, ethoxycarbonylpiperazinyl, methoxy, ethoxy, hydroxy, methyl, amino, pyrrolidinylethylamino, dihydroxypropylamino, piperidinyl, pyrrolidinylpiperidinyl, and methylpiperidinyl. Further examples of useful R² also include methoxy, amino, dimethylamino, methylpiperazinyl, tert-butoxycarbonylpiperazinyl and morpholinyl.

Isosteres of —C(O)R₂ as represented by Y include tetrazolyl; 1,2,3-triazolyl optionally substituted at the unattached carbon in the ring by —SCH₃, —C(O)CH₃, —Br, —S(O)CH₃, —S(O)₂CH₃, —NO₂, —CF₃, —CN or —C(O)OCH₃; 1,2,4-triazolyl optionally substituted at the unattached carbon in the ring by —SCH₃, —C(O)CH₃, —Br, —S(O)CH₃, —S(O)₂CH₃ or —NO₂; and imidazolyl optionally substituted at an unattached carbon in the ring by —SCH₃, —C(O)CH₃, Br, —S(O)CH₃, —S(O)₂CH₃ or —NO₂.

Examples of useful R⁷ (when Z³ is NR⁷) include H, C₁₋₄ alkyl, C₁₋₄ acyl, and —C(O)OR wherein R is H, alkyl, alkenyl or aryl, or heteroatom-containing forms thereof. When R⁷ is C₁₋₄ alkyl it is preferably methyl. Preferred R⁷ also include substituted alkyl wherein the preferred substituents form ether linkages or contain sulfinic or sulfonic acid moieties. Examples of useful R⁷ also include sulfhydryl-substituted alkyl substituents. Examples of useful R⁷ also include —C(O)NR₂ wherein R is defined as above.

Examples of useful R³ include C₁₋₆ hydrocarbyl residues containing 0-2 heteroatoms independently selected from O, S and N; and inorganic residues. Further examples of useful R³ include halo, alkyl, heteroalkyl, —OC(O)R, —OR, —N(R)C(O)R, —SR, and —NR₂, wherein R is H, alkyl or aryl, or heteroatom-containing forms thereof. Further examples of useful R³ also include alkyl, alkoxy and halo. Further examples of useful R³ also include methyl, methoxy and chloro.

In one embodiment, useful values of n are zero and 1.

In another embodiment, n is zero or n is 1 and R³ is halo or methoxy.

A “linker” is any multivalent radical that is consistent with the Ar-α-ring distance requirement. The portion of the compound between the atom of Ar bound to L² and the center of the α-ring is preferably from 4.5 to 24 Å, preferably less than 24 Å, more preferably less than 20 Å, and still more preferably less than 15 Å. The distance is measured from the center of the α ring to the atom of Ar to which the linker is attached. The identities of linkers L¹ and L² are less important than the distances they impart between portions of the molecule. Examples of useful linkers include —C(O)— and isosteres thereof, or optionally substituted isosteres, or saturated or unsaturated longer chain forms. L¹ or L² may be or may include a heteroatom such as N, S or O.

Examples of useful L¹ include —C(O)—, —S(O)—, —S(O)₂— and —CH(OH)—. In one embodiment, L¹ is —C(O)—.

Examples of useful L² include alkylene or alkenylene optionally substituted with noninterfering substituents. Examples of useful noninterfering substituents include alkyl, alkenyl, alkynyl, aryl, arylalkyl, acyl, aroyl, heteroaryl, heteroalkyl, heteroalkenyl, heteroalkynyl, heteroalkylaryl, —NH-aroyl, halo, —OR, —NR₂, —SR, —S(O)R, —S(O)₂R, —OC(O)R, —N(R)C(O)R, —N(R)C(O)NR₂, —N(R)C(O)OR, —OC(O)NR₂, —C(O)R, —C(O)OR, alkylene-C(O)OR, —S(O)₂OR, —C(O)NR₂, —S(O)₂NR₂, —N(R)S(O)₂NR₂, —CN, —CF₃, —SiR₃ and NO₂, wherein each R is independently H, alkyl, alkenyl or aryl, or heteroatom-containing forms thereof, and wherein two substituents on L² can be joined to form a 3- to 8-membered non-aromatic saturated or unsaturated ring that includes 0-3 heteroatoms selected from O, S and N, or said two substituents can be joined to form a carbonyl moiety or an oxime, oximeether, oximeester or ketal of said carbonyl moiety. Further examples of useful L² include —CH₂—, —CH(CH₃)— and —CH═.

R⁴ represents a noninterfering substituent, e.g., a C₁₋₂₀ hydrocarbyl residue containing 0-5 heteroatoms selected from O, S and N. Examples of useful R⁴ are alkyl, alkoxy, aryl, arylalkyl, aryloxy, heteroalkyl, heteroaryl, heteroarylalkyl, —C(O)R, ═O, acyl, halo, —CN, —OR, —N(R)C(O)R, and —NR₂, where R is H, alkyl (e.g., C₁₋₄ alkyl) or aryl, or heteroatom-containing forms thereof. Each of the R⁴ substituents capable of further substitution is optionally further substituted 1-3 times with substituents independently selected from a group that includes alkyl, alkenyl, alkynyl, aryl, arylalkyl, acyl, aroyl, heteroaryl, heteroalkyl, heteroalkenyl, heteroalkynyl, heteroalkylaryl, —NH-aroyl, halo, —OR, —NR₂, —SR, —S(O)R, —S(O)₂R, —OC(O)R, —N(R)C(O)R, —N(R)C(O)NR₂, —N(R)C(O)OR, —OC(O)NR₂, —C(O)R, —C(O)OR, alkylene-C(O)OR, —S(O)₂OR, —C(O)NR₂, —S(O)₂NR₂, —N(R)S(O)₂NR₂, —CN, —CF₃, —SiR₃ and —NO₂, wherein each R is independently H, alkyl, alkenyl or aryl, or heteroatom-containing forms thereof, and wherein two of R⁴ on adjacent positions can be joined to form a fused, 3- to 8-membered optionally substituted, aromatic or non-aromatic, saturated or unsaturated ring; or R⁴ is ═O or an oxime, oximeether, oximeester or ketal thereof. In one embodiment, R⁴ is C₁₋₄ alkyl and/or ═O. In another embodiment, R⁴ comprises two methyl groups at the 2- and 5-positions or the 3- and 6-positions of a piperidinyl or a piperazinyl ring, or =0 at the 5-position of the ring.

Examples of useful values of m include 0, 1 and 2.

In one embodiment, Z¹ is CH or N.

In one embodiment, l and k are both 1.

Examples of useful noninterfering substituents R⁵ (when Z¹ is R⁵) include halo, alkyl, alkoxy, aryl, arylalkyl, aryloxy, heteroaryl, acyl, carboxyl and hydroxyl. Further examples of useful R⁵ include H, alkyl, —OR, —NR₂, —SR and halo, wherein R is H or alkyl. Additionally, R⁵ can be joined with an R⁴ substituent to form a 3- to 8-membered optionally substituted nonaromatic saturated or unsaturated hydrocarbyl ring containing 0-3 heteroatoms such as O, N and/or S.

Ar is aryl, heteroaryl (including 6-5 fused heteroaryl), cycloaliphatic or cycloheteroaliphatic, any of which can be substituted. In one embodiment, Ar is optionally substituted phenyl. In one embodiment, Ar is substituted at 1 or 2 positions. In another embodiment, Ar is substituted at 1 position. Each substituent on Ar is independently a C₁₋₂₀ hydrocarbyl residue containing 0-5 heteroatoms selected from O, S and N, or is an inorganic residue.

Examples of useful substituents on Ar include alkyl, alkenyl, alkynyl, aryl, arylalkyl, acyl, aroyl, heteroaryl, heteroalkyl, heteroalkenyl, heteroalkynyl, heteroalkylaryl, —NH-aroyl, halo, —OR, —NR₂, —SR, —S(O)R, —S(O)₂R, —OC(O)R, —N(R)C(O)R, —N(R)C(O)NR₂, —N(R)C(O)OR, —OC(O)NR₂, —C(O)R, —C(O)OR, alkylene-C(O)OR, —S(O)₂OR, —C(O)NR₂, —S(O)₂NR₂, —N(R)S(O)₂NR₂, —CN, —CF₃, —SiR₃ and —NO₂, wherein each R is independently H, alkyl, alkenyl or aryl, or heteroatom-containing forms thereof, and wherein two of said optional substituents on adjacent positions can be joined to form a fused 3- to 8-membered optionally substituted, aromatic or nonaromatic, saturated or unsaturated ring. Further examples of useful substituents include halo and C₁₋₄ alkyl. Further examples of useful substituents also include fluoro, chloro and methyl. Those substituents capable of further substitution are optionally further substituted by substituents selected from the preceding list.

The compounds of formula (1) useful for practicing the method of the present invention include not only the free neutral compounds, but also their pharmaceutically acceptable acid-addition salts, including salts of inorganic acids such as hydrochloric, sulfuric, hydrobromic and phosphoric, and salts of organic acids such as acetic tartaric, succinic, benzoic, salicylic and the like. Also included, where the compound of formula (1) contains a carboxyl moiety, are carboxylate salts having a pharmaceutically acceptable cation.

Compounds useful in the practice of the present invention also include, but are not limited to, compounds of formulas:

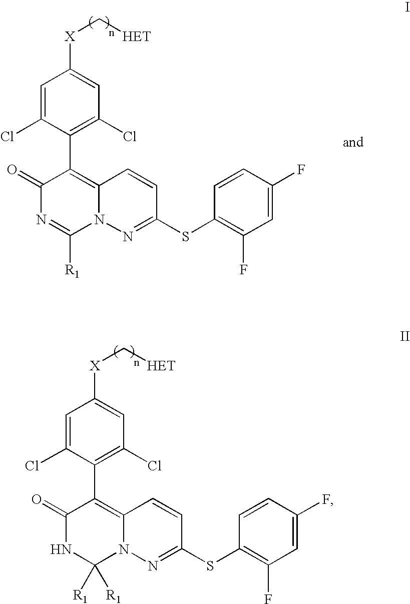

and pharmaceutically acceptable salts thereof,

wherein

HET is a 5-7 membered heterocycle with 1 to 4 N, S or O atoms, which heterocycle is substituted with 1 to 3 C₁-C₄ branched or straight chain alkyl groups. HET is optionally substituted with halo, cyano, N(R′)₂, OR′, CO₂R′, CON(R′)₂ or SO₂N(R²)₂;

X is O or NR′;

n is 1 to 3;

R′is selected from the group consisting of hydrogen, (C₁-C₃)-alkyl, (C₂-C₃)-alkenyl or -alkynyl, phenyl and phenyl substituted with 1 to 3 substituents independently selected from the group consisting of halo, methoxy, cyano, nitro, amino, hydroxy, methyl and ethyl; or is a 5-6 membered heterocyclic ring system optionally substituted with 1 to 3 substituents independently selected from the group consisting of halo, methoxy, cyano, nitro, amino, hydroxy, methyl and ethyl;

R₁ is selected from the group consisting of hydrogen, (C₁-C₃)-alkyl, hydroxy and (C₁-C₃)-alkoxy;

R₂ is selected from the group consisting of hydrogen, (C₁-C₃)-alkyl and (C₁-C₃)-alkenyloxy, each optionally substituted with —N(R′)₂, —OR′, —SR′, —C(O)—N(R′)₂, —S(O₂)—N(R′)₂, —C(O)—OR′ or R³; and

R³ is selected from the group consisting of 5-6 membered aromatic carbocyclic and heterocyclic ring systems.

Compounds useful in the practice of the present invention also include, but are not limited to, compounds of formulas:

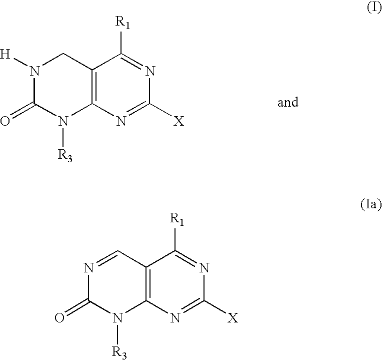

and pharmaceutically acceptable salts thereof,

wherein

R₁ is an aryl or heteroaryl ring, which ring is optionally substituted;

R₂ is hydrogen, C₁₋₁₀ alkyl, C₃₋₇ cycloalkyl, C₃₋₇ cycloalkylC₁₋₁₀ alkyl, aryl, arylC₁₋₁₀ alkyl, heteroaryl, heteroarylC₁₋₁₀ alkyl, heterocyclic or heterocyclylC₁₋₁₀alkyl, each of which, excluding hydrogen, is optionally substituted;

R₃ is C₁₋₁₀ alkyl, C₃₋₇cycloalkyl, C₃₋₇cycloalkylC₁₋₁₀ alkyl, arylC₁₋₁₀ alkyl, heteroarylC₁₋₁₀alkyl or heterocyclylC₁₋₁₀ alkyl, each of which is optionally substituted;

X is R₂, OR₂, S(O)ₘR₂, (CH₂)ₙNR₄R₁₄ or (CH₂)ₙNR₂R₄;

n is 0 or an integer having a value of 1 to 10;

m is 0 or an integer having a value of 1 or 2;

R₄ and R₁₁₄ are independently selected from the group consisting of hydrogen, optionally substituted C₁₋₄ alkyl, optionally substituted aryl and optionally substituted arylC₁₋₄alkyl, or R₄ and R₁₄ together with the nitrogen to which they are attached form a heterocyclic ring of 5 to 7 members, which ring optionally contains an additional heteroatom selected from the group consisting of oxygen, sulfur and NR₉, and which ring is optionally substituted;

R₆ is hydrogen, C₁₋₁₀alkyl, C₃₋₇ cycloalkyl, heterocyclyl, heterocyclylc-IlO alkyl, aryl, arylC₁₋₁₀alkyl, heteroaryl or heteroarylC₁₋₁₀alkyl, each of which, excluding hydrogen, is optionally substituted;

R₉ is hydrogen, C(Z)R₆, optionally substituted C₁₋₁₀ alkyl, optionally substituted aryl or optionally substituted arylC₁₋₄ alkyl;

Z is oxygen or sulfur.

Compounds useful in the practice of the present invention also include, but are not limited to, compounds of formulas:

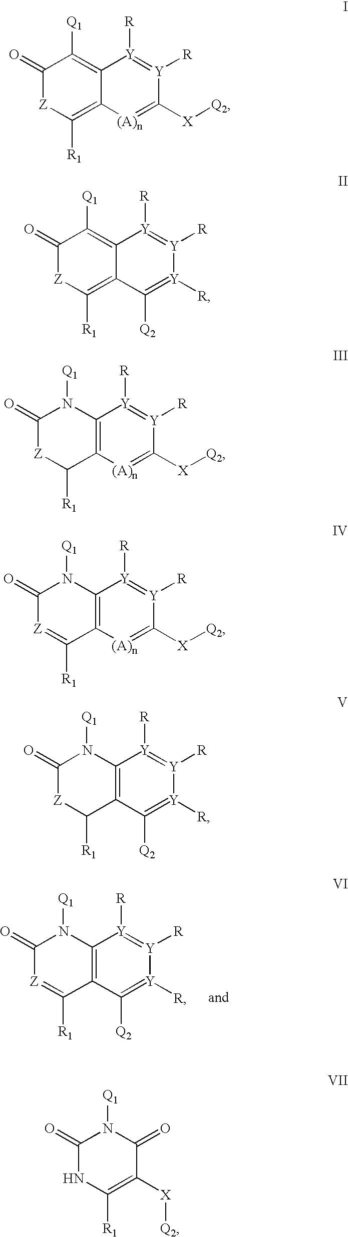

and pharmaceutically acceptable salts thereof,

wherein

Q₁ and Q₂ are independently selected from the group consisting of 5-6 membered aromatic carbocyclic and heterocyclic ring systems and 8-10 membered bicyclic ring systems comprising aromatic carbocyclic rings, aromatic heterocyclic rings and combinations of an aromatic carbocyclic ring and an aromatic heterocyclic ring;

wherein the rings that make up Q, are substituted with 1 to 4 substituents, each of which is independently selected from the group consisting of halo; C₁-C₃ alkyl optionally substituted with NR′₂, OR′, CO₂R′ or CONR′₂; (C₁-C₃)-alkoxy optionally substituted with NR′₂, OR′, CO₂R′ or CONR′₂; NR′₂; OCF₃; CF₃; NO₂; CO₂R′; CONR′; SR′; S(O₂)N(R′)₂; SCF₃; CN; N(R′)C(O)R⁴; N(R′)C(O)OR⁴; N(R′)C(O)C(O)R⁴; N(R′)S(O₂)R⁴; N(R′)R⁴; N(R⁴)₂; OR⁴; OC(O)R⁴; OP(O)₃H₂; and N═C—N(R′)₂;

and wherein the rings that make up Q₂ are optionally substituted with up to 4 substituents, each of which is independently selected from the group consisting of halo; C₁-C₃ straight or branched alkyl optionally substituted with NR′₂, OR′, CO₂R′, S(O₂)N(R′)₂, N═C—N(R′)₂, R³ or CONR′₂; (C₁-C₃)-alkoxy optionally substituted with NR′₂, OR′, CO₂R′, S(O₂)N(R′)₂, N═C—N(R′)₂, R³ or CONR′₂; NR′₂, OCF₃; CF₃; NO₂; CO₂R′; CONR′; R³; OR³; NR³; SR³; C(O)R³; C(O)N(R′)R³; C(O)OR³; SR′; S(O₂)N(R′)₂; SCF₃; N═C—N(R′)₂; and CN;

R′ is selected from the group consisting of hydrogen; (C₁-C₃)-alkyl; (C₂-C₃)-alkenyl; (C₂-C₃)alkynyl; and phenyl substituted with 1 to 3 substituents independently selected from the group consisting of halo, methoxy, cyano, nitro, amino, hydroxy, methyl and ethyl;

R³ is selected from the group consisting of 5-6 membered aromatic carbocyclic and heterocyclic ring systems;

R⁴ is (C₁-C₄)-alkyl optionally substituted with N(R′)₂, OR′, CO₂R′, CON(R′)₂ or SO₂N(R²)₂; or is a 5-6 membered carbocyclic or heterocyclic ring system optionally substituted with N(R′)₂, OR′, CO₂R′, CON(R′)₂ or SO₂N(R²)₂;

X, if present, is selected from the group consisting of —S—, —O—, —S(O₂)—, —S(O)—, —S(O₂)—N(R²)—, —N(R²)—S(O₂)—, —N(R₂)—C(O)O—, —O—C(O)—N(R²), —C(O)—, —C(O)O—, —O—C(O)—, —C(O)—N(R²)—, —N(R²)—C(O)—, —N(R²)—, —C(R₂)₂— and —C(OR²)₂—;

each R is independently selected from the group consisting of hydrogen, —R₂, —N(R₂)₂, —OR², SR₂, —C(O)—N(R²)₂, —S(O₂)—N(R²)₂ and —C(O)—OR², wherein two adjacent R are optionally bound to one another and, together with each Y to which they are respectively bound, form a 4-8 membered carbocyclic or heterocyclic ring;

R² is hydrogen, (C₁-C₃)-alkyl or (C₁-C₃)-alkenyl, each of which is optionally substituted with —N(R′)₂, —OR′, SR′, —C(O)—N(R′)₂, —S(O₂)—N(R′)₂, —C(O)—OR′ or R³;

Y is N or C;

Z, if present, is N, NH or, if chemically feasible, O;

A, if present, is N or CR′;

n is 0 or 1; and

R₁ is from hydrogen, (C₁-C₃)-alkyl, hydroxy or (C₁-C₃)-alkoxy.

Compounds useful in the practice of the present invention also include, but are not limited to, compounds of formula:

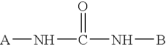

and pharmaceutically acceptable salts thereof,

wherein A is

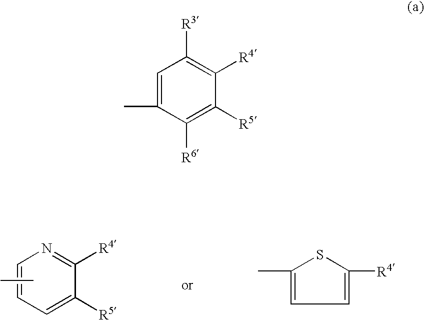

wherein

R³′, R⁴, R⁵ are independently H. C₁-10 -alkyl optionally substituted by halogen up to perhalo, C₁₋₁₀alkoxy optionally substituted by halogen up to perhaloalkoxy, halogen, NO₂ or NH₂;

R^{6′} is H, C₁₋₁₀-alkyl, C₁₋₁₀alkoxy, —NHCOR¹, —NR¹COR¹, NO₂,

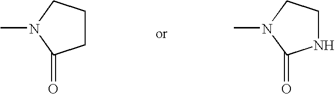

one of R^{4′}, R^{5′}, or R^{6′} can be —X—Y; or 2 adjacent R⁴′-R⁶′ can together be an aryl or heteroaryl ring with 5-12 atoms, optionally substituted by C₁₋₁₀-alkyl, C₁₋₁₀alkoxy, C₃₋₁₀ cycloalkyl, C₂₋₁O alkenyl, C₁₋₁₀ alkanoyl, C₆₋₁₂ aryl, C₅₋₁₂ heteroaryl or C₆₋₁₂ arakyl;

R¹ is C₁₋₁₀-alkyl optionally substituted by halogen, up to perhalo;

X is —CH₂—, —S—, —N(CH₃)—, —NHC(O)—, —CH₂—S—, —S—CH₂—, —C(O)— or —O—;

X is additionally a single bond where Y is pyridyl;

Y is phenyl, pyridyl, naphthyl, pyridone, pyrazine, benzodioxane, benzopyridine, pyrimidine or benzothiazole, each optionally substituted by C₁₋₁₀-alkyl, C₁₋₁₀-alkoxy, halogen, OH, —SCH₃ or NO₂ or, where Y is phenyl, by

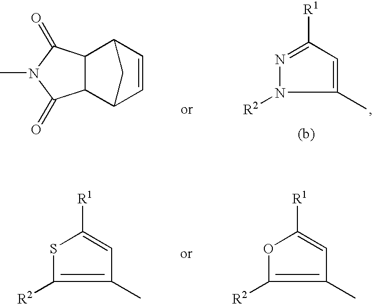

wherein

R¹ is selected from the group consisting of C₃-C₁₀ alkyl, C₃-C₁₀ cycloalkyl, up to per-halo substituted C¹-C₁₀ alkyl and up to per-halosubstituted C₃-C₁₀ cycloalkyl; and

R₂ is C₆-C₁₄ aryl, C₃-C₁₄ heteroaryl, substituted C₆-C₁₄ aryl or substituted C₃-C₁₄ heteroaryl;

wherein if R² is a substituted group, it is preferably substituted by one or more substituents independently selected from the group consisting of halogen, up to per-halosubstitution, and Vₙ, where n=0-3 and each V is independently selected from the group consisting of-CN, —OC(O)NR⁵R^{5′}, —CO₂R⁵, —C(O)NR⁵R⁵, —OR⁵, —SR⁵, —NR⁵R_{5′}, —C(O)R⁵, —NR⁵C(O)OR^{5′}, —SO₂R⁵—SOR⁵, —NR⁵C(O)R^{5′}, —NO₂, C₁-C₁₀ alkyl, C₃-C₁₀ cycloalkyl, C₆-C₁₄ aryl, C₃-C₁₃ heteroaryl, C₇-C₂₄ alkaryl, C₄-C₂₄ alkheteroaryl, substituted C₁-C₁₀ alkyl, substituted C₃-C₁₀ cycloalkyl, substituted C₆-C₁₄ aryl, substituted C₃-C₁₃ heteroaryl, substituted C₇-C₂₄ alkaryl and substituted C₄-C₂₄ alkheteroaryl;

wherein if V is a substituted group, it is substituted by one or more substituents independently selected from the group consisting of halogen, up to per-halosubstitution, —CN, —CO₂R⁵, —C(O)R⁵, —C(O)NR⁵R^{5′}, —NR⁵R^{5′}, —OR⁵, —SR⁵, —NR⁵C(O)R^{5′}, —NR⁵C(O)OR^{5′} and —NO₂; and

R⁵ and R^{5′} are independently selected from the group consisting of H, C₁-C₁₀ alkyl, C₃-C₁₀ cycloalkyl, C₆-C₁₄ aryl, C₃-C₁₃ heteroaryl, C₇-C₂₄ alkaryl, C₄-C₂₃ alkheteroaryl, up to per-halosubstituted C₁-C₁₀ alkyl, up to per-halosubstituted C₃-C₁₀ cycloalkyl, up to per-halosubstituted C₆-C₁₄ aryl and up to per-halosubstituted C₃-C₁₃ heteroaryl; or

(c) a substituted moiety of up to 40 carbon atoms of the formula: -L-(M-L¹)_{q}, where L is a 5- or 6-membered cyclic structure bound directly to the nitrogen atom of —NHC(O)NH—B, L¹ comprises a substituted cyclic moiety having at least 5 members, M is a bridging group having at least one atom, q is an integer of from 1-3; and each cyclic structure of L and L¹ contains 0-4 members of the group consisting of nitrogen, oxygen and sulfur;

L¹ is substituted by at least one substituent selected from the group consisting of —SO₂Rₓ, —C(O)Rₓ and —C(NR_{y})R_{z};

R_{y} is hydrogen or a carbon-based moiety of up to 24 carbon atoms optionally containing heteroatoms selected from the group consisting of N, S and O, and optionally halosubstituted, up to perhalo;

R_{z} is hydrogen or a carbon-based moiety of up to 30 carbon atoms optionally containing heteroatoms selected from the group consisting of N, S and O, and optionally substituted by halogen, hydroxy and carbon-based substituents of up to 24 carbon atoms, which optionally contain heteroatoms selected from the group consisting of N, S and O, and are optionally substituted by halogen; and

R, is R_{z} or NRₐR_{b} where Rₐ and R_{b} are

i) independently hydrogen,

a carbon-based moiety of up to 30 carbon atoms optionally containing heteroatoms selected from the group consisting of N, S and O, and optionally substituted by halogen, hydroxy and carbon-based substituents of up to 24 carbon atoms, which optionally contain heteroatoms selected from the group consisting of N, S and O, and are optionally substituted by halogen, or

—OSi(R_{f})₃ where R_{f} is hydrogen or a carbon-based moiety of up to 24 carbon atoms optionally containing heteroatoms selected from the group consisting of N, S and O, and optionally substituted by halogen, hydroxy and carbon-based substituents of up to 24 carbon atoms, which optionally contain heteroatoms selected from the group consisting of N, S and O, and are optionally substituted by halogen; or

ii) Rₐ and R_{b} together form a 5-7 member heterocyclic structure of 1-3 heteroatoms selected from the group consisting of N, S and O, or a substituted 5-7 member heterocyclic structure of 1-3 heteroatoms selected from the group consisting of N, S and O, substituted by halogen, hydroxy or carbon-based substituents of up to 24 carbon atoms, which optionally contain heteroatoms selected from the group consisting of N, S and O, and are optionally substituted by halogen; or

iii) one of Rₐ or R_{b} is —C(O)—, a C₁-C₅ divalent alkylene group or a substituted C₁-C₅ divalent alkylene group bound to the moiety L to form a cyclic structure with at least 5 members, wherein the substituents of the substituted C I—C₅ divalent alkylene group are selected from the group consisting of halogen, hydroxy, and carbon-based substituents of up to 24 carbon atoms, which optionally contain heteroatoms selected from N, S and O and are optionally substituted by halogen; and

B is an unsubstituted or substituted, up to tricyclic, aryl or heteroaryl moiety with up to 30 carbon atoms with at least one 5- or 6-membered aromatic structure containing 0-4 members of the group consisting of nitrogen, oxygen and sulfur;

wherein if B is substituted, it is substituted by one or more substituents selected from the group consisting of halogen, up to per-halo, and Wₙ, wherein

n is 0-3 and each W is independently selected from the group consisting of

—CN, —CO₂R⁷, —C(O)NR⁷R⁷, —C(O)R⁷, —NO₂, —OR⁷, —SR⁷, —NR⁷R⁷, —NR⁷C(O)OR⁷, —NR⁷C(O)R⁷, C₁-C₁₀ alkyl, C₂₋₁₀-alkenyl, C₁₋₁₀-alkoxy, C₃-C₁₀ cycloalkyl, C₆-C₁₄ aryl, C₇-C₂₄ alkaryl, C₃-C₁₃ heteroaryl, C₄-C₂₃ alkheteroaryl, substituted C₁-C₁₀ alkyl, substituted C₂₋₁₀-alkenyl, substituted C₁₋₁₀ alkoxy, substituted C₃-C₁₀ cycloalkyl, substituted C₄-C₂₃ alkheteroaryl and -Q-Ar;

wherein if W is a substituted group, it is substituted by one or more substituents independently selected from the group consisting of —CN, —CO₂R⁷, —C(O)NR⁷R⁷, —C(O)R⁷, —NO₂, —OR⁷, —SR⁷, —NR⁷R⁷, —NR⁷C(O)OR⁷, —NR⁷C(O)R⁷ and halogen up to per-halo;

wherein each R⁷ is independently selected from the group consisting of H, C₁-C₁₀ alkyl, C₂₋₁₀ alkenyl, C₃-C₁₀ cycloalkyl, C₆-C₁₄ aryl, C₃-C₁₃ heteroaryl, C₇-C₂₄ alkaryl, C₄-C₂₃ alkheteroaryl, up to per-halosubstituted C₁-C₁₀ alkyl, up to per-halosubstituted C₂₋₁₀ alkenyl, up to per-halosubstituted C₃-C₁₀ cycloalkyl, up to per-halosubstituted C₆-C₁₄ aryl and up to per-halosubstituted C₃-C₁₃ heteroaryl;

wherein Q is —O—, —S—, —N(R)⁷, —(CH₂)—ₘ, —C(O)—, —CH(OH)—, —NR⁷C(O)NR⁷R⁷—, —NR⁷C(O)—, —C(O)NR⁷—, —(CH₂)ₘO—, —(CH₂)ₘS—, —(CH₂)ₘN(R⁷)—, —O(CH₂)ₘ—, —CHX^{a}, —CX^{a}₂—, —S—(CH₂)ₘ— or —N(R⁷)(CH₂)ₘ—, where m=1-3, and Xa is halogen; and

Ar is a 5-10 member aromatic structure containing 0-4 members of the group consisting of nitrogen, oxygen and sulfur, which is unsubstituted or substituted by halogen up to per-halosubstitution and optionally substituted by Zₙ₁, wherein n1 is 0 to 3 and each Z substituent is independently selected from the group consisting of —CN, —CO₂R⁷, —C(O)NR⁷R⁷, —C(O)—NR⁷, —NO₂, —OR⁷, —SR⁷, —NR⁷R⁷, —NR⁷C(O)OR⁷, —C(O)R⁷, —NR⁷C(O)R⁷, C₁-C₁₀ alkyl, C₃-C₁₀ cycloalkyl, C₆-C₁₄ aryl, C₃-C₁₋₃ heteroaryl, C₇-C₂₄ alkaryl, C₄-C₂₃ alkheteroaryl, substituted C₁-C₁₀ alkyl, substituted C₃-C₁₀ cycloalkyl, substituted C₇-C₂₄ alkaryl and substituted C₄-C₂₃ alkheteroaryl; wherein the one or more substituents of Z are independently selected from the group consisting of —CN, —CO₂R⁷, —C(O)NR⁷R⁷, —OR⁷, —SR⁷, —NO₂, —NR⁷R⁷, —NR⁷C(O)R⁷ and —NR⁷C(O)OR⁷.

Exemplary compounds of these formulas include:

N-(5-tert-butyl-2-methoxyphenyl)-N′-(4-phenyloxyphenyl)urea;

N-(5-tert-butyl-2-methoxyphenyl)-N′-(4-(4-methoxyphenyloxy)phenyl)urea;

N-(5-tert-butyl-2-methoxyphenyl)-N′-(4-(4-pyridinyloxy)phenyl)urea;

N-(5-tert-butyl-2-methoxyphenyl)-N′-(4-(4-pyridinylmethyl)phenyl)urea;

N-(5-tert-butyl-2-methoxyphenyl)-N′-(4-(4-pyridinylthio)phenyl)urea;

N-(5-tert-butyl-2-methoxyphenyl)-N′-(4-(4-(4,7-methano-1H-isoindole-1, 3 (2H)-dionyl)methyl)phenyl)urea;

N-(5-tert-butyl-2-phenylphenyl)-N′-(2,3-dichlorophenyl)urea;

N-(5-tert-butyl-2-(3-thienyl)phenyl)-N′-(2,3-dichlorophenyl)urea;

N-(5-tert-butyl-2-(N-methylaminocarbonyl)methoxyphenyl)-N′-(2,3-dichlorophenyl)urea;

N-(5-tert-butyl-2-(N-methylaminocarbonyl)methoxyphenyl)-N′-(1-naphthyl)urea;

N-(5-tert-butyl-2-(N-morpholinocarbonyl)methoxyphenyl)-N′-(2,3-dichlorophenyl)urea;

N-(5-tert-butyl-2-(N-morpholinocarbonyl)methoxyphenyl)-N′-(1-naphthyl)urea;

N-(5-tert-butyl-2-(3-tetrahydrofuranyloxy)phenyl)-N′-(2,3-dichlorophenyl)urea;

N-(5-tert-butyl-2-methoxyphenyl)-N′-(4-(3-pyridinyl)methylphenyl)urea;

N-(5-trifluoromethyl-2-methoxyphenyl)-N′-(4-methylphenyl)urea; N-(5-trifluoromethyl-2-mrethoxyphenyl)-N′-(4-methyl-2-fluorophenyl)urea;

N-(5-trifluoromethyl-2-methoxyphenyl)-N′-(4-fluoro-3-chlorophenyl)urea;

N-(5-trifluoromethyl-2-methoxyphenyl)-N′-(4-methyl-3-chlorophenyl)urea;

N-(5-trifluoromethyl-2-methoxyphenyl)-N′-(4-methyl-3-fluorophenyl)urea;

N-(5-trifluoromethyl-2-methoxyphenyl)-N′-(2,4-difluorophenyl)urea;

N-(5-trifluoromethyl-2-methoxyphenyl)-N′-(4-phenyloxy-3,5-dichlorophenyl)urea;

N-(5-trifluoromethyl-2-methoxyphenyl)-N′-(4-(4-pyridinylmethyl)phenyl)urea;

N-(5-trifluoromethyl-2-methoxyphenyl)-N′-(4-(4-pyridinylthio)phenyl)urea;

N-(5-trifluoromethyl-2-methoxyphenyl)-N′-(4-(4-pyridinyloxy)phenyl)urea;

N-(5-trifluoromethyl-2-methoxyphenyl)-N′-(3-(4-pyridinylthio)phenyl)urea;

N-(5-trifluoromethyl-2-methoxyphenyl)-N′-(4-(3-(N-methylaminocarbonyl)phenyloxy)phenyl)urea;

N-(5-fluorosulfonyl)-2-methoxyphenyl)-N ′-(4-methylphenyl)urea;

N-(5-(difluromethanesulfonyl)-2-methoxyphenyl)-N′-(4-methylphenyl)urea;

N-(5-(difluoromethanesulfonyl)-2-methoxyphenyl)-N′-(4-fluorophenyl)urea;

N-(5-(difluoromethanesulfonyl)-2-methoxyphenyl)-N′-(4-methyl-2-fluorophenyl)urea;

N-(5-(difluoromethanesulfonyl)-2-methoxyphenyl)-N′-(4-methyl-3-fluorophenyl)urea;

N-(5-(difluoromethanesulfonyl)-2-methoxyphenyl)-N′-(4-methyl-3-chlorophenyl)urea;

N-(5-(difluoromethanesulfonyl)-2-methoxyphenyl)-N′-(4-fluoro-3-chlorophenyl)urea;

N-(5-(difluoromethanesulfonyl)-2-methoxyphenyl)-N′-(4-fluoro-3-methylphenyl)urea;

N-(5-(difluoromethanesulfonyl)-2-methoxyphenyl)-N′-(2,3-dimethylphenyl)urea;

N-(5-(trifluoromethanesulfonyl)-2-methoxphenyl)-N′-(4-methylphenyl)urea;

N-(3-methoxy-2-naphthyl)-N′-(2-fluorophenyl)urea;

N-(3-methoxy-2-naphthyl)-N′-(4-methylphenyl)urea;

N-(3-methoxy-2-naphthyl)-N′-(3-fluorophenyl)urea;

N-(3-methoxy-2-naphthyl)-N′-(4-methyl-3-fluorophenyl)urea;

N-(3-methoxy-2-naphthyl)-N′-(2,3-dimethylphenyl)urea;

N-(3-methoxy-2-naphthyl)-N′-(1-naphthyl)urea;

N-(3-methoxy-2-naphthyl)-N′-(4-(4-pyridinylmethyl)phenyl)urea;

N-(3-methoxy-2-naphthyl)-N′-(4-(4-pyridinylthio)phenyl)urea;

N-(3-methoxy-2-naphthyl)-N ′-(4-(4-methoxyphenyloxy)phenyl)urea;

N-(3-methoxy-2-naphthyl)-N′-(4-(4-(4,7-methano-1H-isoindole-1,3-(2H)-dionyl)methyl)phenyl)urea;

N-(2-hydroxy-4-nitro-5-chlorophenyl)-N′-(phenyl)urea; and

N-(2-hydroxy-4-nitro-5-chlorophenyl)-N′-(4-(4-pyridinylmethyl)phenyl)urea;

and pharmaceutically acceptable salts thereof.

Such compounds are described in published PCT applications WO 96/21452, WO 96/40143, WO 97/25046, WO 97/35856, WO 98/25619, WO 98/56377, WO 98/57966, WO 99/32110, WO 99/32121, WO 99/32463, WO 99/61440, WO 99/64400, WO 00/10563, WO 00/17204, WO 00/19824, WO 00/41698, WO 00/64422, WO 00/71535, WO 01/38324, WO 01/64679, WO 01/66539, and WO 01/66540, each of which is herein incorporated by reference.

Also for useful in the practice of the present invention are compositions comprising a compound of any of the formulas above, where the substituents are defined as above following each formula, or a pharmaceutically acceptable salt thereof, and one or more pharmaceutically acceptable excipients.

In all instances herein where there is an alkenyl or alkynyl moiety as a substituent group, the unsaturated linkage, i.e., the vinylene or acetylene linkage, is preferably not directly attached to the nitrogen, oxygen or sulfur moieties, for instance in OR_{f}, or for certain R₂ moieties.

As used herein, “optionally substituted” unless specifically defined shall mean such groups as halogen, such as fluorine, chlorine, bromine or iodine; hydroxy; hydroxy-substituted C₁₋₁₀alkyl; C₁₋₁₀alkoxy, such as methoxy or ethoxy; S(O)ₘ alkyl, wherein m is 0, 1 or 2, such as methyl thio, methylsulfinyl or methyl sulfonyl; amino, mono and di-substituted amino, such as in the NR₇R₁₇ group; or where the R₇R₁₇ can together with the nitrogen to which they are attached cyclize to form a 5- to 7-membered ring which optionally includes an additional heteroatom selected from O, N, and S; C₁₋₁₀ alkyl, cycloalkyl, or cycloalkyl alkyl group, such as methyl, ethyl, propyl, isopropyl, t-butyl, etc. or cyclopropyl methyl; halo-substituted C₁₋₁₀alkyl, such as CF₃; an optionally substituted aryl, such as phenyl, or an optionally substituted arylalkyl, such as benzyl or phenethyl, wherein these aryl moieties can also be substituted one to two times by halogen; hydroxy; hydroxy-substituted alkyl; C₁₋₁₀alkoxy; S(O)ₘ alkyl; amino, mono- and di-substituted amino, such as in the NR₇R₁₇ group; alkyl, or CF₃.

Inhibitors useful in the present invention can be used with any pharmaceutically acceptable salt. The term “pharmaceutically acceptable salts” refers to salts prepared from pharmaceutically acceptable non-toxic bases or acids. When the compound utilized by the present invention is acidic, its corresponding salt can be conveniently prepared from pharmaceutically acceptable non-toxic bases, including inorganic bases and organic bases. Salts derived from such inorganic bases include aluminum, ammonium, calcium, copper (ic and ous), ferric, ferrous, lithium, magnesium, manganese (ic and ous), potassium, sodium, zinc and the like salts. Particularly preferred are the ammonium, calcium, magnesium, potassium and sodium salts. Salts derived from pharmaceutically acceptable organic non-toxic bases include salts of primary, secondary, and tertiary amines, as well as cyclic amines and substituted amines such as naturally occurring and synthesized substituted amines. Basic salts of inorganic and organic acids also include as hydrochloric acid, hydrobromic acid, sulphuric acid, phosphoric acid, methane sulphonic acid, ethane sulphonic acid, acetic acid, malic acid, tartaric acid, citric acid, lactic acid, oxalic acid, succinic acid, fumaric acid, maleic acid, benzoic acid, salicylic acid, phenylacetic acid and mandelic acid. In addition, pharmaceutically-acceptable salts of the above-described compounds can also be formed with a pharmaceutically-acceptable cation, for instance, if a substituent group comprises a carboxy moiety. Suitable pharmaceutically-acceptable cations are well known to those skilled in the art and include alkaline, alkaline earth, ammonium and quaternary ammonium cations.

Other pharmaceutically acceptable organic non-toxic bases from which salts can be formed include ion exchange resins such as, for example, arginine, betaine, caffeine, choline, N,N-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like.

The inhibitors of p38 MAP kinase can be used as single therapeutic agents or in combination with other therapeutic agents. Drugs that could be usefully combined with these compounds include monoclonal antibodies targeting cells of the immune system, antibodies or soluble receptors or receptor fusion proteins targeting immune or non-immune cytokines, and small molecule inhibitors of cell division, protein synthesis, or mRNA transcription or translation, or inhibitors of immune cell differentiation, activation, or function (e.g., cytokine secretion).

The following terms, as used herein, refer to:

“halo” or “halogens”, include the halogens: chloro, fluoro, bromo and iodo;

“C₁₋₁₀alkyl” or “alkyl”—both straight and branched chain radicals of 1 to 10 carbon atoms, unless the chain length is otherwise limited, including, but not limited to, methyl, ethyl, n-propyl, iso-propyl, n-butyl, sec-butyl, iso-butyl, tert-butyl, n-pentyl and the like;

the term “cycloalkyl” is used herein to mean cyclic radicals, preferably of 3 to 8 carbons, including but not limited to cyclopropyl, cyclopentyl, cyclohexyl, and the like;

the term “cycloalkenyl” is used herein to mean cyclic radicals, preferably of 5 to 8 carbons, which have at least one double bond, including but not limited to cyclopentenyl, cyclohexenyl, and the like;

the term “alkenyl” is used herein at all occurrences to mean straight or branched chain radical of 2-10 carbon atoms, unless the chain length is limited thereto, wherein there is at least one double bond between two carbon atoms in the chain, including, but not limited to ethenyl, 1-propenyl, 2-propenyl, 2-methyl-1-propenyl, 1-butenyl, 2-butenyl and the like;

“aryl”—phenyl and naphthyl;

“heteroaryl” (on its own or in any-combination, such as “heteroaryloxy” or “heteroaryl alkyl”)—a 5-10-membered aromatic ring system in which one or more rings contain one or more heteroatoms selected from the group consisting of N, O and S, such as, but not limited, to pyrrole, pyrazole, furan, thiophene, quinoline, isoquinoline, quinazolinyl, pyridine, pyrimidine, oxazole, thiazole, thiadiazole, triazole, imidazole, or benzimidazole;

“heterocyclic” (on its own or in any combination, such as “heterocyclylalkyl”)—a saturated or partially unsaturated 4-10-membered ring system in which one or more rings contain one or more heteroatoms selected from the group consisting of N, O, and S; such as, but not limited to, pyrrolidine, piperidine, piperazine, morpholine, tetrahydropyran, or imidazolidine;

the term “aralkyl” or “heteroarylalkyl” or “heterocyclicalkyl” is used herein to mean C₁₋₄ alkyl as defined above attached to an aryl, heteroaryl or heterocyclic moiety as also defined herein unless otherwise indicate;

“sulfinyl”—the oxide S(O) of the corresponding sulfide, the term “thio” refers to the sulfide, and the term “sulfonyl” refers to the fully oxidized S(O)₂ moiety;

“aroyl”—a C(O)Ar, wherein Ar is as phenyl, naphthyl, or aryl alkyl derivative such as defined above, such groups include but are not limited to benzyl and phenethyl; and

“alkanoyl”—a C(O)C₁₋₁₀ alkyl wherein the alkyl is as defined above.

For the purposes herein the “core” 4-pyrimidinyl moiety for R₁ or R₂ is referred to as the formula:

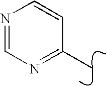

The compounds useful in the practice of the present invention can contain one or more asymmetric carbon atoms and can exist in racemic and optically active forms. The use of all of these compounds are included within the scope of the present invention.

Compounds useful in the practice of the present invention also include, but are not limited to, the compounds shown in Tables A and B, below.

TABLE A

| | Citations, each of which is herein |
|---|---|
| Chemical Structure | incorporated by reference. |
| 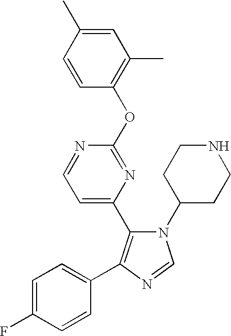 | WO-00166539, WO-00166540, WO-00164679, WO-00138324, WO-00064422, WO-00019824, WO-00010563, WO-09961440, WO-09932121, WO-09857966, WO-09856377, WO-09825619, WO-05756499, WO-09735856, WO-09725046, WO-09640143, WO-09621452; Gallagher, T.F., et. al., Bioorg. Med. Chem. 5:49 (1997); Adams, J.L., et al., Bioorg. Med. Chem. Lett. 8:3111-3116 (1998) |
| 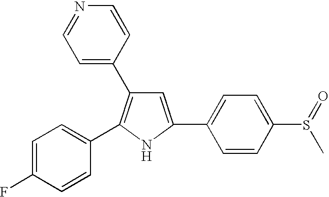 | De Laszlo, S.E., et. Al., Bioorg Med Chem Lett. 8:2698 (1998) |
| 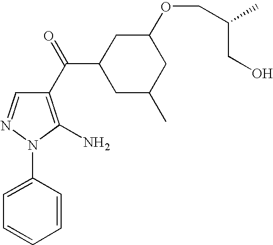 | WO-09957101; Poster presentation at the 5^{th} World Congress on Inflammation, Edinburgh, UK. (2001) |
| 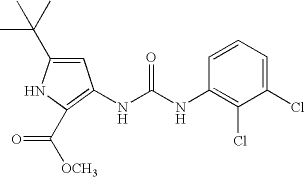 | WO-00041698, WO-09932110, WO-09932463 |
| 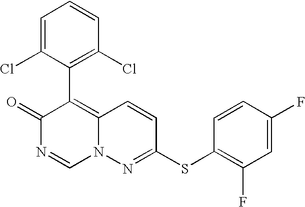 | WO-00017204, WO-09964400 |
| 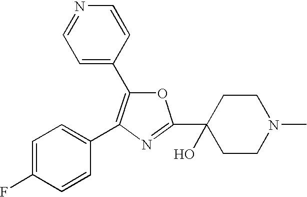 | Revesz. L., et. al., Bioorg Med Chem Lett. 10:1261 (2000) |
| 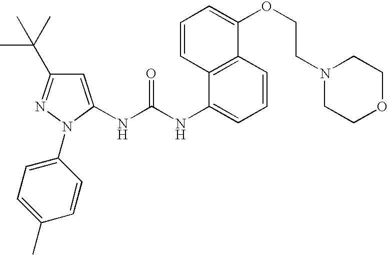 | WO-00207772 |
| 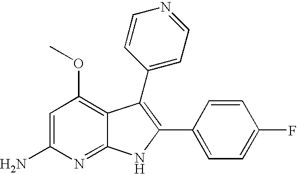 | Fijen, J.W., et al., Clin. Exp. Immunol. 124:16-20 (2001); Wadsworth, S.A., et. al., J. Pharmacol. Expt. Therapeut. 291:680 (1999) |
| 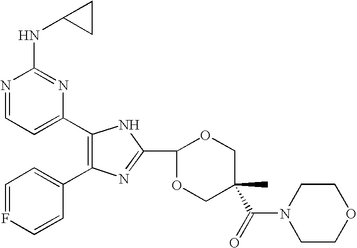 | Collis, A.J., et al.. Bioorg. Med. Chem. Lett. 11:693-696 (2001); McLay, L.M., et al., Bioorg Med Chem 9:537-554 (2001) |
| 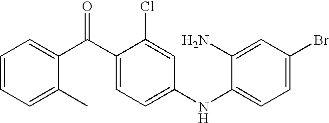 | WO-00110865,WO-00105749 |

TABLE B

| Compd. # | STRUCTURE |
|---|---|
| 1 | 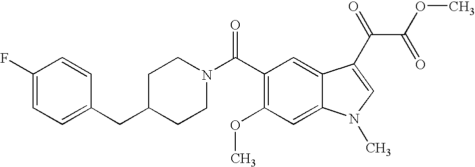 |
| 2 | 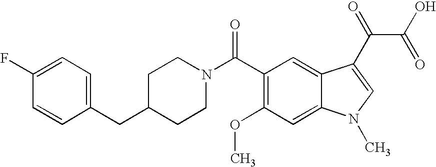 |
| 3 | 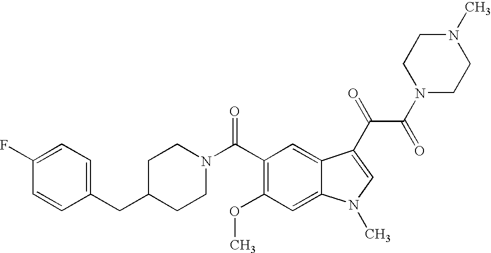 |
| 4 | 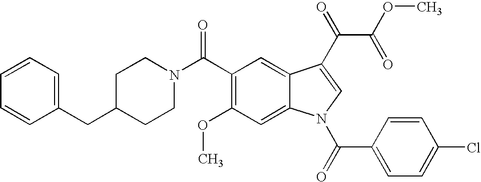 |
| 5 | 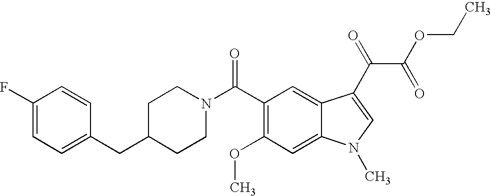 |
| 6 | 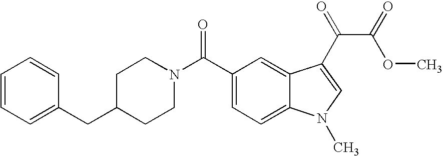 |
| 7 | 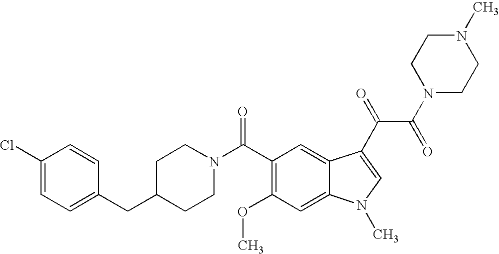 |
| 8 | 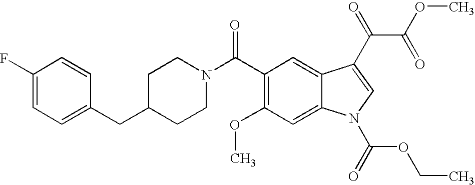 |
| 9 | 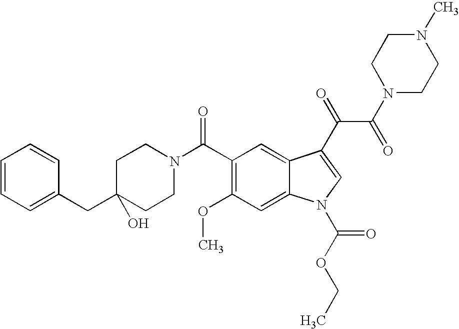 |
| 10 | 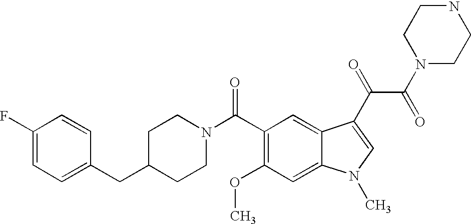 |
| 11 | 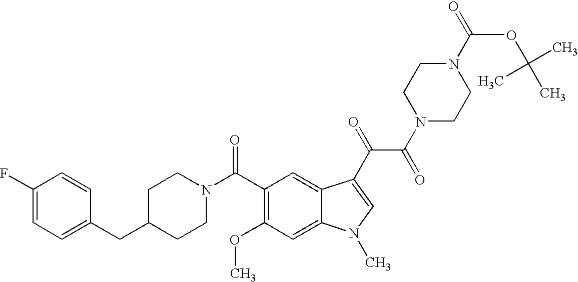 |
| 12 | 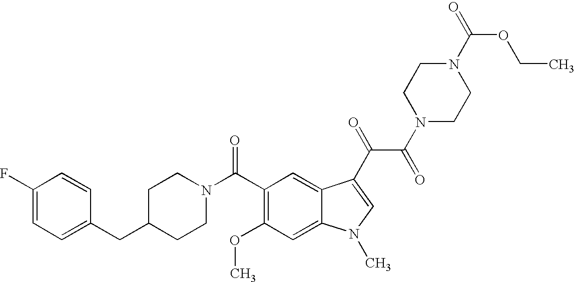 |
| 13 | 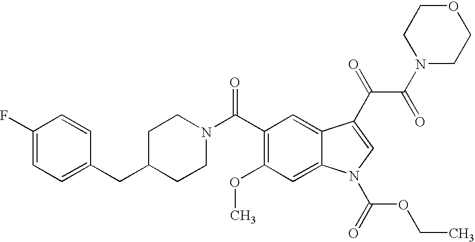 |
| 14 | 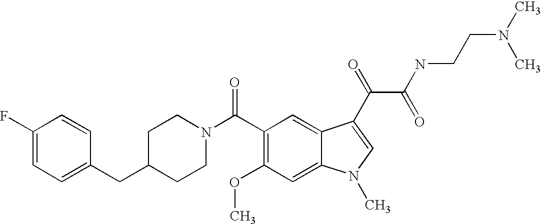 |
| 15 | 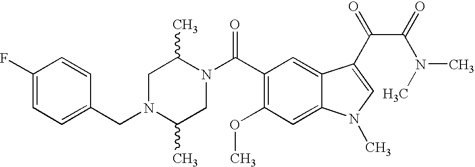 |
| 16 | 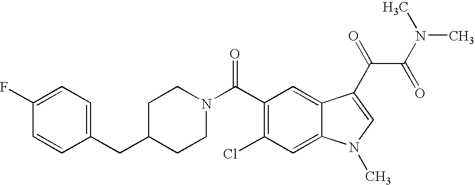 |
| 17 | 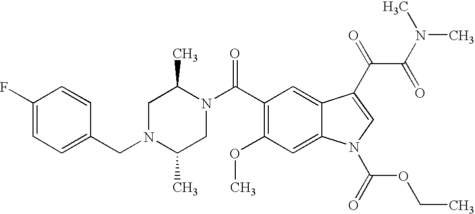 |
| 18 | 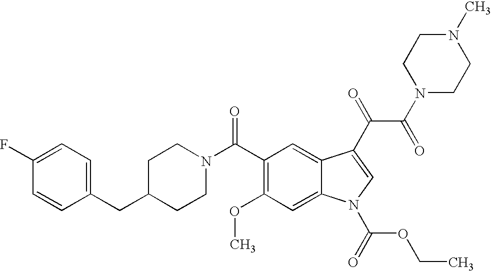 |
| 19 | 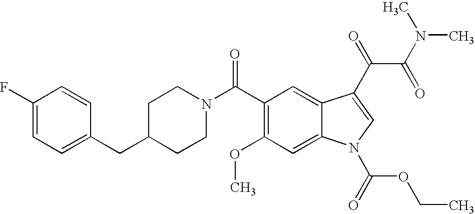 |
| 20 | 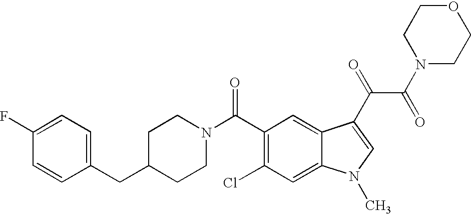 |
| 21 | 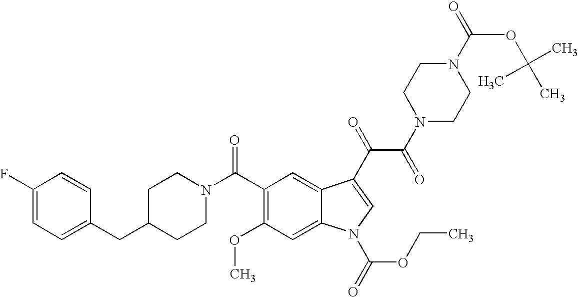 |
| 22 | 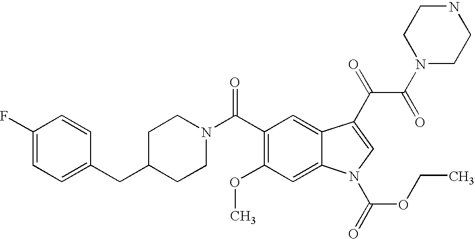 |
| 23 | 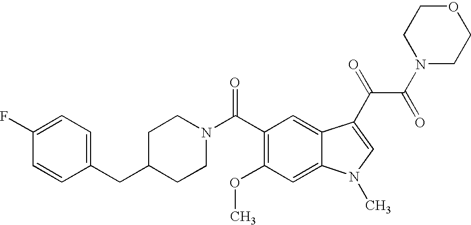 |
| 24 | 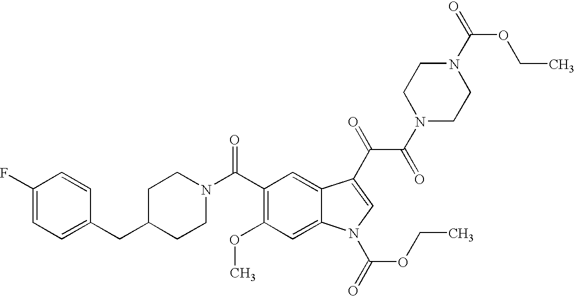 |
| 25 | 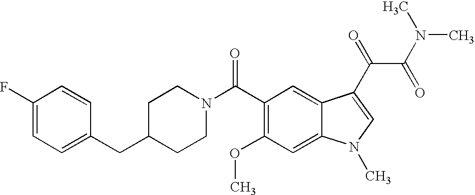 |
| 26 | 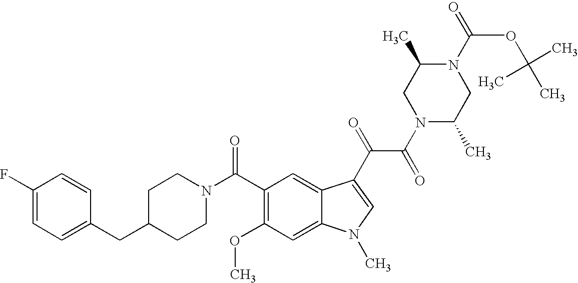 |
| 27 | 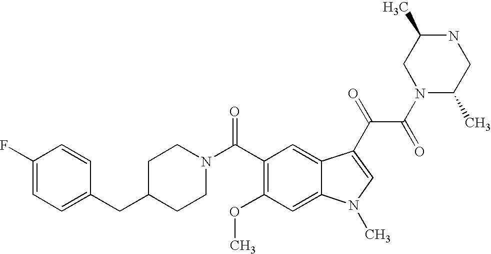 |
| 28 | 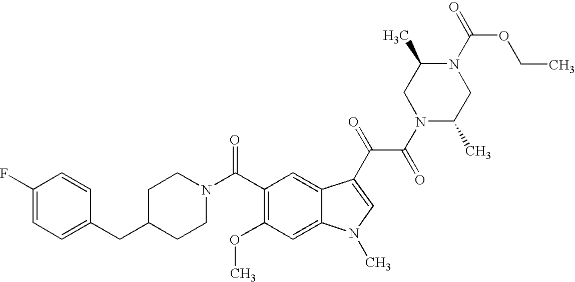 |
| 29 | 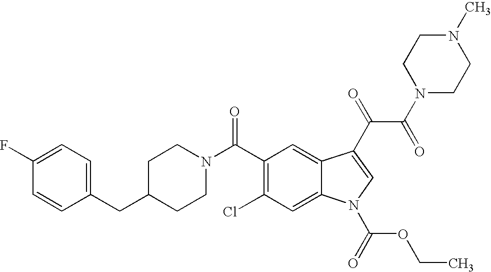 |
| 30 | 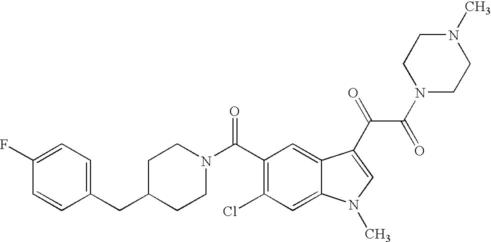 |
| 31 | 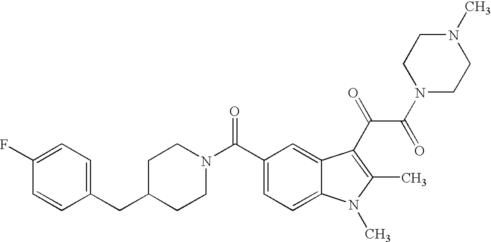 |
| 32 | 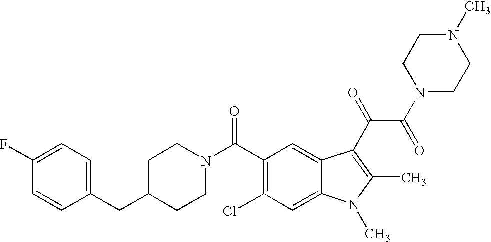 |
| 33 | 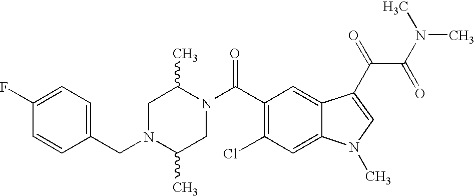 |
| 34 | 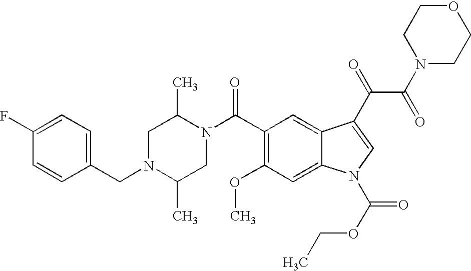 |
| 35 | 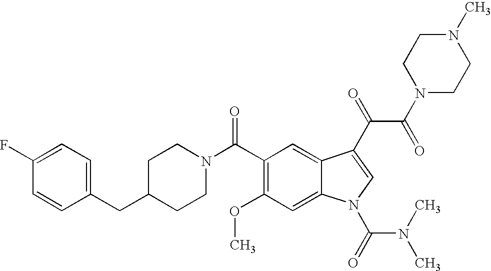 |
| 36 | 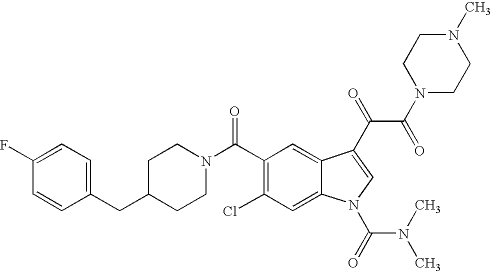 |
| 37 | 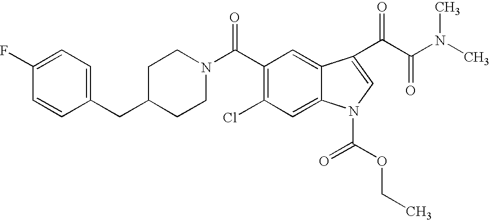 |
| 38 | 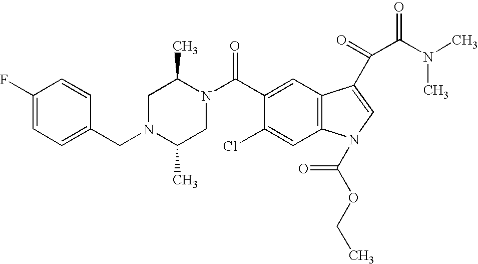 |
| 39 | 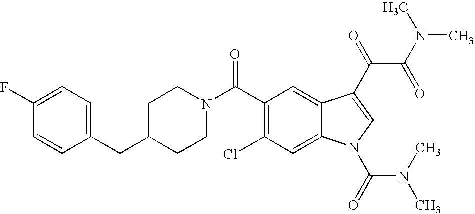 |
| 40 | 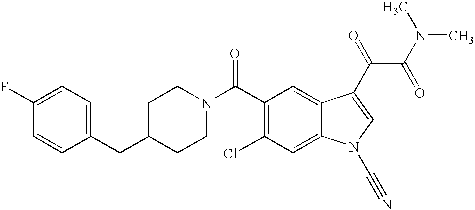 |
| 41 | 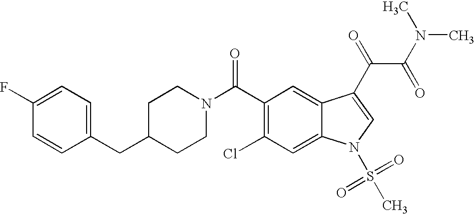 |
| 42 | 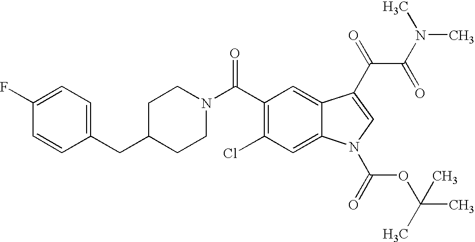 |
| 43 | 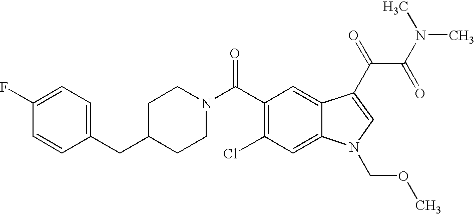 |
| 44 | 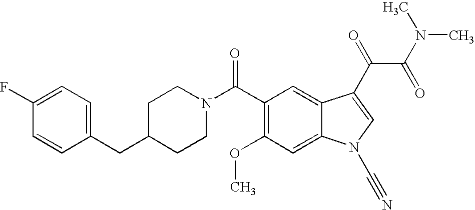 |
| 45 | 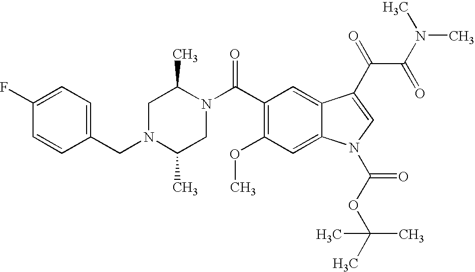 |
| 46 | 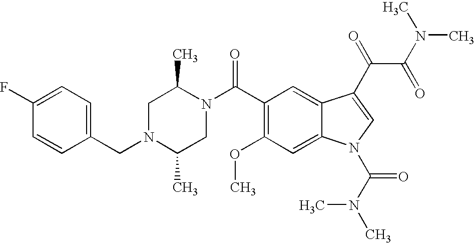 |
| 47 | 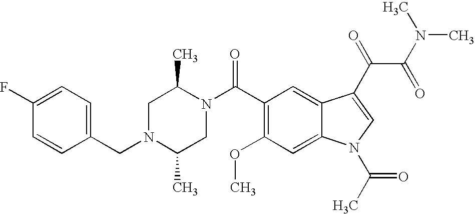 |
| 48 | 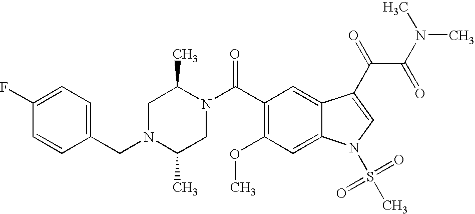 |
| 49 | 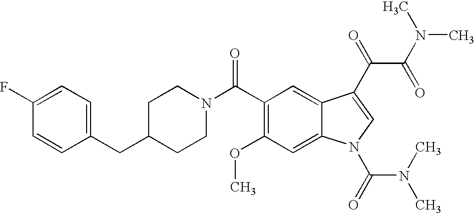 |
| 50 | 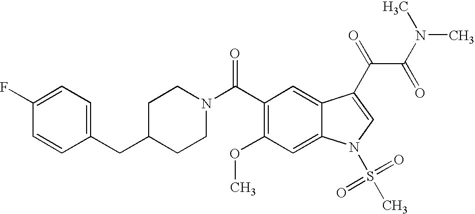 |
| 51 | 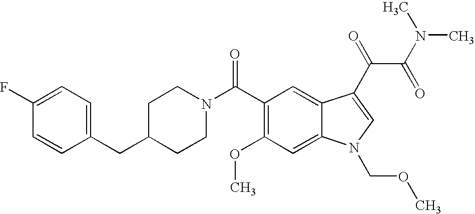 |
| 52 | 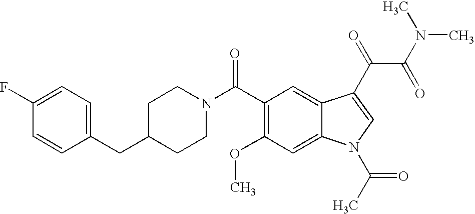 |
| 53 | 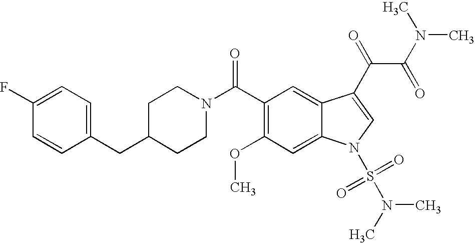 |
| 54 | 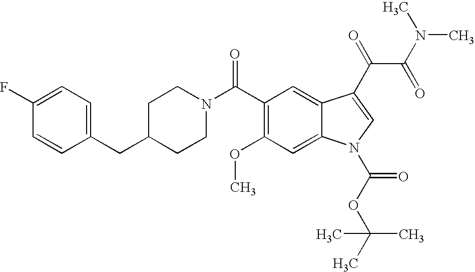 |
| 55 | 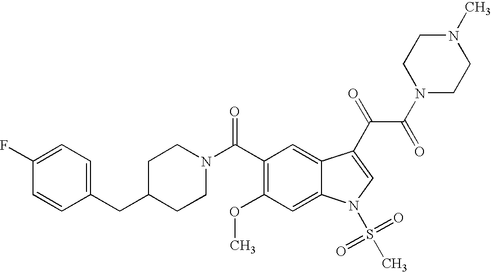 |
| 56 | 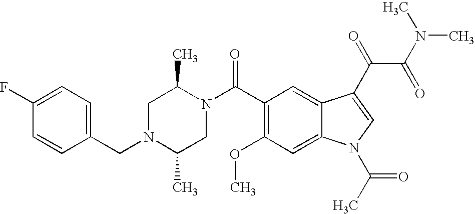 |
| 57 | 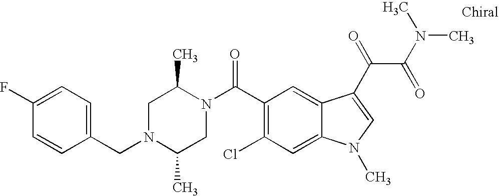 |
| 58 | 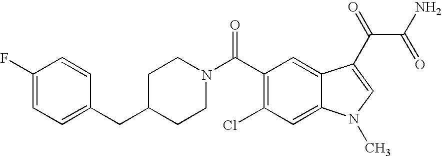 |
| 59 | 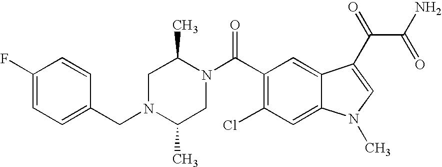 |
| 60 | 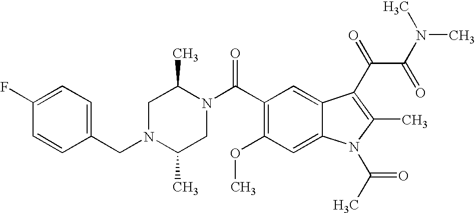 |
| 61 | 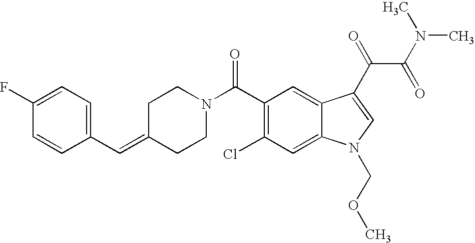 |
| 62 | 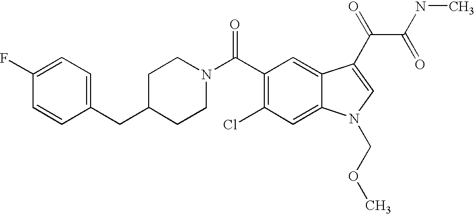 |
| 63 | 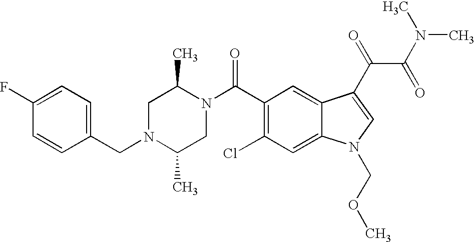 |
| 64 | 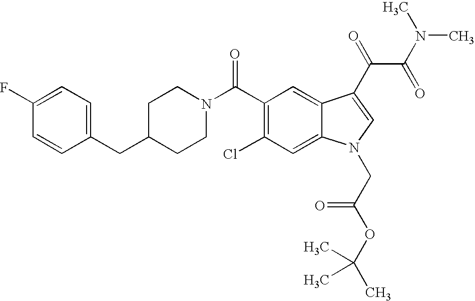 |
| 65 | 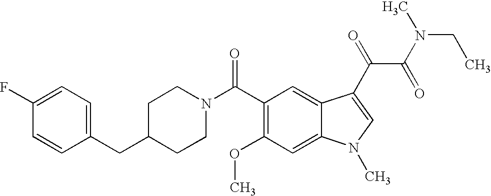 |
| 66 | 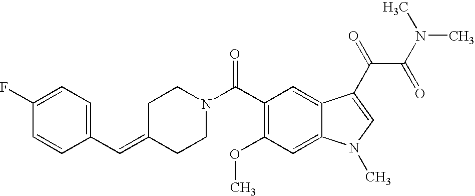 |
| 67 | 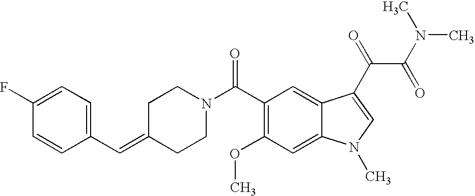 |
| 68 | 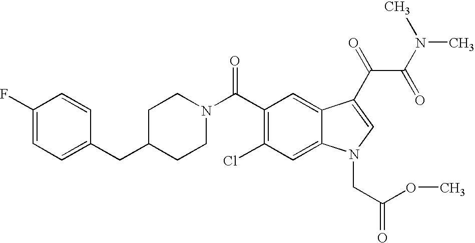 |
| 69 | 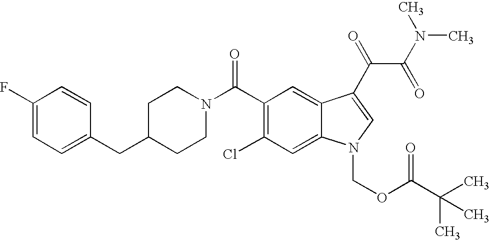 |
| 70 | 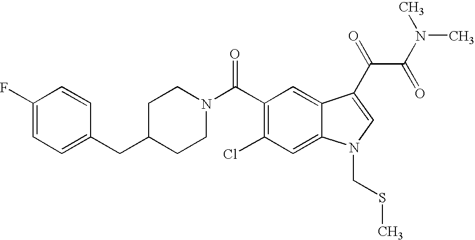 |
| 71 | 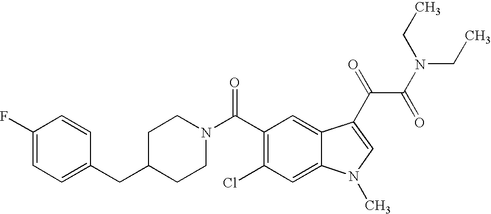 |
| 72 | 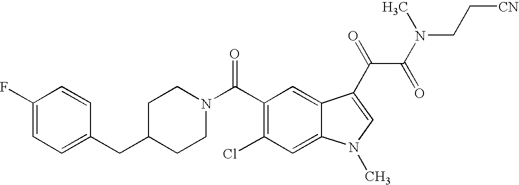 |
| 73 | 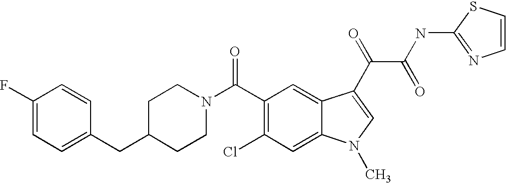 |
| 74 | 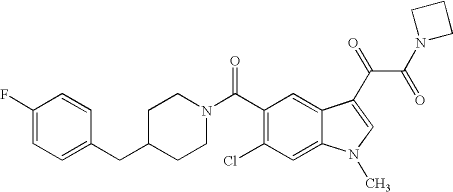 |
| 75 | 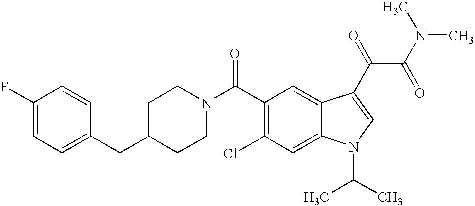 |
| 76 | 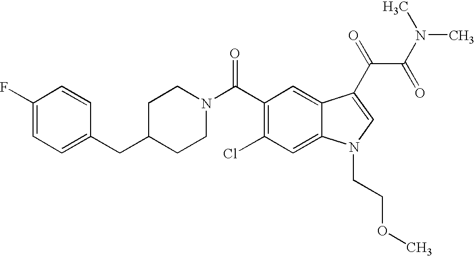 |
| 77 | 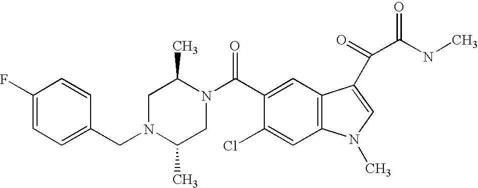 |
| 78 | 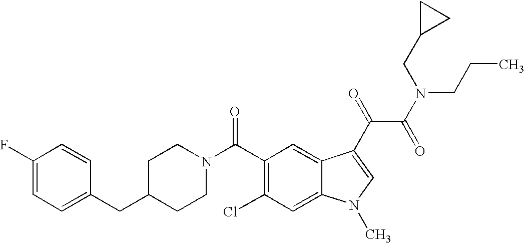 |
| 79 | 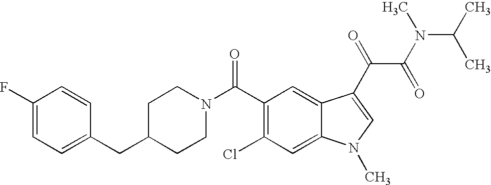 |
| 80 | 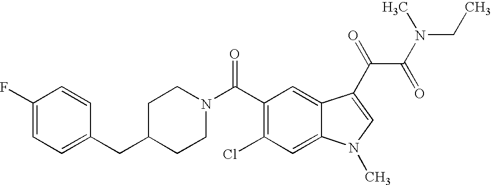 |
| 81 | 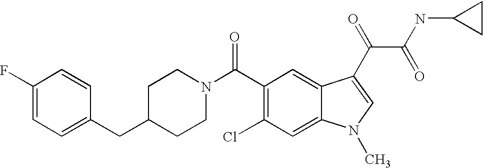 |
| 82 | 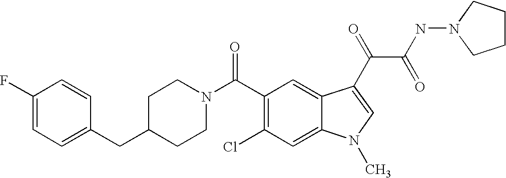 |
| 83 | 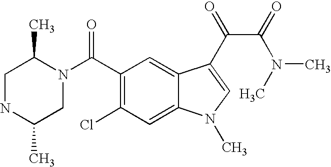 |
| 84 | 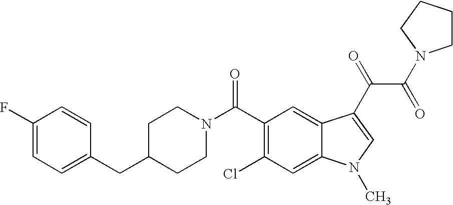 |
| 85 | 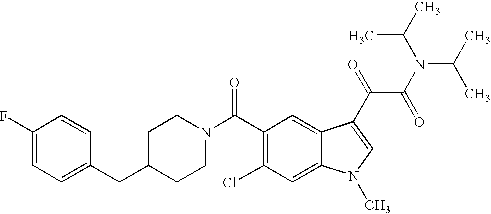 |
| 86 | 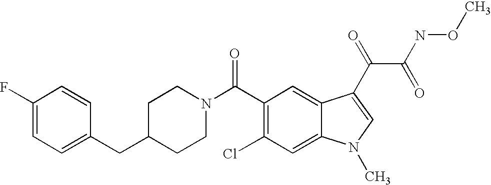 |
| 87 | 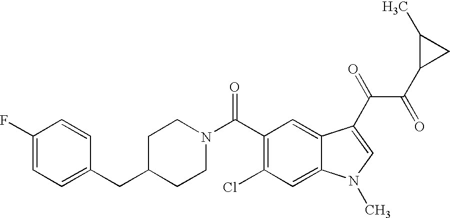 |
| 88 | 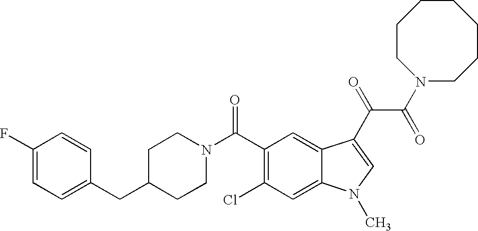 |
| 89 | 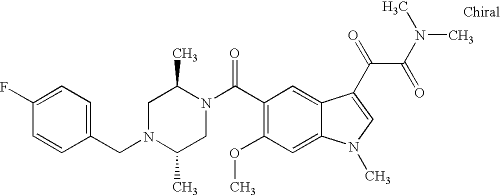 |
| 90 | 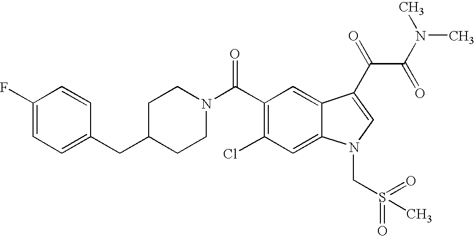 |
| 91 | 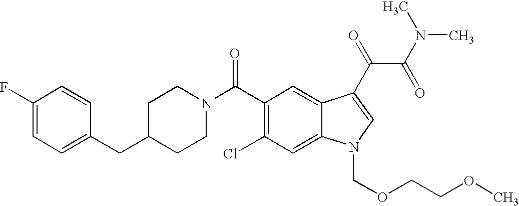 |
| 92 | 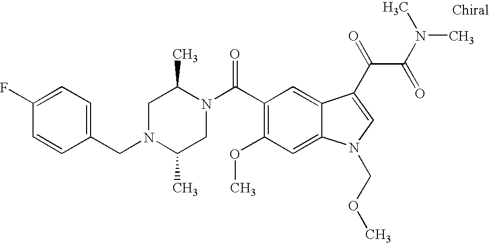 |
| 93 | 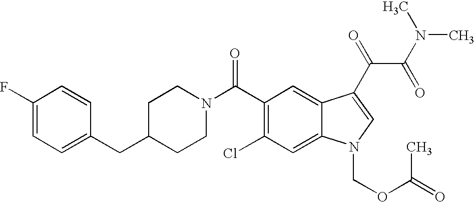 |
| 94 | 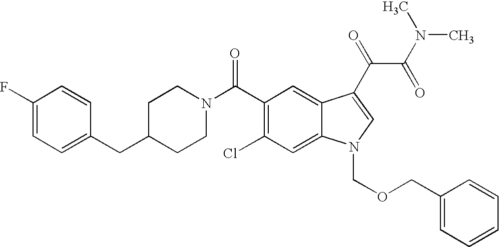 |
| 95 | 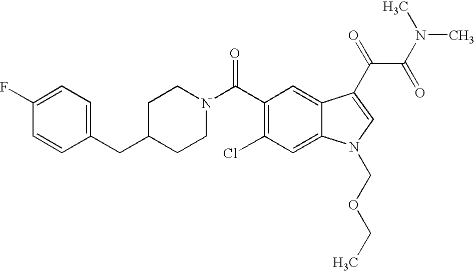 |
| 96 | 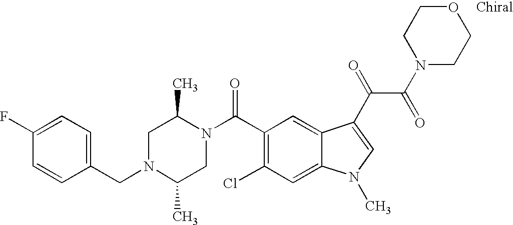 |
| 97 | 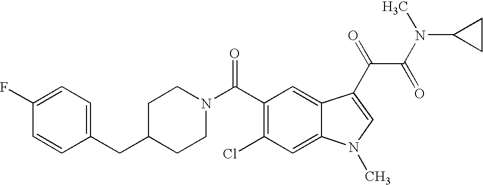 |
| 98 | 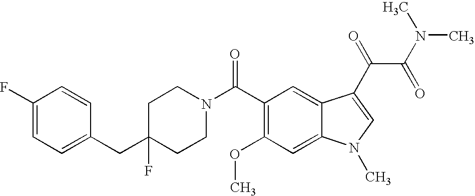 |
| 99 | 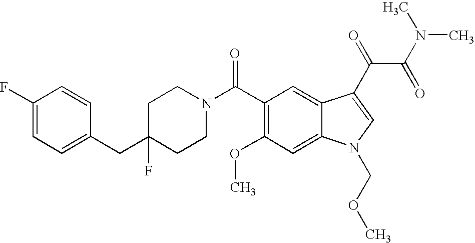 |
| 100 | 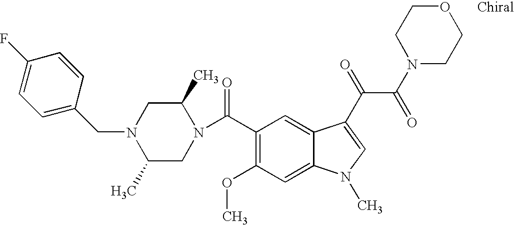 |
| 101 | 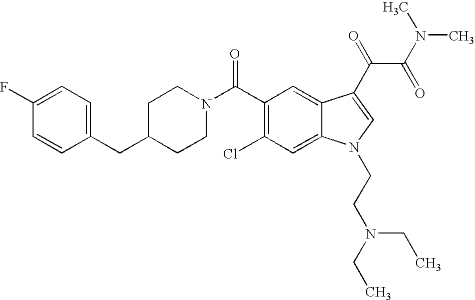 |
| 102 | 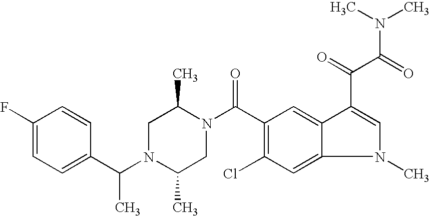 |
| 103 | 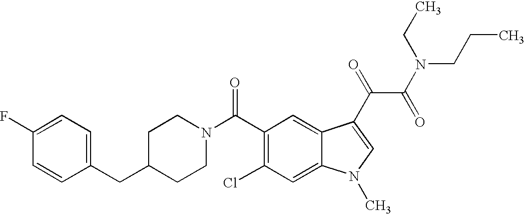 |
| 104 | 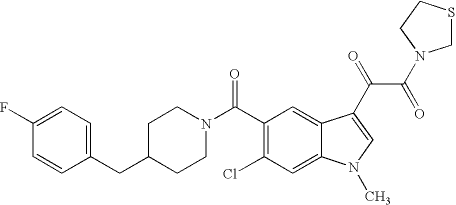 |
| 105 | 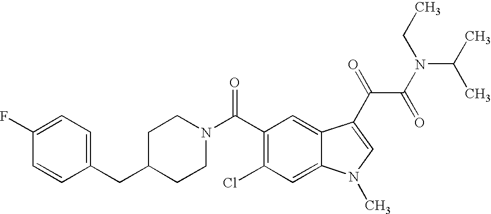 |
| 106 | 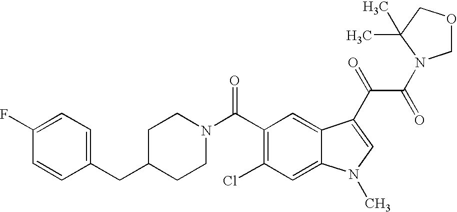 |
| 107 | 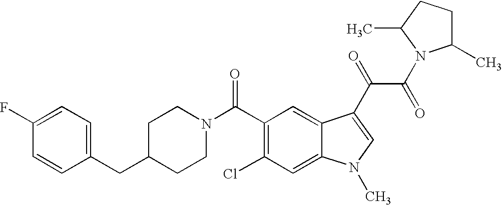 |
| 108 | 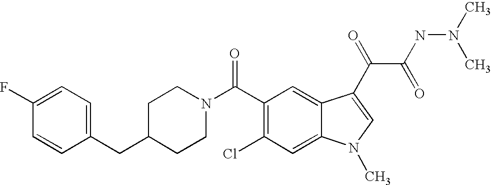 |
| 109 | 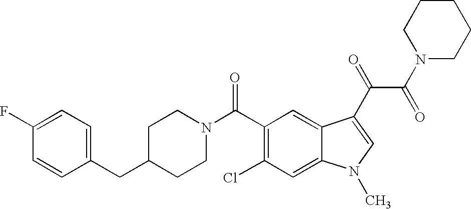 |
| 110 | 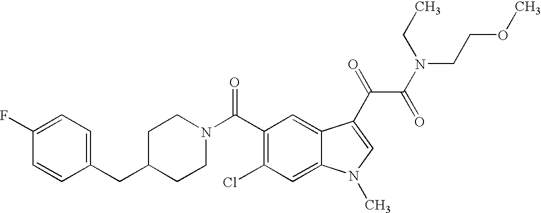 |
| 111 | 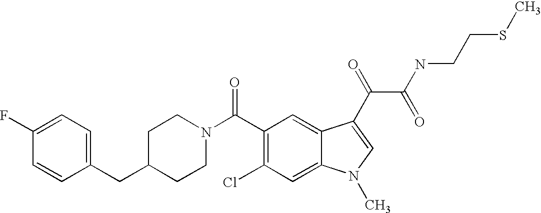 |
| 112 | 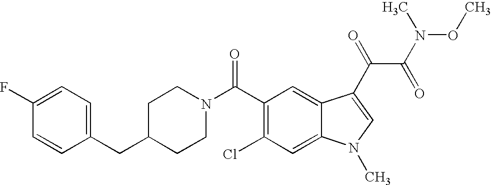 |
| 113 | 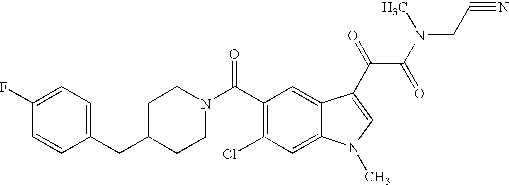 |
| 114 | 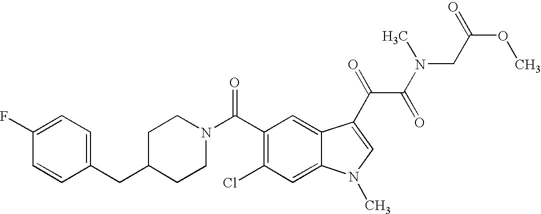 |
| 115 | 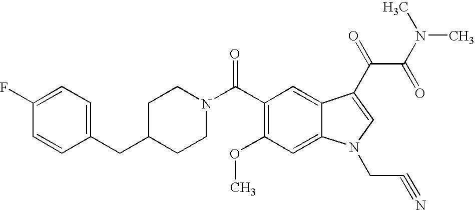 |
| 116 | 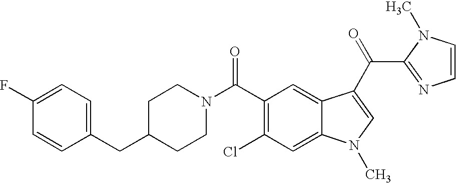 |
| Compd. # | MOLSTRUCTURE |
| 117 | 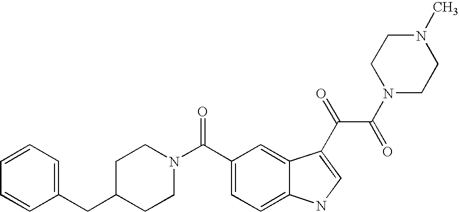 |
| 118 | 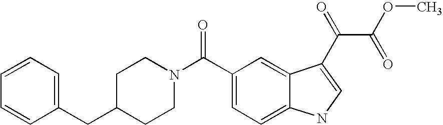 |
| 119 | 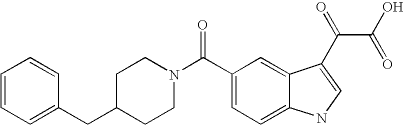 |
| 120 | 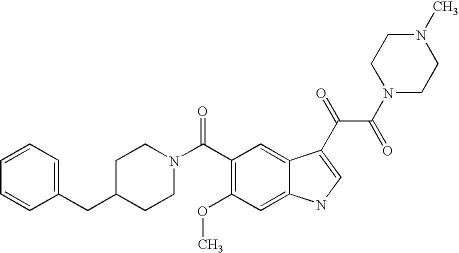 |
| 121 | 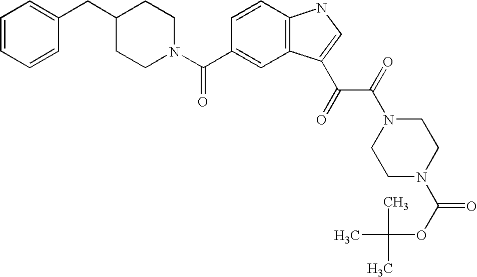 |
| 122 | 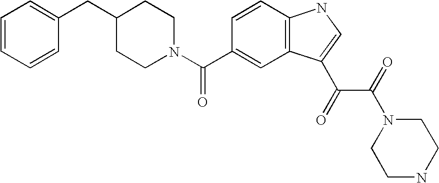 |
| 123 | 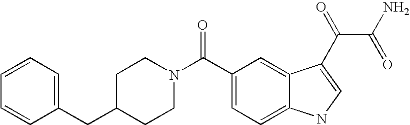 |
| 124 | 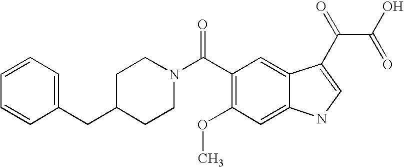 |
| 125 | 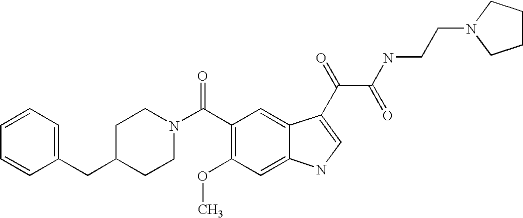 |
| 126 | 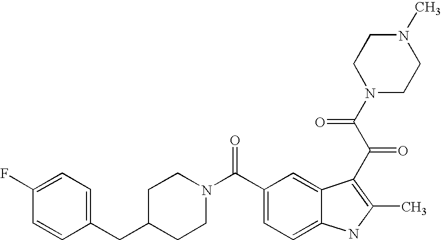 |
| 127 | 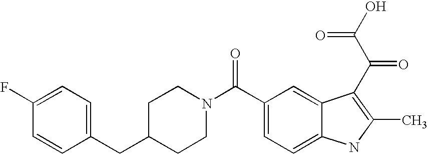 |
| 128 | 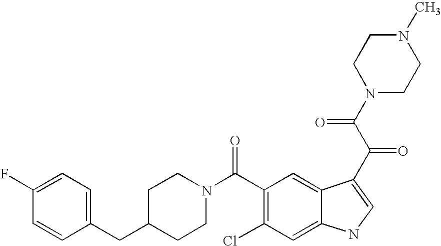 |
| 129 | 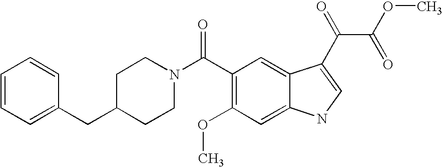 |
| 130 | 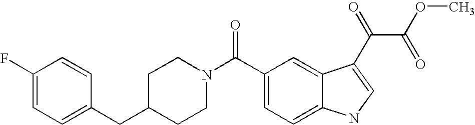 |
| 131 | 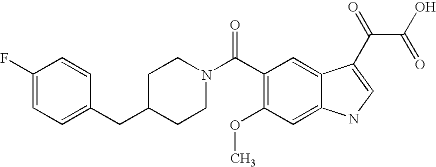 |
| 132 | 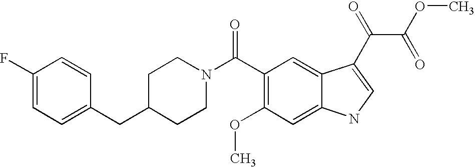 |
| 133 | 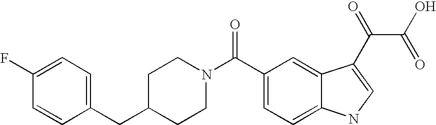 |
| 134 | 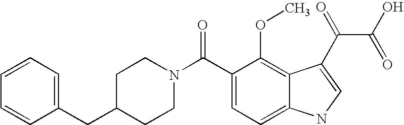 |
| 135 | 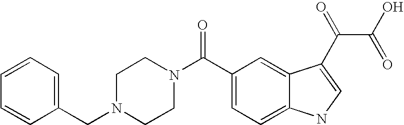 |
| 136 | 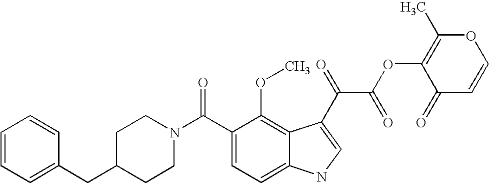 |
| 137 | 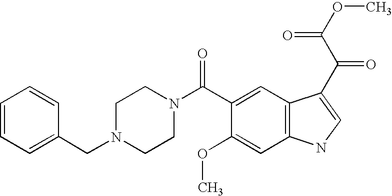 |
| 138 | 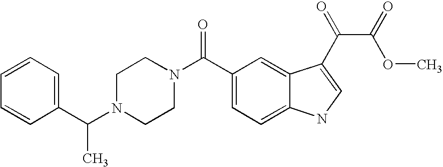 |
| 139 | 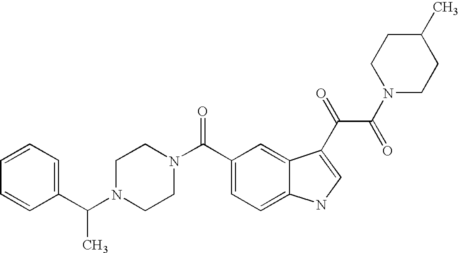 |
| 140 | 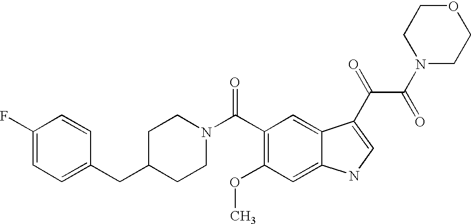 |
| 141 | 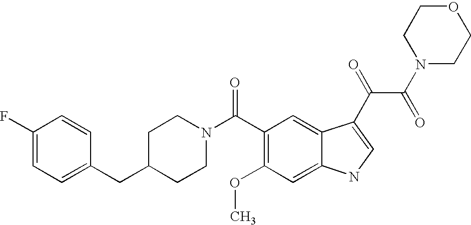 |
| 142 | 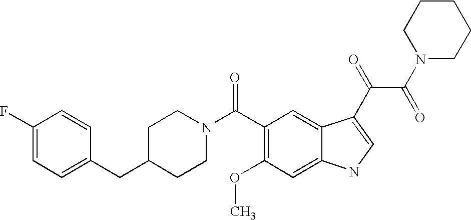 |
| 143 | 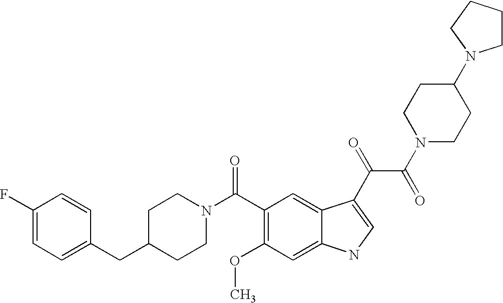 |
| 144 | 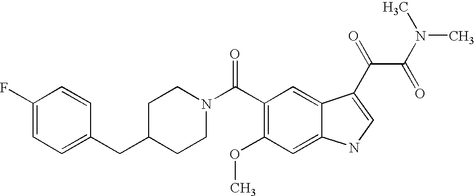 |
| 145 | 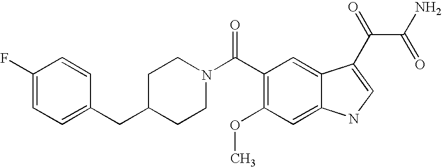 |
| 146 | 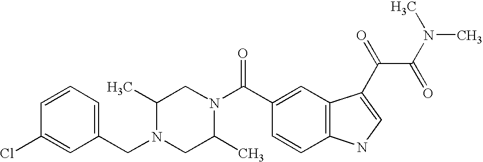 |
| 147 | 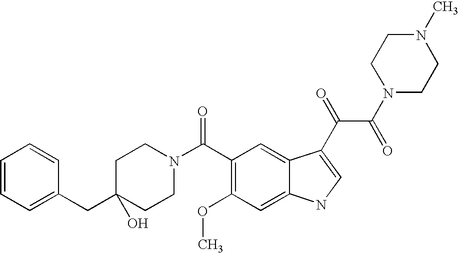 |
| 148 | 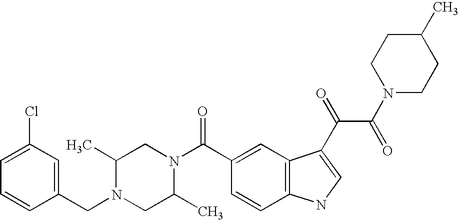 |
| 149 | 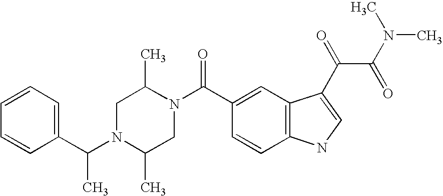 |
| 150 | 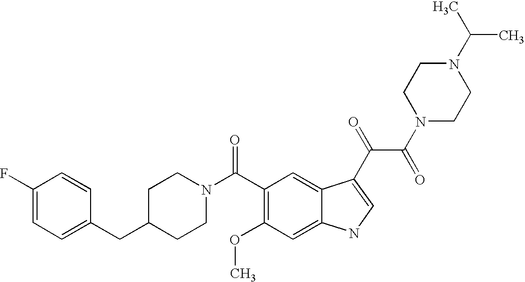 |
| 151 | 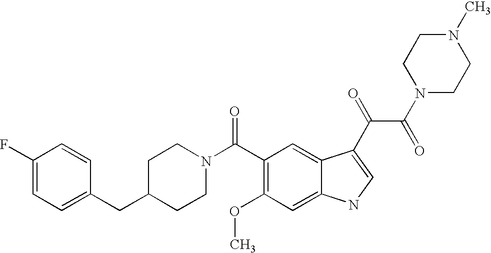 |
| 152 | 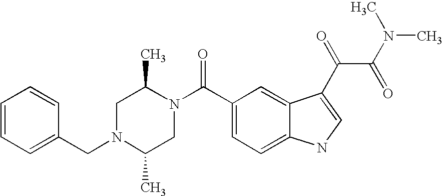 |
| 153 | 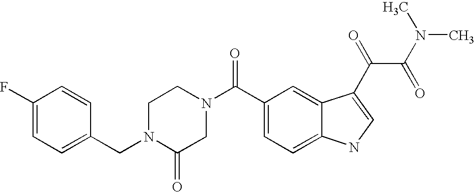 |
| 154 | 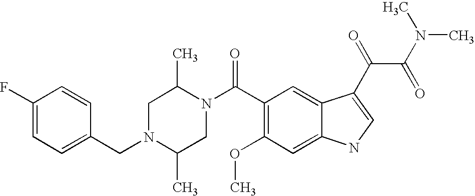 |
| 155 | 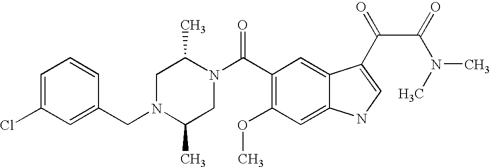 |
| 156 | 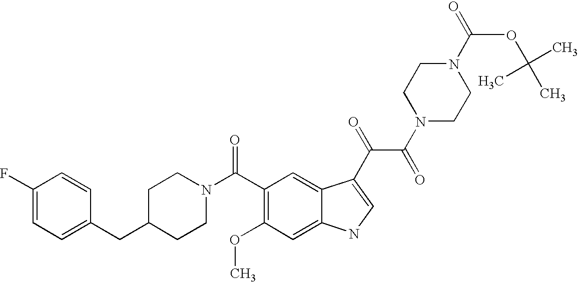 |
| 157 | 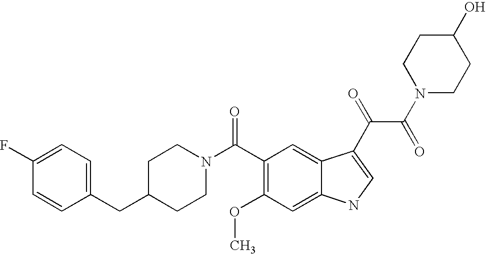 |
| 158 | 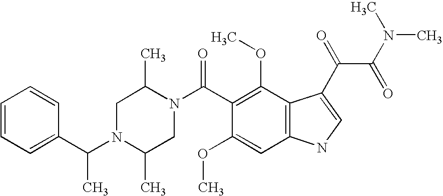 |
| 159 | 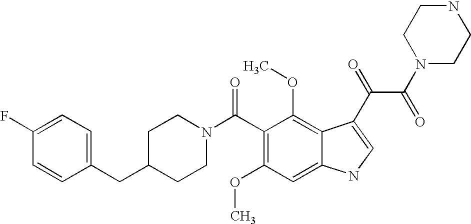 |
| 160 | 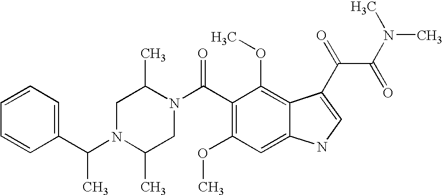 |
| 161 | 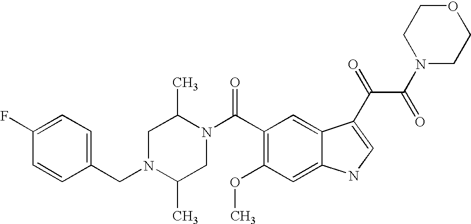 |
| 162 | 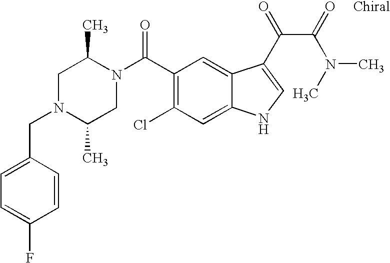 |
| 163 | 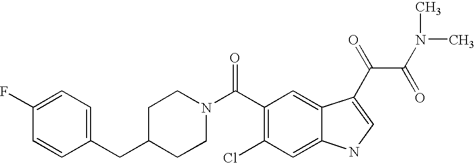 |
| 164 | 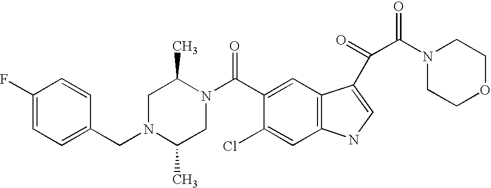 |
| 165 | 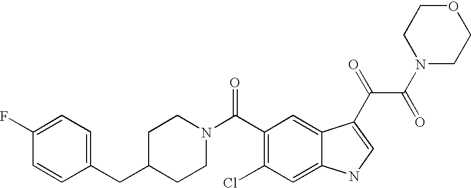 |
| 166 | 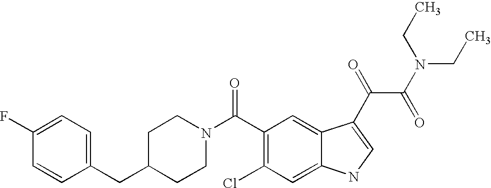 |
| 167 | 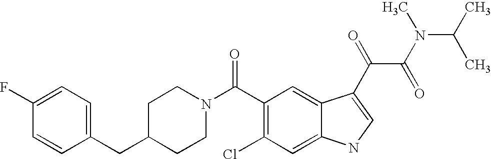 |
| 168 | 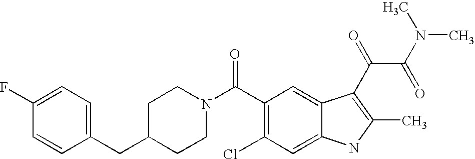 |
| 169 | 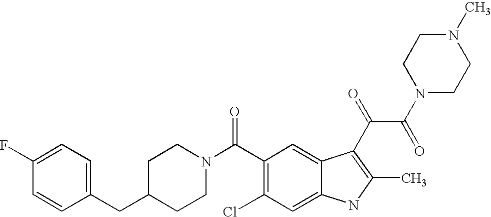 |

The compounds described above are provided for guidance and example only. It should be understood that other modulators of p38 kinase are useful in the invention provided that they exhibit adequate activity relative to the target protein.

Formulations and Methods of Administration

A pharmaceutical composition useful in the present invention comprises a p38 MAP kinase inhibitor (such as those described above) and a pharmaceutically acceptable carrier, excipient, diluent and/or salt.

Pharmaceutically acceptable carrier, diluent, excipient, and/or salt means that the carrier, diluent, excipient and/or salt must be compatible with the other ingredients of the formulation, does not adversely affect the therapeutic benefit of the p38 MAP kinase inhibitor, and is not deleterious to the recipient thereof.

Administration of the compounds or pharmaceutical compositions thereof for practicing the present invention can be by any method that delivers the compounds systemically and/or locally. These methods include oral routes, parenteral routes, intraduodenal routes, etc.

Depending on the particular condition, disorder or disease to be treated, additional therapeutic agents can be administered together with the p38 MAP kinase inhibitors. Those additional agents can be administered sequentially in any order, as part of a multiple dosage regimen, from the p38 MAP kinase inhibitor-containing composition (consecutive or intermittent administration). Alternatively, those agents can be part of a single dosage form, mixed together with the p38 MAP kinase inhibitor in a single composition (simultaneous or concurrent administration).

For oral administration, a pharmaceutical composition useful in the invention can take the form of solutions, suspensions, tablets, pills, capsules, powders, granules, semisolids, sustained release formulations, elixirs, aerosols, and the like. Tablets containing various excipients such as sodium citrate, calcium carbonate and calcium phosphate are employed along with various disintegrants such as starch, preferably potato or tapioca starch, and certain complex silicates, together with binding agents such as polyvinylpyrrolidone, sucrose, gelatin and acacia. Additionally, lubricating agents such as magnesium stearate, sodium lauryl sulfate and talc are often very useful for tabletting purposes. Solid compositions of a similar type are also employed as fillers in soft and hard-filled gelatin capsules; preferred materials in this connection also include lactose or milk sugar as well as high molecular weight polyethylene glycols. When aqueous suspensions and/or elixirs are desired for oral administration, the compounds of this invention can be combined with various sweetening agents, flavoring agents, coloring agents, emulsifying agents and/or suspending agents, as well as such diluents as water, ethanol, propylene glycol, glycerin and various like combinations thereof.

The choice of formulation depends on various factors such as the mode of drug administration (e.g., for oral administration, formulations in the form of tablets, pills or capsules are preferred) and the bioavailability of the drug substance. Recently, pharmaceutical formulations have been developed especially for drugs that show poor bioavailability based upon the principle that bioavailability can be increased by increasing the surface area i.e., decreasing particle size. For example, U.S. Pat. No. 4,107,288 describes a pharmaceutical formulation having particles in the size range from 10 to 1,000 nm in which the active material is supported on a crosslinked matrix of macromolecules. U.S. Pat. No. 5,145,684 describes the production of a pharmaceutical formulation in which the drug substance is pulverized to nanoparticles (average particle size of 400 nm) in the presence of a surface modifier and then dispersed in a liquid medium to give a pharmaceutical formulation that exhibits remarkably high bioavailability.

The term “parenteral” as used herein refers to modes of administration which include intravenous, intramuscular, intraperitoneal, intrastemal, subcutaneous, intramedullary and intraarticular injection and infusion. A pharmaceutical composition for parenteral injection can comprise pharmaceutically acceptable sterile aqueous or nonaqueous solutions, dispersions, suspensions or emulsions as well as sterile powders for reconstitution into sterile injectable solutions or dispersions just prior to use. Aqueous solutions are especially suitable for intravenous, intramuscular, subcutaneous and intraperitoneal injection purposes. In this connection, the sterile aqueous media employed are all readily obtainable by standard techniques well known to those skilled in the art. Examples of suitable aqueous and nonaqueous carriers, diluents, solvents or vehicles include water, ethanol, polyols (such as glycerol, propylene glycol, polyethylene glycol, and the like), carboxymethylcellulose and suitable mixtures thereof, vegetable oils (such as olive oil), and injectable organic esters such as ethyl oleate. Proper fluidity can be maintained, for example, by the use of coating materials such as lecithin, by the maintenance of the required particle size in the case of dispersions, and by the use of surfactants.

The pharmaceutical compositions useful in the present invention can also contain adjuvants such as, but not limited to, preservatives, wetting agents, emulsifying agents, and dispersing agents. Prevention of the action of microorganisms can be ensured by the inclusion of various antibacterial and antifungal agents, such as for example, paraben, chlorobutanol, phenol sorbic acid, and the like. It can also be desirable to include isotonic agents such as sugars, sodium chloride, and the like. Prolonged absorption of the injectable pharmaceutical form can be brought about by the inclusion of agents that delay absorption such as aluminum monostearate and gelatin.

In some cases, in order to prolong the effect of the drugs, it is desirable to slow the absorption from subcutaneous or intramuscular injection. This can be accomplished by the use of a liquid suspension of crystalline or amorphous material with poor water solubility. The rate of absorption of the drug then depends upon its rate of dissolution which, in turn, can depend upon crystal size and crystalline form. Alternatively, delayed absorption of a parenterally administered drug form is accomplished by dissolving or suspending the drug in an oil vehicle.

Injectable depot forms are made by forming microencapsule matrices of the drug in biodegradable polymers such as polylactide, polyglycolide, and polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissues.

The injectable formulations can be sterilized, for example, by filtration through a bacterial-retaining filter, or by incorporating sterilizing agents in the form of sterile solid compositions, which can be dissolved or dispersed in sterile water or other sterile injectable medium just prior to use.

Administration by slow infusion is particularly useful when intrathecal or epidural routes are employed. A number of implantable or body-mountable pumps useful in delivering compound at a regulated rate are known in the art. See, e.g., U.S. Pat. No. 4,619,652.

Suspensions, in addition to the active compounds, can contain suspending agents as, for example, ethoxylated isostearyl alcohols, polyoxyethylene sorbitol and sorbitan esters, microcrystalline cellulose, aluminum metahydroxide, bentonite, agar-agar, and tragacanth, and mixtures thereof.

The pharmaceutical compositions useful in the invention can also be administered by nasal aerosol or inhalation. Such compositions are prepared according to techniques well known in the art of pharmaceutical formulation and can be prepared as solutions in saline, employing benzyl alcohol or other suitable preservatives, absorption promoters to enhance bioavailability, fluorocarbons, and/or other conventional solubilizing or dispersing agents.

In nonpressurized powder compositions, the active ingredients in finely divided form can be used in admixture with a larger-sized pharmaceutically acceptable inert carrier comprising particles having a size, for example, of up to 100 μm in diameter. Suitable inert carriers include sugars such as lactose. Desirably, at least about 95% by weight of the particles of the active ingredient have an effective particle size in the range of about 0.01 to about 10 lm.

Alternatively, the composition can be pressurized and contain a compressed gas, such as, e.g., nitrogen, carbon dioxide or a liquefied gas propellant. The liquefied propellant medium and indeed the total composition are preferably such that the active ingredients do not dissolve therein to any substantial extent. The pressurized composition can also contain a surface active agent. The surface active agent can be a liquid or solid non-ionic surface active agent or can be a solid anionic surface active agent. It is preferred to use the solid anionic surface active agent in the form of a sodium salt.

The compositions useful in the present invention can also be administered in the form of liposomes. As is known in the art, liposomes are generally derived from phospholipids or other lipid substances. Liposomes are formed by mono- or multi-lamellar hydrated liquid crystals that are dispersed in an aqueous medium. Any non-toxic, physiologically acceptable and metabolizable lipid capable of forming liposomes can be used. The present compositions in liposome form can contain, in addition to the compounds of the invention, stabilizers, preservatives, excipients, and the like. The preferred lipids are the phospholipids and the phosphatidyl cholines (lecithins), both natural and synthetic. Methods to form liposomes are known in the art (see e.g., Prescott, E., Meth. Cell Biol. 14:33 (1976)).

Other pharmaceutically acceptable carrier includes, but is not limited to, a non-toxic solid, semisolid or liquid filler, diluent, encapsulating material or formulation auxiliary of any type, including but not limited to ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Solid pharmaceutical excipients include, but are not limited to, starch, cellulose, talc, glucose, lactose, sucrose, gelatin, malt, rice, flour, chalk, silica gel, magnesium stearate, sodium stearate, glycerol monostearate, sodium chloride, dried skim milk and the like. Liquid and semisolid excipients can be selected from glycerol, propylene glycol, water, ethanol and various oils, including those of petroleum, animal, vegetable or synthetic origin, e.g., peanut oil, soybean oil, mineral oil, sesame oil, etc. Preferred liquid carriers, particularly for injectable solutions, include water, saline, aqueous dextrose, and glycols.

Methods of preparing various pharmaceutical compositions with a certain amount of active ingredient are known, or will be apparent in light of this disclosure, to those skilled in this art. Other suitable pharmaceutical excipients and their formulations are described in Remington's Pharmaceutical Sciences, edited by E. W. Martin, Mack Publishing Company, 19th ed. (1995).

Pharmaceutical compositions useful in the present invention can contain 0.1%-95% of the compound(s) of this invention, preferably 1%-70%. In any event, the composition or formulation to be administered will contain a quantity of a compound(s) according to this invention in an amount effective to treat the condition, disorder or disease of the subject being treated.

One of ordinary skill in the art will appreciate that pharmaceutically effective amounts of the p38 MAP kinase inhibitor can be determined empirically and can be employed in pure form or, where such forms exist, in pharmaceutically acceptable salt, ester or prodrug form. The agents can be administered to a patient as pharmaceutical compositions in combination with one or more pharmaceutically acceptable excipients. It will be understood that, when administered to, for example, a human patient, the total daily usage of the agents or composition of the present invention will be decided within the scope of sound medical judgment by the attending physician. The specific therapeutically effective dose level for any particular patient will depend upon a variety of factors: the type and degree of the cellular response to be achieved; activity of the specific agent or composition employed; the specific agents or composition employed; the age, body weight, general health, sex and diet of the patient; the time of administration, route of administration, and rate of excretion of the agent; the duration of the treatment; drugs used in combination or coincidental with the specific agent; and like factors well known in the medical arts. For example, it is well within the skill of the art to start doses of the agents at levels lower than those required to achieve the desired therapeutic effect and to gradually increase the dosages until the desired effect is achieved.

Dosaging can also be arranged to provide a predetermined concentration of the compound in the blood of a patient, as determined by techniques accepted and routine in the art (HPLC is preferred). Depending on the particular compound to be used, one of ordinary skill can take into account, e.g., the IC₅₀ value and bioavailability of the compound to achieve the appropriate dosage. Determining dosages are within the purview of ordinary skill in the art.

Kits

The invention also relates to combining separate pharmaceutical compositions in kit form useful for treating diabetes. The kit can have a carrier means being compartmentalized in close confinement to receive two or more container means therein, having (1) a first container means containing a therapeutically effective amount of a p38 MAP kinase inhibitor and (2) a second container means containing a therapeutically effective amount of carrier, excipient or diluent. Optionally, the kit can have additional container mean(s) containing a therapeutically effective amount of additional agents.

The kit comprises a container for containing the separate compositions such as a divided bottle or a divided foil packet, however, the separate compositions can also be contained within a single, undivided container. Typically the kit comprises directions for administration of the separate components. The kit form is particularly advantageous when the separate components are preferably administered in different dosage forms (e.g., oral and parenteral) or at different dosage intervals, or when titration of the individual components of the combination is desired by the prescribing physician.

An example of such a kit is a so-called blister pack. Blister packs are well known in the packaging industry and are being widely used for the packaging of pharmaceutical unit dosage forms (tablets, capsules, and the like). Blister packs generally consist of a sheet of relatively stiff material covered with a foil of a preferably transparent plastic material. During the packaging process, recesses are formed in the plastic foil. The recesses have the size and shape of the tablets or capsules to be packed. Next, the tablets or capsules are placed in the recesses and the sheet of relatively stiff material is sealed against the plastic foil at the face of the foil which is opposite from the direction in which the recesses were formed. As a result, the tablets or capsules are sealed in the recesses between the plastic foil and the sheet. Preferably the strength of the sheet is such that the tablets or capsules can be removed from the blister pack by manually applying pressure on the recesses whereby an opening is formed in the sheet at the place of the recess. The tablet or capsule can then be removed via said opening.

It can be desirable to provide a memory aid on the kit, e.g., in the form of numbers next to the tablets or capsules whereby the numbers correspond with the days of the regimen which the dosage form so specified should be ingested. Another example of such a memory aid is a calendar printed on the card e.g., “First Week, Monday, Tuesday . . . Second Week, Monday, Tuesday.” etc. Other variations of memory aids will be readily apparent. A “daily dose” can be a single tablet or capsule or several tablets or capsules to be taken on a given day. Also, a daily dose of the compound, a prodrug thereof, or a pharmaceutically acceptable salt of the compound can consist of one tablet or capsule while a daily dose of the second compound can consist of several tablets or capsules and vice versa. The memory aid should reflect this.

It will be readily apparent to one of ordinary skill in the relevant arts that other suitable modifications and adaptations to the methods and applications described herein can be made without departing from the scope of the invention or any embodiment thereof.

The following examples are offered to illustrate but not to limit the invention.

### EXAMPLES

### EXAMPLES

In the following examples, compound 25 of Table B was used to evaluate the role of p38 inhibitors on the diabetes disease state. Other compounds such as compound 15, Table B, p38 MAPK modulator commercial available through Sigma-Aldrich® under product number S8307), compound 33, Table B, compound 183, Table B, compound 154, Table B, compound 2, Table B, compound 3, Table B, compound 84, Table B, compound 92, Table B, compound 96, Table B, compound 141, Table B, compound 169, Table B, and compound 67, Table B are all compounds that generally exhibit p38 MAPK activity with a relative IC₅₀ value of less than 5 nM, as observed in an assay similar to the phosphorylation assay disclosed above (see Kumar). These p38 MAPK inhibitors and others disclosed herein have utility in practicing the disclosed methods of treatment.

### Example 1

### Preventive and Therapeutic Studies

Female NOD mice were purchased from Jackson Labs, Bar Harbor, Me., USA and maintained in our animal facility. Mice were maintained in accordance with the guidelines of the Committee of Animals at Scios, Inc. All mice were kept under conventional conditions at a constant temperature (22-25° C.) and fed commercial powdered Purina chow diet and tap water ad libitum.

A p38 MAP kinase inhibitor, meeting a criteria of exhibiting an IC₅₀ value of about 5 μM or less, as quantitated according to Kumar, S. et al., Biochem. Biophys. Res. Commun. 235:533-538 (1997), was obtained from Scios, Inc., CA and used in the experiments described herein. While the present invention is not limited to compounds from Scios, Inc., other p38 MAP kinase inhibitors from Scios can be used, for example, compounds having an IC₅₀ value of about 5 μM to about 10 nM. In regard to dosaging, without being bound by the dosages provided herein as examples, a dosage of the compound that would result in a concentration of about 0.1 μM to about 10 μM in blood can be used. Preferably, a dosage of the compound that would result in a concentration of, e.g., about 0.2 μM to about 2 μM in blood can be used. More preferably, a dosage of the compound that would result in a concentration of, e.g., about 0.6 μM to about 1.8 μM in blood can be used.

### Preventive Studies

In the preventive studies, the animals were randomized into three groups. Group-1 (n=20) served as the vehicle (mice received powdered chow), Group-2 (n=20) received low dose of p38 MAP kinase inhibitor (a dose that gives 0.6 μM circulating concentration of p38 MAP kinase inhibitor in the blood), and Group-3 (n=20) received high dose of p38 MAP kinase inhibitor (a dose that gives 1.8 μM circulating concentration of p38 MAP kinase inhibitor in the blood), and were studied for a period of 10 weeks.

Food intake, body weight, development of diabetes, insulin and p38 MAP kinase inhibitor measurements: The food intake was inspected during the study to keep track of the dosing strengths of p38 MAP kinase inhibitor. Each mouse was weighed once a week. Development of diabetes was monitored by measuring blood glucose levels once a week. Blood glucose levels were measured by nicking the tail of the mouse and drawing a drop of blood onto glucose test strips (LifeScan One Touch Glucose Meter, Milpitas, Calif.). At the end of the study, each mouse was sacrificed by cervical dislocation. Terminal blood samples were assayed for serum levels of insulin using a mouse insulin ELISA kit (ALPCO Diagnostics, Windham, N. H.). p38 MAP kinase inhibitor in the serum samples were analyzed by LC/MS/MS (Applied Biosystems, CA) on a C, 18 column after protein precipitation with and without internal standard.

Histology: After cervical dislocation, the pancreas were fixed in 10% buffered formalin solution. Paraffin-embedded sections were stained by hematoxylin and eosin (H&E). The extent of lymphocytic infiltration (intensity of insulitis) in an islet was scored 0 to 4, with 0 indicating normal islet, 1 minimal, 2 mild, 3 moderate and 4 severe histological alterations.

Characterization of the infiltrate by immunohistochemistry: Using a different set of experimental mice, pancreatic tissues from 4, 8, 13 and 18 week-old NOD mice from vehicle and p38 MAP kinase inhibitor treated groups were embedded in Tissue-Tek O.C.T., frozen in a methylbutane tank, and stored at −70° C. Tissues were subjected to immunohistochemical staining for T cells (CD⁺5), CD4+, CD8+, microphages (Mac-3), TNF-α, TGF-β and p38 were performed. The primary antibodies used in this study were rat anti-mouse CD⁺5 (T cells) monoclonal antibody diluted at 1:25 (BD Pharmingen, Cat. # 55029); rat anti-mouse CD4⁺ (T helper cell) monoclonal antibody diluted at 1:25 (BD Pharmingen, Cat. # 550278); rat anti-mouse CD8⁺ (T suppressor/cytotoxic cells) monoclonal antibody diluted at 1:25 (BD PharMingen, Cat. # 550282); rat anti-mouse Mac-3 (macrophage) monoclonal antibody diluted at 1:25 (BD Pharmingen, Cat. # 550292); ratanti-mouse TNF-α monoclonal antibody diluted at 1:100 (BD Pharmingen, Cat. # 554416); goat TNF-α polyclonal antibody diluted at 1:50 (Santa Cruz, Cat. # Sc-¹³⁵I); rabbit TGF-β polyclonal antibody diluted at 1:50 (Santa Cruz, Cat. # S26-100 ml); rabbit p-p38 polyclonal antibody diluted at 1:50 (Santa Cruz, Cat. # Sc-7975-R). The secondary antibody used for Mac-3 was biotinylated mouse anti-rat IgG_{1/2a} diluted at 1:50 (BD Farmington, Cat. # 550325), and the negative control used was normal rat IgG₂ₐ. The secondary antibody used for TGF-β and p-p38 was goat anti-rabbit biotinylated IgG (Chemicon International, Inc., Cat. # AP 187B) diluted at 1:2000 and negative control used for them was normal rabbit IgG. The secondary antibody used for TNF-α was donkey anti-goat biotinylated IgG (Chemicon International, Inc., Cat. # AP 180B) diluted at 1:2000. After secondary treatment, all sections were treated with ABC reagents (Vector, Burlingame, Cat. # 6100) and finally stained with diaminobenzidine (Research Genetic, Cat. # 750118). Following treatment with secondary antibody, the sections were counterstained with hematoxylin and subsequently cover slipped with PERMOUNT mounting medium. The middle part of pancreas was evaluated, allowing examination of cross sections of at least 20 islets. Insulitis was evaluated by the total number of inflamed islets and the percentage of area in an islet which was infiltrated with T lymphocytes and scored as: 0 indicating normal islets; 1 indicating 1-5% of area of islet infiltrated by lymphocytes, 2 indicating 5-25% of the area of islet infiltrated by lymphocytes, 3 indicating 25-50% of the area of islet infiltrated by lymphocytes, and 4 indicating 50-75% of the area of islet infiltrated by lymphocytes. The total number of inflamed islets was also counted in each animal. p-p38 analysis was evaluated by the number of positively stained cells in an islet and scored as: 0 indicating no any positively stained cells in an islet, 1 indicating intense staining of 1-5 cells per islet, 2 indicating intense staining of 5-10 cells per islet, 3 indicating intense staining of 10-15 cells per islet, 4 indicating intense staining of more than 15 cells per islet. Tissue analysis was blinded.

### Therapeutic Studies

Studies on mildly hyperglycemic mice: Eighteen NOD mice having a blood glucose level of approximately 150 mg/dl (range of about 125 mg/dl to about 175 mg/dl) were randomized into three groups. Group-1 (n=7) served as the vehicle (mouse received powdered chow), Group-2 (n=7) received low dose of p38 MAP kinase inhibitor (a dose that gives 0.6 μM circulating concentration of p38 MAP kinase inhibitor in the blood), and Group-3 (n=7) received high dose of p38 MAP kinase inhibitor (a dose that gives 1.8 μM circulating concentration of p38 MAP kinase inhibitor in the blood) and studied for a period of 17 days. Each mouse was weighed once every three days. Blood glucose was measured once every three days by nicking the tail and drawing a drop of blood onto the glucose test strips (Life Scan One Touch Glucose Meter, Milpitas, Calif.). At the end of the study, each mouse was sacrificed by cervical dislocation. Terminal blood samples were assayed for serum levels of insulin using a mouse insulin ELISA kit (ALPCO Diagnostics, Windham, N. H.).

Fasting blood glucose and glucose tolerance studies in mildly hyperglycemic mice: In a different set of experiments, twelve NOD mice having blood glucose levels around 150 mg/dl were randomized into two groups. Group-1 (n=6) served as the vehicle (mice received powdered chow), and Group-2 (n=6) received high dose of p38 MAP kinase inhibitor (a dose that gives 1.8 μM circulating concentration of p38 MAP kinase inhibitor in the blood), and were studied for a period of 17 days. Fasting blood glucose levels were measured on overnight fasted mice on day 17. Glucose tolerance test was conducted by fasting the mice overnight on day 17; oral administering of glucose at a dose of 2 g/kg of body weight; and measuring blood glucose at 0 (the values before glucose challenge), 30, 60, 120 minutes after glucose challenge.

Studies on severely hyperglycemic mice: Twelve NOD mice showing blood glucose of approximately 450 mg/dl (range of about 400 mg/dl to about 500 mg/dl) were randomized into two groups. Group-1 (n=6) served as the control (mouse received powdered diet), and Group-2 (n=6) received high dose of p38 MAP kinase inhibitor (a dose that gives 1.8 μM circulating concentration of p38 MAP kinase inhibitor in the blood) and studied for a period of 17 days. Each mouse was weighed once every three days. Blood glucose was also measured once every three days by nicking the tail and drawing a drop of blood onto glucose test strips (Life Scan One Touch Glucose Meter, Milpitas, Calif.).

Statistic analysis: Data compared between treatments was analyzed for significance with one-way ANOVA followed by a post-hoc Bonferroni analysis in most of the cases. Two-tailed unpaired test was used to determine a significant difference between the two groups. In studies on blood glucose levels in mildly hyperglycemic mice, the development of a hyperglycemia state was evaluated separately in each group by comparing each time point to the value obtained at 0, 3, 7, 10, 14 and 17 days by repeated measures analysis of variance with Dunnett multiple comparisons post test (<0.05 is considered significant). All analyses were performed using InStat (GraphPad, version 3.0).

### Results

### Preventive Studies

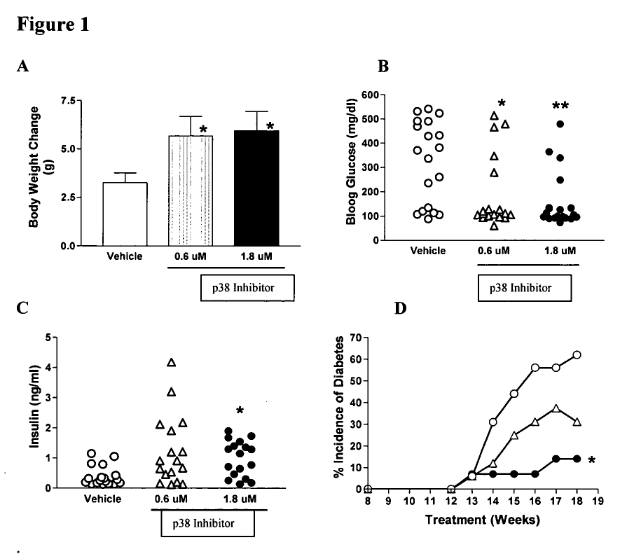

Food intake, serum p38 MAP kinase inhibitor, body weight, blood glucose, serum insulin and development of diabetes: Pre-diabetic NOD mice given p38 MAP kinase inhibitor in their diet consumed the same diet as that of the vehicle group. Analysis of p38 MAP kinase inhibitor in the serum showed 0.6 μM in low dose group and 1.8 μM in high dose group during the study period. Body weight, blood glucose and serum insulin levels are shown in FIGS. 1A-1C. NOD mice given p38 MAP kinase inhibitor significantly reduced body weight loss when compared to the vehicle treated group (FIG. 1A). p38 MAP kinase inhibitor at both dose groups significantly lowered blood glucose levels (FIG. 1B). p38 MAP kinase inhibitor at both dose groups increased serum insulin levels and the increase at high dose is statistically significant (FIG. 1C). There was a statistically significant (*p<0.01 vs. vehicle group) and dose-dependent delay in the onset of diabetes as defined by blood glucose levels greater than 120 mg/dl (FIG. 1D). Diabetes incidence at 18 weeks was 60% in vehicle-treated mice, 30% in the mice treated with the p38 MAP kinase inhibitor at low dose and 10% in the mice treated with the p38 MAP kinase inhibitor at high dose. The high dose of p38 MAP kinase inhibitor almost prevented the development/incidence of diabetes.

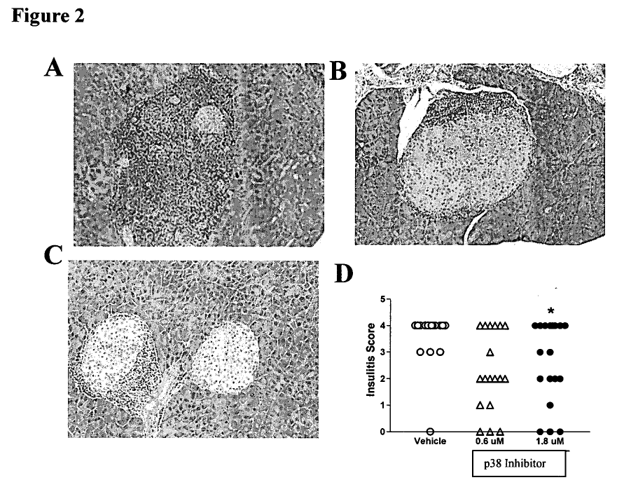

Histology: Pancreata of p38 MAP kinase inhibitor and vehicle treated mice were histologically (H&E) examined after 10 weeks of treatment. The pancreata of NOD mice from vehicle group showed destruction of islets of Langerhans with a severe lymphocytic infiltration (FIG. 2A). In contrast, the pancreata of mice treated with p38 MAP kinase inhibitor both at low and high doses showed only minor lymphocyte infiltration (FIGS. 2B, 2C). Quantitative histological assessment showed that p38 MAP kinase inhibitor treatment at both doses significantly (*p<0.05 vs. vehicle group) suppressed insulitis scores (FIG. 2D).

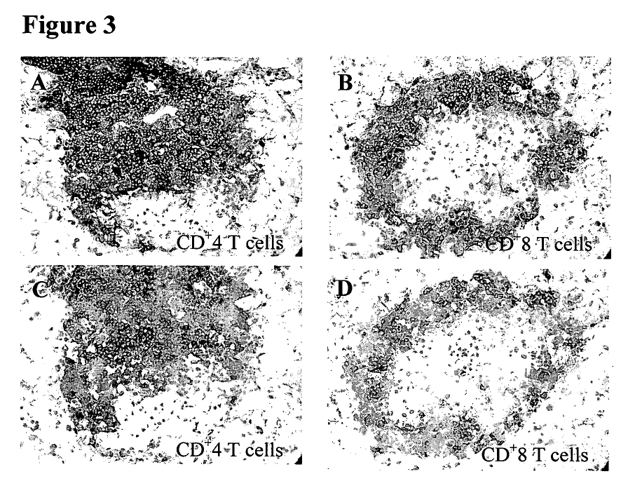

Characterization of the infiltrate by immunohistochemistry: Type of infiltrated T cells into the beta cell mass, severity of insulitis, area of beta cell mass affected by T cells, and expression of p38 were characterized by immunohistochemical analysis. The pancreata of NOD mice from vehicle-treated group showed severe insulitis with CD⁺5 T cells which were 90% of the total lymphocytes that infiltrated the beta cells. CD⁺4 (FIGS. 3A, 3C) and CD⁺8 (FIGS. 3B, 3D) T cells in the islets of vehicle (FIGS. 3A, 3B) and p38 MAP kinase inhibitor (FIGS. 3C, 3D) treated NOD mice were immunohistochemically stained. In mice treated with and without p38 MAP kinase inhibitor for 10 weeks, 90% of the infiltrating lymphocytes were shown by immunohistochemistry to be CD⁺5 T cells. 80% of the infiltrating T cells were CD⁺4 and 20% were CD⁺8. Treatment with p38 MAP kinase inhibitor at high dose remarkably suppressed CD⁺4 (FIG. 3C) and CD⁺8 (FIG. 3D) T cells infiltration into the beta cells without affecting their ratio.

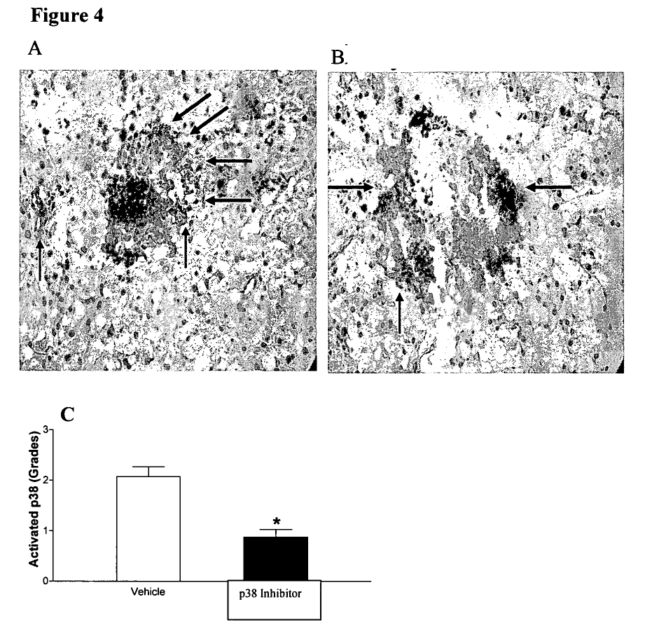

p38 expression in the T cells infiltrated into the beta cells of vehicle (FIG. 4A) and p38 MAP kinase inhibitor (FIG. 4B) treated NOD mice was observed. After 10 weeks of treatment, enhanced p38 MAP kinase expression was observed (see arrows) both in cytoplasm and nucleus of the T cells infiltrated into the beta cell mass of the vehicle treated group (FIG. 4A). In contrast, p38 MAP kinase inhibitor significantly reduced p38 MAP kinase expression in the T cells. Summarized results on p38 expression are shown as grades (FIG. 4C). Treatment with p38 MAP kinase inhibitor significantly decreased the p38 expression in the T cells (*p<0.001 vs. vehicle group). Macrophages (Mac-3) were not observed among these infiltrating cells at 18 weeks. TNF-α and TGF-β were also not detected in Islets of Langerhans.

### Therapeutic Studies

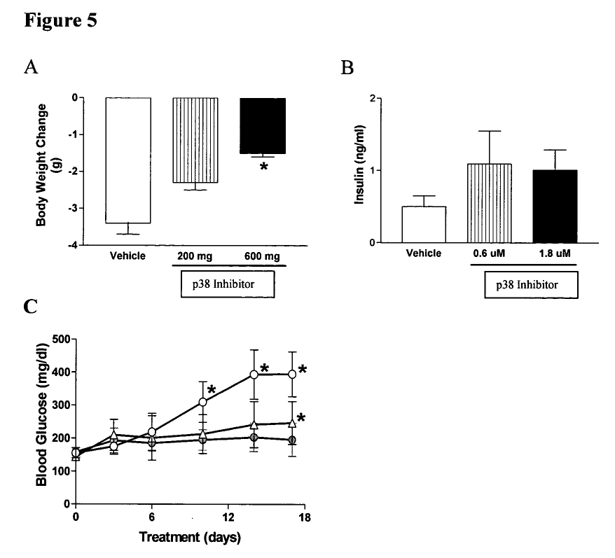

Studies on mildly hyperglycemic mice: Therapeutic effects of p38 MAP kinase inhibitor on blood glucose levels in mildly hyperglycemic NOD mice were observed. Mildly hyperglycemic NOD mice treated with p38 MAP kinase inhibitor for 17 days had decrease in weight loss (*p<0.05 vs. vehicle group) (FIG. 5A); and higher insulin levels (FIG. 5B) when compared to the vehicle treated group. In vehicle-treated NOD mice, (severe) hyperglycemia developed significantly by day-17 when compared to its baseline value. Whereas, p38 MAP kinase inhibitor dose-dependently prevented the development of hyperglycemia and the mice are mildly hyperglycemic by day-17 (FIG. 5C).

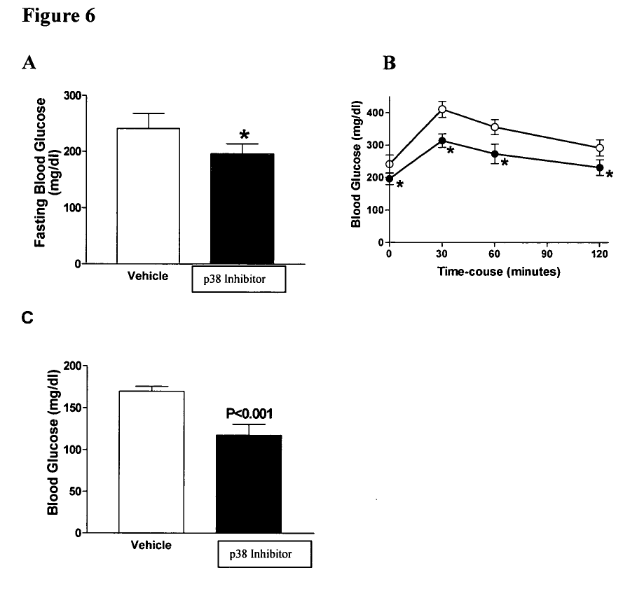

Fasting blood glucose and glucose tolerance studies in mildly hyperglycemic mice: Therapeutic effects of p38 MAP kinase inhibitor on blood glucose levels in mildly hyperglycemic NOD mice were observed. Mildly hyperglycemic NOD mice treated with high dose of p38 MAP kinase inhibitor for 17 days had lower fasting blood glucose levels (*p<0.05 vs. vehicle group) (FIG. 6A) when compared to the vehicle treated group. Oral glucose tolerance was evaluated on day 17 following an overnight fast. Blood glucose was measured immediately prior to and 30, 60, and 120 minutes following an oral glucose challenge (2 g/kg). p38 MAP kinase inhibitor for 17 days had improved glucose tolerance (*p<0.05 vs. vehicle group) when compared to the vehicle group (FIG. 6B). p38 MAP kinase inhibitor at high dose showed highly significant improvement in glucose tolerance at 30 minutes of the test (*p<0.001 vs. vehicle group) (FIG. 6C).

Studies on severely diabetic mice: p38 MAP kinase inhibitor had no effect on body weight, blood glucose and serum insulin levels in severely hyperglycemic NOD mice at high dose.

### Discussion

Unlike the earlier compounds which suffer from undesirable liver effects, current p38 MAP kinase inhibitors have much reduced effects on cytochrome P450 (Adams, J. L. et al., Bioorg. Med. Chem. Lett. 8:3111-3116 (1998)). Several of these newer generation inhibitors are currently being evaluated in clinical trails for leukocyte driven inflammatory diseases (Herlaar, E. and Brown, Z., Molec. Med. Today 5:439-447 (1999)). Type 1 diabetes is a T cell driven autoimmune disorder characterized by a local inflammatory reaction in and around pancreatic beta cell mass (Yoshida, K. and Kikutani, H., Rev. Immunogenetics 2:140-146 (2000)). Now that subjects at high risk for Type 1 diabetes can be identified (Ryu, S. et al., J. Clin. Invest. 108:63-72 (2001); Mahon, J. L. et al., Ann. N.Y. Acad. Sci. 696:351-363 (1993); Shapiro, A. M. et al., Diabetologia 45:224-230 (2002)), a major goal is to reduce the incidence of diabetes by disease-specific nontoxic agents such as p38 MAP kinase inhibitors.

In this study, it has been shown that p38 MAP kinase inhibitor prevents the development of diabetes in NOD mouse, a model of human Type 1 diabetes. The mode of p38 MAP kinase inhibitor action in vivo in preventing the development of diabetes in NOD mice involves the removal of T cells from beta cells, suppression of insulitis, preservation of insulin producing beta cells, elevation of insulin levels in the blood, reduction of blood glucose levels, and inhibition of body weight loss. However, the key in vivo action of p38 MAP kinase inhibitor is termination of insulitis by removing the T cells from the pancreatic beta cell mass (FIGS. 2A-2D).

Most of the T lymphocytes (CD⁺5) that p38 MAP kinase inhibitor removed are CD⁺4 cells which are 80% of the total T cells that infiltrated the beta cells and only 20% are CD⁺8 cells. p38 MAP kinase inhibitor did not change the ratio of the CD⁺4 and CD⁺8 T cells. The mechanism by which p38 MAP kinase inhibitor removes the T cells is unclear at present. Historical data suggests that a block in the activation of PKC/Ras/MAPK pathway mediates hypo-responsiveness of T cells in NOD mice (Zhang, J. et al., International Immunology 13:377-384 (2001); Rapport, M. J. et al., J. Exp. Med. 177:1221-1227 (1993)), but the kind of block that p38 MAP kinase inhibitor imposes on p38 MAP kinase signaling pathway needs to be elucidated. The potential physiological target for p38 MAP kinase is monocyte chemoattractant protein (MCP-1) (Goebeler, M. et al., Blood 93:857-865 (1999)). p38 signaling pathway attracts monocytes and T cells, and it could contribute to T cell infiltration into beta cells (Chen, M. C. et al., Diabetologia 44:325-332 (2001)). Interestingly, there is a parallelism between T cell infiltration and MCP-1 mRNA expression in islets from NOD mice and this parallelism reaches to a peak level by 8 weeks (Burysek, L. et al., J. Biol. Chem. 277:33509-33517 (2002)). This is well supported by SB203580 blockade of monocyte migration (Chen, M. C. et al., Diabetologia 44:325-332 (2001)), and p38 and ERK ½ inhibitors inhibition of MCP-1 expression in rat beta cells (Burysek, L. et al., J. Biol. Chem. 277:33509-33517 (2002)). Further, perforin, a cytolitic protein which is secreted by CD⁺8 T cells, remains one of the only molecules confirmed to be implicated in beta cell death in the NOD mouse (Thomas, H. E. and Kay, T. W. H., Diabetes/Metabolism Res. Rev. 16:251-261 (2000); Ravelli, A., Curr. Opin. Rheumatol. 14:548-552 (2002)). Perforin controls T cell proliferation and its deficiency dramatically reduces the development of diabetes in NOD mice (Balasa, B. et al., J Immunology 165:2841-2849 (2000)).

p38 is not expressed in the T cells of the beta cell mass of the pre-diabetic (4 and 8 weeks) NOD mice. It is expressed lightly in 13 week- and heavily in 18 week-old hyperglycemic NOD mice (FIG. 5). p38 MAP kinase inhibitor lowered p38 expression in the T cells of the beta cell mass of 18 week-old hyperglycemic NOD mice. Devoid of p38 expression in the pre-diabetic period and heavy expression in hyperglycemic diabetic period may be due to the alterations in islets of Langerhans cell biology induced by elevated blood glucose levels (Cohen, M. P., “Diabetes and Protein Glycation” in: Clinical and Pathophysiologic Relevance, JC Press, Philadelphia, Pa. (1996)). Activation of the p38 MAP kinase pathway has been implicated with some of the adverse complications such as advanced glycation end products associated with hyperglycemia (Blair, A. et al., J. Biol. Sci. 17:36293-36299 (1999)). In fact, glucose potentiates IL-1 beta induced p38 MAP kinase activity in rat pancreatic beta cells (Sprinkel, A. M. et al., Eur. Cytokine Netw. 12:331-339 (2001); Pavlovic D. et al., Eur. Cytokine Netw. 11:267-274 (2000)). The intriguing observation in this study is why the p38 is expressed only in the T cells of the beta cell mass and not in other cells of pancreas? This may be partly due to T cell responsiveness mediated signaling along the PKC/Ras/p38 MAP kinase pathway of T cell activation. Block in this activation mediates hypo-responsiveness in NOD T cells (Salomon, B. et al., Immunity 12:431-437 (2000); Rapport, M. J. et al., J. Exp. Med. 177:1221-1227 (1993)).

The immunosuppressive agent cyclosporine removes T cell infiltration into the beta cells and prevents the development of diabetes in NOD mice (Mori, Y. et al., Diabetologia 29:244-247 (1986)). However, it has no glucose lowering effect in mildly hyperglycemic NOD mice whereas p38 MAP kinase inhibitor significantly alleviates hyperglycemia in mildly hyperglycemic NOD mice. This means p38 MAP kinase inhibitor improved glucose homeostasis by recovering some beta cell mass in the damaged pancreas which has been shown to coincide with improved tolerance (i.e., return of insulin release) in response to a glucose load (FIG. 6). These observations strongly suggest p38 MAP kinase inhibitor preserves pancreatic beta cell mass in NOD mice. These results indicate that p38 MAP kinase inhibitor has therapeutic effects on diabetes in NOD mice, and suggests that it can reverse the beta cell damage at a very early state of Type 1 diabetes. The honeymoon period in Type 1 diabetes children is characterized by the preserved beta cell function. Therefore, interruption of the ongoing self-destruction of the remaining beta cells by a p38 inhibitor seems to be a viable approach. It is also presumed that early aggressive control of blood glucose levels with p38 inhibitor should remediate the relative beta cell exhaustion and allow for short-term glucose homeostasis without exogenous insulin. Since serum auto-antibodies can be detected while islet cells are being destroyed, intervention with a p38 inhibitor is expected to help during the onset of the disease in children at high risk as well as in children in the honeymoon period.

### Example 2

### p38 MAP Kinase Inhibitor Prevents Incidence of Diabetes in NOD Mice: Possibly via HSP 60

Type-1 diabetes in humans is the result of selective autoimmune attacks against pancreatic islet beta cells. The NOD mice are a species, which develops spontaneously autoimmune beta cell destruction similar to type-i diabetes in humans. NOD mice develop inflammatory insulitis between 8 to 13 weeks and overt diabetes after 18 weeks. During the pre-diabetic phase different protective and/or repair mechanisms might be activated in beta cells. It has been suggested that heat shock proteins (hsp) are involved in the islet cell repair mechanism during beta cell destruction process. In addition to hsp60, several other autoantigens have been reported to be involved in human and mouse type-I diabetes. Interestingly, the available literature indicates that hsp 60 plays a significant role in NOD mice. For example, HSP antigens and anti-HSP65 antibodies have been reported as being up-regulated in the pre-diabetic phase of the NOD mice. These immune markers decline in these mice with the development of overt insulin dependent diabetes.

It has also been demonstrated that the onset of diabetes is preceded by an increase in T cell reactivity toward HSP60 and to an HSP60 peptide contained between amino acids 437 and 460 named p277. Intraperitoneal treatment of NOD mice with a peptide p277 prevents diabetes. It has been also shown that HSP 70 can protect beta cells against the deleterious effects of IL-1 beta. Available literature also indicates that hsp70 prevents activation of various stress kinases. Bellman and his colleagues demonstrated p38-dependent enhancement of cytokines expression by hsp70 and p38 inhibitor SB203580 blocks this activity in rat insulinoma cells. Another study showed evidence on hsp70-mediated enhancement of the activation of p38. It has also been shown that activation of MAP kinases and HSP25 contributes to IL-1 beta induced cell death in purified rat pancreatic beta cells. More recent evidence has suggested increased p38 MAP kinase expression was observed both in cytoplasm and nucleus of the T cells infiltrated into the pancreatic beta cell mass of the NOD mice at the onset of diabetes. The p38 inhibitor has been shown to reduces p38 MAPK activity in these cells. Furthermore, the p38 inhibitor has been shown to prevent the development of diabetes and alleviates hyperglycemia in these mice by inhibiting T cell infiltration and preserving beta cell mass. It is hypothesized that HSP 60 is up-regulated in the NOD mice at onset (13 weeks) and before overt (18 weeks) diabetes and markedly suppressed in p38 inhibitor treated mice.

To examine one possible mechanism to explain the effectiveness of p38 inhibitors in preventing diabetes in NOD mice, female NOD mice were purchased from Jackson Labs, Bar Harbor, Me., USA and maintained in an animal facility. Mice were maintained in accordance with the guidelines of the Committee of Animals at Scios, Inc. All mice were kept under conventional conditions at a constant temperature (22-25° C.) and fed commercial powdered Purina chow diet and tap water ad libitum.

At 8 weeks the mice received an admixture of the p38 inhibitor in the powdered feed of the animals. Food consumption by the animals was approximately 3.5 g/day. The animals were divided into a vehicle group, which received no p38 inhibitor and the test group that received 600 mg/kg p38 inhibitor, equal to approximately a 1.8 μM circulating concentration of inhibitor. Beginning at 13 weeks, body weight, blood glucose levels were measured once a week before and after feeding, and the pancreata of the animals were subjected to immunohistochemical analysis to determine HSP 60 expression. At 18 weeks the final evaluations were performed.

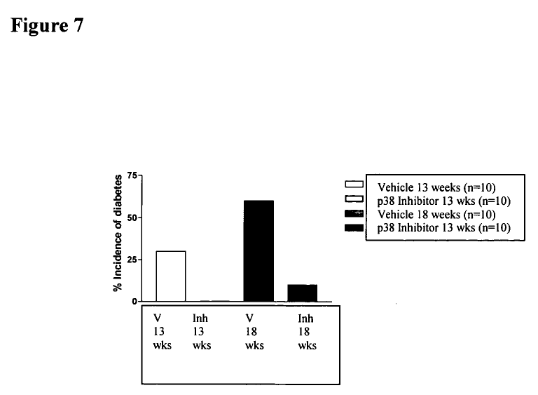

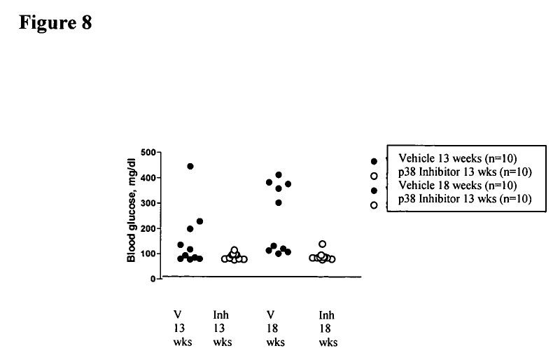

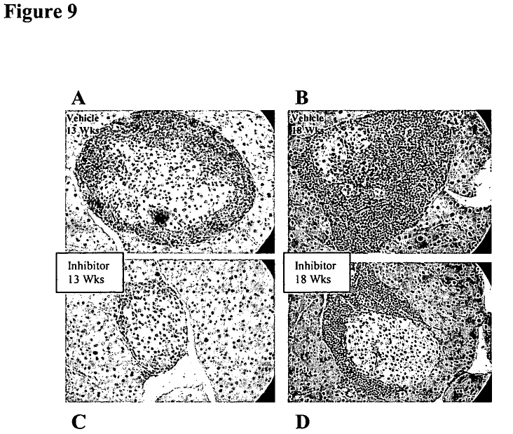

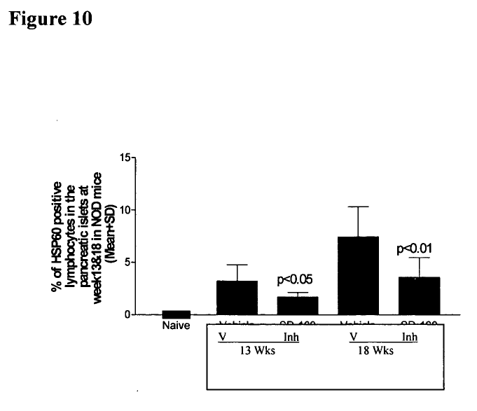

As shown in FIG. 7, administration of a p38 inhibitor prevented the development of Type-1 diabetes in NOD mice as measured at the onset (13 weeks) and before overt diabetes (18 weeks) time points. FIG. 8 shows that p38 inhibitor administration lowered blood glucose levels in NOD mice as compared to NOD that did not receive the p38 MAPK inhibitor. Immunohistochemical analysis of the pancreata of the animals in the study indicated that p38 inhibitor administration reduced HSP60 expression at the onset time point (13 weeks) and before the overt diabetes time point (18 weeks) in NOD mice (FIG. 9). Levels of HSP 60 expression in the NOD mice was examined and quantified as the percent of HSP 60 positive lymphocytes in pancreatic islets at week 13 and 18. As shown in FIG. 10, mice receiving the p38 MAPK inhibitor showed reduced levels expression of HSP 60 in pancreatic lymphocytes.

The results discussed above indicate that the administration of a p38 inhibitor prevents the development of diabetes and alleviates hyperglycemia in NOD mice by inhibiting T cell infiltration and preserving beta cell mass via p38 MAPK signaling pathway. The present study disclosed a positive correlation between the HSP 60 expression and the level of blood glucose levels at the onset (13 weeks) and before overt (18 weeks) of diabetes in NOD mice. These results suggest that the signal transduction pathway of the stress-induced expressions of HSP 60 in pancreatic beta cells may includes a process is sensitive to p38 MAP kinase. It is concluded that p38 pathway may acts as an enhancing factor in the activation of HSP 60 and p38 MAPK and HSP 60 involvement in regulatory loops of autoimmunity serve as basis for the development of strategies to prevent and/or treat autoimmune diseases even without knowledge of the causative (auto-) antigen.

All documents, e.g., scientific publications, patents and patent publications, recited herein are hereby incorporated by reference in their entirety to the same extent as if each individual document was specifically and individually indicated to be incorporated by reference in its entirety. Where the document cited only provides the first page of the document, the entire document is intended, including the remaining pages of the document.

1. A method of preventing Type I diabetes, comprising:
identifying a subject susceptible to Type I diabetes; and
administering to the subject a pharmaceutically effective amount of a p38 mitogen activated protein (MAP) kinase inhibitor sufficient to prevent onset of Type I diabetes.

2. The method of claim 1, wherein the subject presents an increased immune response against a heat shock protein.

3. The method of claim 2, wherein the heat shock protein is HSP 60.

4. The method of claim 1, wherein said p38 MAP kinase inhibitor is selective for p38α, p38β, p38γ, or p38δ.

5. The method of claim 1, wherein said patient is a human.

6. A method of decreasing blood glucose levels, comprising:
providing to a subject with elevated blood glucose levels with a pharmaceutically effective amount of a p38 mitogen activated protein (MAP) kinase inhibitor sufficient to decrease blood glucose level in said patient.

7. The method of claim 6, wherein said p38 MAP kinase inhibitor is selective for p38α, p38β, p38γ, or p38δ.

8. The method of claim 6, wherein said patient is a human.

9. A method of inhibiting body weight loss in a subject suffering from Type I diabetes, comprising:
identifying the subject suffering from to Type I diabetes; and
providing the subject with a pharmaceutically effective amount of a p38 mitogen activated protein (MAP) kinase inhibitor sufficient to inhibit body weight loss in the subject.

10. The method of claim 9, wherein said p38 MAP kinase inhibitor is selective for p38α, p38β, p38γ, or p38δ.

11. The method of claim 9, wherein said patient is a human.
